(19) United States
(12) Patent Application Publication
(10) Pub. No.: US 2020/0388362 A1
(43) Pub. Date: Dec. 10, 2020

(54) HEALTHCARE DATA CHIP DEVICE

(71) Applicant: HealthCare PayCard, LLC, Richardson, TX (US)

(72) Inventors: Jeffery Christian Blankinship, Plano, TX (US); Paul Edwards Clampitt, Plano, TX (US)

(21) Appl. No.: 16/942,653

(22) Filed: Jul. 29, 2020

Related U.S. Application Data
(63) Continuation-in-part of application No. 16/583,132, filed on Sep. 25, 2019.
(60) Provisional application No. 62/857,990, filed on Jun. 6, 2019.
(60) Provisional application No. 62/881,339, filed on Jul. 31, 2019.

(51) Int. Cl. G16H 10/65; G06Q 20/20; G16H 70/60

(52) U.S. Cl. CPC G16H 10/65; G16H 70/60; G06Q 20/208

(57) ABSTRACT

Techniques are described herein that are capable of accessing healthcare data using a healthcare data chip device. For instance, communication may be established with a point-of-sale reader on behalf of a user. The communication initiates a purchase of good(s) and/or service(s). The user may be authenticated to a store that stores aggregated healthcare data associated with the user by providing credentials of the user to the store. Access to the aggregated healthcare data, which includes portions that are aggregated from respective healthcare data storage systems, may be obtained via the point-of-sale reader, based at least in part on the credentials of the user and reference credentials being same. At least some of the aggregated healthcare data may be caused to be presented via a user interface that is included in the point-of-sale reader based at least in part on access to the aggregated healthcare data being obtained.

### CROSS-REFERENCE TO RELATED APPLICATION(S)

This application is a continuation-in-part of U.S. patent application Ser. No. 16/583,132 (Atty Docket No. H01.00010001), entitled “Restricted-Access Credit Device” and filed on Sep. 25, 2019, which claims the benefit of U.S. Provisional Patent Application No. 62/857,990 (Atty Docket No. H01.00010000), entitled “Restricted-Access Credit Card” and filed on Jun. 6, 2019, which are incorporated herein by reference in their entireties. This application also claims the benefit of U.S. Provisional Patent Application No. 62/881,339 (Atty Docket No. H01.00020000), entitled “Healthcare Data Chip Card” and filed on Jul. 31, 2019, which is incorporated herein by reference in its entirety.

### BACKGROUND

Over the past decade, much of the burden of paying for healthcare has shifted from employers to employees, and the costs for healthcare have increased substantially. For instance, such costs may relate to insurance deductibles, copayments (a.k.a. co-pays), medical procedures, medicines, preventative tests, and medical examinations. People may not have funds immediately available to cover these costs. Accordingly, the people may choose to delay or forego healthcare expenditures, which may negatively affect their health. For example, some people are unable to afford their insurance deductibles until many months (e.g., six months) into the year of coverage. In another example, some people wait for a major medical expense to take place before trying to get funding for such an expense.

The medical industry utilizes a variety of disparate software systems to store health records and medical records. For instance, each provider (or group of providers) of healthcare services may have its own software system. These software systems typically have their own interfaces for enabling access to the records stored therein. The interfaces of the various software systems typically are not compatible with the interfaces of the other software systems. Accordingly, each software system typically is not capable of communicating with the other software systems.

Health records and medical records traditionally are not consolidated, and physicians generally do not interact or collaborate with each other in the care of a patient unless the physicians work for the same employer (e.g., same hospital, same network, etc.). The result is that a patient's treatment for a condition may be disjointed. Each time the patient visits another physician, the physician typically starts from scratch, without knowledge of or access to information gathered or generated by previous physicians, to determine the condition and the proper treatment. The lack of consolidation of health records and medical records and the lack of oversight of such records may lead to mistreatment, overtreatment, undertreatment, over-prescription, lack of disclosure, lack of information, disparity among the health records and the medical records, which may contribute to relatively high healthcare costs.

### SUMMARY

Various approaches are described herein for, among other things, restricting access to a line of credit associated with a restricted-access credit device and/or accessing healthcare data using a healthcare data chip device. A restricted-access credit device is a credit device that controls whether purchase transactions are executed depending on whether merchant category codes of merchants associated with the purchase transactions are healthcare-related (e.g., healthcare-specific) merchant category codes. The terms “merchant category code” and “healthcare-related merchant category code” are discussed in further detail below. If the merchant category code of the merchant associated with a purchase transaction is a healthcare-related merchant category code, the restricted-access credit device may allow the purchase transaction to be executed, assuming that any other criteria are also satisfied. If such other criteria are not satisfied, the restricted-access credit device may not allow the purchase transaction to be executed (e.g., may prevent the purchase transaction from being executed). If the merchant category code of the merchant associated with a purchase transaction is not a healthcare-related merchant category code, the restricted-access credit device may not allow the purchase transaction to be executed. A credit device is a device with which a line of credit is associated to enable a user to whom the line of credit is granted to pay a merchant for goods and/or services by using the line of credit. The credit device is issued to the user by an issuer based at least in part on a promise by the user to pay the issuer for charges applied against the line of credit.

A merchant category code (MCC) is a number (e.g., four-digit number) identified in the ISO 18245 standard, which pertains to retail financial services. A merchant category code is assigned to a business to indicate the types of goods and/or services that the business provides (e.g., sells). For instance, a committee of issuers (e.g., MasterCard, Visa, American Express, Diners Club, and Discover) may create the merchant category codes to be used by merchants who use the payment infrastructure of any one or more of the issuers for purchase transactions. A healthcare-related merchant category code is a merchant category code that indicates that the types of goods and/or services that a business provides are healthcare-related goods and/or healthcare-related services. Examples of a healthcare-related merchant category code include but are not limited to the following: “4119” which pertains to ambulance services; “5047” which pertains to dental, lab, medical, and ophthalmic hospital equipment and supplies; “5122” which pertains to drugs, drug proprietaries, and druggists sundries; “5912” which pertains to drug stores and pharmacies; “5975” which pertains to hearing aids sales, service, and supply stores; “5976” which pertains to orthopedic goods and artificial limbs stores; “7277” which pertains to debt, marriage, and personal counseling service; “8011” which pertains to doctors not elsewhere classified; “8021” dentists and orthodontists; “8031” which pertains to osteopathic physicians; “8041” which pertains to chiropractors; “8042” which pertains to optometrists and ophthalmologists; “8043” which pertains to opticians, optical goods, and eyeglasses; “8049” which pertains to chiropodists and podiatrists; “8050” which pertains to nursing and personal care facilities; “8062” which pertains to hospitals; “8071” which pertains to dental and medical laboratories; “8099” which pertains to health practitioners and medical services not elsewhere classified; and “6300” which pertains to insurance sales, underwriting, and premiums. These example healthcare-related merchant category codes are provided for non-limiting illustrative purposes. For example, additional healthcare-related merchant category codes may be identified in the ISO 18245 standard in the future, and the embodiments described herein are intended to encompass such healthcare-related merchant category codes. In another example, a format of the merchant category codes (including the healthcare-related merchant category codes) may change in the future (e.g., to include one or more additional digits), and the embodiments described herein are intended to encompass such format changes.

A healthcare data chip device is a device that includes circuitry configured to enable a user to pay for good(s) and/or service(s) and further configured to provide access to healthcare data. The circuitry may include one or more chips (e.g., integrated circuit chip(s)) and/or one or more processors. Examples of healthcare data include but are not limited to health records (e.g., dental records), medical records, medical data, pharmaceutical data, dental data, provider data, and payment records for health service(s) and/or health-related good(s). The healthcare data may be associated with the user and/or member(s) of the user's family. For instance, the health records and medical records may specify doctors (e.g., family doctors, surgeons, dentists) visited by the user, dates of doctor visits, medical procedures performed on the user, etc. The medical data may specify conditions (e.g., diseases) that run in the user's family, conditions that the user has experienced (e.g., contracted), vital statistics of the user (e.g., over a period of time, such as the user's lifetime), and dental health. The pharmaceutical data may specify drugs that have been prescribed to the user, dates on which the drugs were prescribed, doctors who have prescribed the drugs to the user, and potential drug interactions. The provider data may include information that identifies providers of health services and/or health-related goods, services and goods offered by such providers, costs of such goods and services for each provider, information regarding doctors who provide health services for each provider thereof. The payment records may specify health services and health-related goods that have been purchased by the user, the dates on which the services and goods were purchased, and the costs of the services and goods.

The circuitry in the healthcare data chip device may store, access, transfer, modify, and/or restrict access to the healthcare data. For instance, the circuitry may limit access to the healthcare data to persons who are authorized to access the healthcare data. The healthcare data chip device may employ cryptographic techniques to secure the healthcare data and operations performed on the healthcare data. The healthcare data chip device may utilize any of a variety of technologies, including but not limited to EMV technology, nanotechnology, and blockchain technology to implement the functionality described herein.

EMV technology corresponds to a technical standard for smart payment devices (e.g., smart payment cards) and points-of-sale readers that are configured to interact with such smart payment devices. Such technical standards may be based on ISO/IEC 7816 (for contact devices) and ISO/IEC 14443 (for contactless devices). A contact device is a healthcare data chip device and/or a restricted-access credit device that is configured to interact with a point-of-sale reader by making physical contact with the point-of-sale reader. For instance, the physical contact may be between electrical terminal(s) in the healthcare data chip device or the restricted-access credit device and electrical and electrical terminal(s) in the point-of-sale reader. A contactless device is a healthcare data chip device and/or a restricted-access credit device that is capable of interacting with a point-of-sale reader without making physical contact with the point-of-sale reader. For instance, the contactless device may be configured to use near-field magnetic conduction communication or near-field communication to interact with the point-of-sale reader.

Nanotechnology includes manipulation of matter with at least one dimension sized from 1 to 100 nanometers (nm). Accordingly, nanotechnology may include manipulation on an atomic, molecular, and/or supramolecular scale. For instance, the functionalities described herein with regard to healthcare data chip devices and restricted-access credit devices may be implemented at a nanoscopic scale.

Blockchain technology generates a list of records (a.k.a. blocks) that are linked cryptographically. A record is added to the list based on each occurrence of a triggering event (e.g., a purchase transaction). Each record includes a cryptographic hash of the record that was most recently added to the list prior to the respective record that includes the cryptographic hash, a timestamp associated with the record (e.g., corresponding to and/or indicating a time at which the record is added to the list), and potentially other information. For example, the other information may include a Merkle tree. A Merkle tree is a tree in which each leaf node is identified (e.g., labelled) with a cryptographic hash of a corresponding record and each non-leaf node is identified with a cryptographic hash of the identifiers (e.g., labels) of its child nodes.

The healthcare data chip devices and restricted-access credit devices described herein may include any one or more of the above-referenced technologies to implement the functionalities described herein. In an example implementation, the healthcare data chip device includes an EMV chip for executing transactions for health services and health-related goods and a separate chip for providing access to the healthcare data. In another example implementation, the functionality of executing the transactions and the functionality for providing access to the healthcare data are implemented in a common (e.g., single) chip. References to EMV are provided for illustrative purposes and are not intended to be limiting. It will be recognized that any suitable transaction technology may be used in addition to or in lieu of EMV.

A healthcare data chip device and/or a restricted-access credit device may be in the form of a card (e.g., credit card and/or chip card (a.k.a. smart card)), a memory stick, a wearable device (e.g., ring, glasses, bracelet), or an implantable device. The healthcare data chip device and/or the restricted-access credit device may be implemented using software, firmware, hardware (e.g., electrical circuitry), or any combination thereof.

In a first example approach, a restricted-access credit device includes memory and processor(s) coupled to the memory. The processor(s) are configured to communicate with a point-of-sale terminal by providing an account identifier that identifies a line of credit associated with the restricted-access credit device to the point-of-sale terminal and further by receiving a purchase request that requests execution of a purchase transaction for an item from the point-of-sale terminal. The purchase request includes a merchant category code of a merchant that is associated with the point-of-sale terminal. The item includes good(s) and/or service(s). The processor(s) are further configured to cause the merchant category code of the merchant to be cross-referenced with healthcare-related merchant category codes. The processor(s) are further configured to selectively trigger execution of the purchase transaction depending on whether the merchant category code of the merchant corresponds to at least one of the healthcare-related merchant category codes.

In a second example approach, a healthcare data chip device includes memory and processor(s) coupled to the memory. The processor(s) are configured to establish communication with a point-of-sale reader on behalf of a user. The communication initiates a purchase of good(s) and/or service(s). The processor(s) are further configured to authenticate the user to a store that stores aggregated healthcare data associated with the user by providing credentials of the user to the store. The processor(s) are further configured to obtain access to the aggregated healthcare data via the point-of-sale reader based at least in part on the credentials of the user and reference credentials being same. The aggregated healthcare data includes portions that are aggregated from respective healthcare data storage systems that do not share the respective portions of the aggregated healthcare data with others of the healthcare data storage systems. The processor(s) are further configured to cause at least some of the aggregated healthcare data to be presented via a user interface that is included in the point-of-sale reader based at least in part on access to the aggregated healthcare data being obtained.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Moreover, it is noted that the invention is not limited to the specific embodiments described in the Detailed Description and/or other sections of this document. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate embodiments of the present invention and, together with the description, further serve to explain the principles involved and to enable a person skilled in the relevant art(s) to make and use the disclosed technologies.

FIG. 1 shows an example restricted-access credit system in accordance with an embodiment.

FIG. 2 depicts a flowchart of an example method for restricting access to a line of credit associated with a restricted-access credit device in accordance with an embodiment.

FIG. 3 depicts a flowchart of an example method for selectively adding additional credit to a line of credit associated with a restricted-access credit device in accordance with an embodiment.

FIGS. 4 and 7 are block diagrams of example restricted-access credit devices in accordance with embodiments.

FIG. 5 depicts a flowchart of an example method for selectively soliciting the user to join a credit union in accordance with an embodiment.

FIG. 6 depicts a flowchart of an example method for selectively notifying the user of credit union(s) in accordance with an embodiment.

FIG. 8 shows an example healthcare data chip system in accordance with an embodiment.

FIGS. 9-11 depict flowcharts of example methods for accessing healthcare data using a healthcare data chip device in accordance with embodiments.

FIG. 12 is a block diagram of an example healthcare data chip device in accordance with an embodiment.

FIG. 13 depicts an example implementation of a transaction executing chip shown in FIG. 12 in accordance with an embodiment.

FIG. 14 is a system diagram of an example mobile device in accordance with an embodiment.

FIG. 15 depicts an example computer in which embodiments may be implemented.

The features and advantages of the disclosed technologies will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

### I. Introduction

The following detailed description refers to the accompanying drawings that illustrate exemplary embodiments of the present invention. However, the scope of the present invention is not limited to these embodiments, but is instead defined by the appended claims. Thus, embodiments beyond those shown in the accompanying drawings, such as modified versions of the illustrated embodiments, may nevertheless be encompassed by the present invention.

References in the specification to “one embodiment,” “an embodiment,” “an example embodiment,” or the like, indicate that the embodiment described may include a particular feature, structure, or characteristic, but every embodiment may not necessarily include the particular feature, structure, or characteristic. Moreover, such phrases are not necessarily referring to the same embodiment. Furthermore, when a particular feature, structure, or characteristic is described in connection with an embodiment, it is submitted that it is within the knowledge of one skilled in the relevant art(s) to implement such feature, structure, or characteristic in connection with other embodiments whether or not explicitly described.

### II. Example Embodiments

Example embodiments described herein are capable of restricting access to a line of credit associated with a restricted-access credit device and/or accessing healthcare data using a healthcare data chip device. A restricted-access credit device is a credit device that controls whether purchase transactions are executed depending on whether merchant category codes of merchants associated with the purchase transactions are healthcare-related (e.g., healthcare-specific) merchant category codes. The terms “merchant category code” and “healthcare-related merchant category code” are discussed in further detail below. If the merchant category code of the merchant associated with a purchase transaction is a healthcare-related merchant category code, the restricted-access credit device may allow the purchase transaction to be executed, assuming that any other criteria are also satisfied. If such other criteria are not satisfied, the restricted-access credit device may not allow the purchase transaction to be executed (e.g., may prevent the purchase transaction from being executed). If the merchant category code of the merchant associated with a purchase transaction is not a healthcare-related merchant category code, the restricted-access credit device may not allow the purchase transaction to be executed. A credit device is a device with which a line of credit is associated to enable a user to whom the line of credit is granted to pay a merchant for goods and/or services by using the line of credit. The credit device is issued to the user by an issuer based at least in part on a promise by the user to pay the issuer for charges applied against the line of credit. The credit device may be a credit card and/or a smart card, though the scope of the example embodiments is not limited in this respect.

A merchant category code (MCC) is a number (e.g., four-digit number) identified in the ISO 18245 standard, which pertains to retail financial services. A merchant category code is assigned to a business to indicate the types of goods and/or services that the business provides (e.g., sells). For instance, a committee of issuers (e.g., MasterCard, Visa, American Express, Diners Club, and Discover) may create the merchant category codes to be used by merchants who use the payment infrastructure of any one or more of the issuers for purchase transactions. A healthcare-related merchant category code is a merchant category code that indicates that the types of goods and/or services that a business provides are healthcare-related goods and/or healthcare-related services. Examples of a healthcare-related merchant category code include but are not limited to the following: “4119” which pertains to ambulance services; “5047” which pertains to dental, lab, medical, and ophthalmic hospital equipment and supplies; “5122” which pertains to drugs, drug proprietaries, and druggists sundries; “5912” which pertains to drug stores and pharmacies; “5975” which pertains to hearing aids sales, service, and supply stores; “5976” which pertains to orthopedic goods and artificial limbs stores; “7277” which pertains to debt, marriage, and personal counseling service; “8011” which pertains to doctors not elsewhere classified; “8021” dentists and orthodontists; “8031” which pertains to osteopathic physicians; “8041” which pertains to chiropractors; “8042” which pertains to optometrists and ophthalmologists; “8043” which pertains to opticians, optical goods, and eyeglasses; “8049” which pertains to chiropodists and podiatrists; “8050” which pertains to nursing and personal care facilities; “8062” which pertains to hospitals; “8071” which pertains to dental and medical laboratories; “8099” which pertains to health practitioners and medical services not elsewhere classified; and “6300” which pertains to insurance sales, underwriting, and premiums. These example healthcare-related merchant category codes are provided for non-limiting illustrative purposes. For example, additional healthcare-related merchant category codes may be identified in the ISO 18245 standard in the future, and the embodiments described herein are intended to encompass such healthcare-related merchant category codes. In another example, a format of the merchant category codes (including the healthcare-related merchant category codes) may change in the future (e.g., to include one or more additional digits), and the embodiments described herein are intended to encompass such format changes.

In an example implementation, MCCs may be used to determine the type of services a health provider is allowed to perform or may be used to provide a list of allowed health service options to the user. For example, the user may be provided with a list of pre-approved prescription drugs from specified merchants for a condition. The list may exclude drugs that are not pre-approved. In another example implementation, the MCCs may be used to define rules and restrictions for specified types of procedures or doctors of specified specialty or geographic location. In another example implementation, information regarding healthcare-related purchases, including the corresponding MCCs, may be automatically reported to the Internal Revenue Services for tax purposes.

A healthcare data chip device is a device that includes circuitry configured to enable a user to pay for good(s) and/or service(s) and further configured to provide access to healthcare data. The circuitry may include one or more chips (e.g., integrated circuit chip(s)) and/or one or more processors. Examples of healthcare data include but are not limited to health records (e.g., dental records), medical records, medical data, pharmaceutical data, dental data, provider data, and payment records for health service(s) and/or health-related good(s). The healthcare data may be associated with the user and/or member(s) of the user's family. For instance, the health records and medical records may specify doctors (e.g., family doctors, surgeons, dentists) visited by the user, dates of doctor visits, medical procedures performed on the user, etc. The medical data may specify conditions (e.g., diseases) that run in the user's family, conditions that the user has experienced (e.g., contracted), vital statistics of the user (e.g., over a period of time, such as the user's lifetime), and dental health. The pharmaceutical data may specify drugs that have been prescribed to the user, dates on which the drugs were prescribed, doctors who have prescribed the drugs to the user, and potential drug interactions. The provider data may include information that identifies providers of health services and/or health-related goods, services and goods offered by such providers, costs of such goods and services for each provider, information regarding doctors who provide health services for each provider thereof. The payment records may specify health services and health-related goods that have been purchased by the user, the dates on which the services and goods were purchased, and the costs of the services and goods.

The circuitry in the healthcare data chip device may store, access, transfer, modify, and/or restrict access to the healthcare data. For instance, the circuitry may limit access to the healthcare data to persons who are authorized to access the healthcare data. The healthcare data chip device may employ cryptographic techniques to secure the healthcare data and operations performed on the healthcare data. The healthcare data chip device may utilize any of a variety of technologies, including but not limited to EMV technology, nanotechnology, and blockchain technology to implement the functionality described herein.

EMV technology corresponds to a technical standard for smart payment devices (e.g., smart payment cards) and points-of-sale readers that are configured to interact with such smart payment devices. Such technical standards may be based on ISO/IEC 7816 (for contact devices) and ISO/IEC 14443 (for contactless devices). A contact device is a healthcare data chip device and/or a restricted-access credit device that is configured to interact with a point-of-sale reader by making physical contact with the point-of-sale reader. For instance, the physical contact may be between electrical terminal(s) in the healthcare data chip device or the restricted-access credit device and electrical and electrical terminal(s) in the point-of-sale reader. A contactless device is a healthcare data chip device and/or a restricted-access credit device that is capable of interacting with a point-of-sale reader without making physical contact with the point-of-sale reader. For instance, the contactless device may be configured to use near-field magnetic conduction communication or near-field communication to interact with the point-of-sale reader.

The healthcare data chip device may include an EMV chip that is capable of extracting and delivering healthcare data. It should be noted that EMV chips traditionally have been available solely for providing payments. The healthcare data chip device may include another chip that is configured to extract and deliver healthcare data. It will be recognized that the EMV chip and the other chip may be combined into a common chip. The healthcare data chip device may be configured to adhere to the Payment Card Industry (PCI) data security standard and Health Insurance Portability and Accountability Act (HIPAA) requirements.

Nanotechnology includes manipulation of matter with at least one dimension sized from 1 to 100 nanometers (nm). Accordingly, nanotechnology may include manipulation on an atomic, molecular, and/or supramolecular scale. For instance, the functionalities described herein with regard to healthcare data chip devices and restricted-access credit devices may be implemented at a nanoscopic scale.

Blockchain technology generates a series of records (a.k.a. blocks) that are linked cryptographically. The series of blocks is referred to as a “blockchain”. A record is added to the series based on each occurrence of a triggering event (e.g., a purchase transaction). Each block includes a link to the block (e.g., cryptographic hash of the block) that was most recently added to the series prior to the respective block that includes the link, a timestamp associated with the block (e.g., corresponding to and/or indicating a time at which the block is added to the series), and potentially other information. For example, the other information may include a Merkle tree. A Merkle tree is a tree in which each leaf node is identified (e.g., labelled) with a cryptographic hash of a corresponding block and each non-leaf node is identified with a cryptographic hash of the identifiers (e.g., labels) of its child nodes. The blocks can be updated, but the blocks cannot be erased. Inviolability and historicity of data are two example features of data at the functional level, “the data level”. With regard to inviolability and historicity of data, blockchain may ensure that blocks are tracked in their correct chronological order, which may prevent a posteriori reconstruction of blocks. Data integrity may be ensured by the cryptographic validation of each transaction. Cryptographic validation of each transaction may facilitate (e.g., ensure) sincerity of authentication. Traceability and historicity of the data are functionalities of the technology. Each transaction is timestamped. The user owns a copy of proof of the time-stamp associated with each block. Thus, the existence of data in each block becomes provable while the data remain confidential.

The healthcare data chip device may use blockchain technology to retrieve (e.g., compile), store, and/or process healthcare data, including transactional information to facilitate maintenance of transaction histories. Blockchain may serve as a decentralized public ledger (e.g., data store) in which the records (e.g., proof of existence of data, such as healthcare data, which may include transactional information) are recorded in a secure and verifiable way. For instance, the healthcare data chip device may store identifiers associated with respective transactions. The ledger is owned and controlled by users and is not ruled by a trusted third party or central regulatory entity. Trust is encoded into the protocol and maintained by the community of users. Accordingly, blockchain may eliminate a need for a third party to process transactions that are performed with the healthcare data chip device. Continuous and consistent medical care, data sharing, and personal data privacy are some of the challenges that are faced by the healthcare industry. Blockchain technology may be employed to address these challenges.

The healthcare data chip devices and restricted-access credit devices described herein may include any one or more of the above-referenced technologies to implement the functionalities described herein. In an example implementation, the healthcare data chip device includes an EMV chip for executing transactions for health services and health-related goods and a separate chip for providing access to the healthcare data. In another example implementation, the functionality of executing the transactions and the functionality for providing access to the healthcare data may be implemented in a common (e.g., single) chip. References to EMV are provided for illustrative purposes and are not intended to be limiting. It will be recognized that any suitable transaction technology may be used in addition to or in lieu of EMV.

A healthcare data chip device and/or a restricted-access credit device may be in the form of a card (e.g., credit card and/or chip card (a.k.a. smart card)), though the scope of the example embodiments is not limited in this respect. For instance, the healthcare data chip device and/or the restricted-access credit device may be in the form of a memory stick, a wearable device (e.g., ring, glasses, bracelet), or an implantable device. The healthcare data chip device and/or the restricted-access credit device may be implemented using software, firmware, hardware (e.g., electrical circuitry), or any combination thereof.

Healthcare data chip devices and restricted-access credit devices may be issued by banks or credit unions. In some example embodiments, one or more credit unions may be members of a credit union service organization (CUSO). A CUSO is a corporate entity that is owned at least in part by one or more federally chartered or federally insured, state-chartered credit unions. For instance, the credit union(s) may own at least 1% of the CUSO. Unlike a bank, a CUSO is regulated by the National Credit Union Administration (NCUA) and may be affiliated with the National Association of Credit Union Service Organizations (NACUSO), which is a membership-based advocacy organization that provides members a shared collaborative platform to work together on innovation. A CUSO may be organized as a corporation, a limited liability corporation, or a limited partnership. If the CUSO is organized as a limited partnership, a credit union that is a member of the CUSO can participate as a limited partner and not as a general partner.

If a credit union has expertise in providing quality services in one service type area, it can form a CUSO to provide these services to other credit unions and potentially non-credit union members. For example, a credit union may have expertise in providing call center services to other credit unions (or non-credit union members). Investments of CUSOs in non-CUSO service providers and general activities of the CUSOs are regulated by NCUA. Title 12, Chapter VII, Subchapter A, Part 712, Section 712.5 of the Code of Federal Regulations delineates the types of activities and services that are pre-approved for CUSOs. The NCUA may at any time, based on supervisory, legal, or safety and soundness reasons, limit any CUSO activities or services or refuse to permit any CUSO activities or services. The Code limits CUSOs to certain activities and services. Examples of such activities and services include but are not limited to checking and currency services; clerical, professional and management services; business loan origination; consumer mortgage loan origination; electronic transaction services; financial counseling services; fixed asset services; insurance brokerage or agency; leasing; loan support services; record retention, security and disaster recovery services; securities brokerage services; shared credit union branch (service center) operations; student loan origination; travel agency services; trust and trust-related services; real estate brokerage services; CUSO investments in non-CUSO service providers; credit card loan origination; and payroll processing services. CUSOs are not allowed to acquire control of another depository financial institution; invest in shares, stock, or obligations of an insurance company, trade association, liquidity facility, or similar organization, corporation, or association. A CUSO may have other limitations, such as charging no more than an 18% annual percentage rate (APR) on an outstanding balance on a line of credit.

A CUSO may be formed for any of a variety of reasons. Membership of a credit union in a CUSO may provide avenues for innovation, creativity, and revenue streams that would not be available within the confines of the credit union. Membership in the CUSO may reduce service costs incurred by the credit union. Membership in the CUSO may enable the credit union to provide faster, more comprehensive, and cheaper service and new services that credit union alone may not be able to provide. For example, not all credit unions have the capital to gain expertise in originating business and commercial real estate loans. If several credit unions pool their resources, they can afford to hire the right individuals and in turn provide a valuable service to their members.

In CUSO embodiments, the field of membership of each credit union that is a member of the CUSO may be eligible to utilize a sponsored device. The sponsored device is a sponsored healthcare data chip device and/or a sponsored restricted-access credit device. Up to 49% of the users who receive a sponsored device may have no membership in any of the credit union(s) that are members of the CUSO. The CUSO may provide any of a variety of services, including but not limited to

A field of membership (FOM) of a credit union is defined by pre-determined criteria for obtaining membership in the credit union. The pre-determined criteria may be set forth in the credit union's charter. The Federal Credit Union Act recognizes three types of federal credit union charters: (1) single common bond (occupational and associational), (2) multiple common bond (more than one group with each group having a common bond of occupation or association), and (3) community. An occupational common-bond is an employer-based group of persons employed within a trade, industry, or profession. An associational common bond is a member-based group meeting the NCUA's threshold requirement and totality of circumstances test. A community is a geographical area (e.g., neighborhood) meeting the NCUA's definition of a well-defined local community or rural district. The type of charter under which a credit union operates determines what groups or geographic areas it may serve. Some attributes of person that may be taken into consideration to determine whether the person is eligible for membership in the credit union include but are not limited to place of residency (e.g., city or state), location of employer, identity of employer, location of place of worship, religious affiliation, sexual orientation, employer, and military service (including branch of military). It will be recognized that the FOM of the credit union encompasses (a) members of the credit union and (b) persons who satisfy the pre-determined criteria and who have not become members of the credit union.

Example techniques described herein have a variety of benefits as compared to conventional techniques for handling processing of healthcare expenditures and/or accessing healthcare data. For instance, the example techniques may enable a user to obtain healthcare, by providing a line of credit (e.g., via an employer-sponsored restricted access credit device) from which the related healthcare expenses may be paid, that the user would not have otherwise been able to obtain or that the user would have been required to delay due to a lack of funds. Accordingly, the example techniques may increase health and/or quality of life of the user. The example techniques may facilitate tracking and organization of healthcare-related purchase transactions. For example, the example techniques may provide a restricted-access credit device that is configured to handle requests for all such transactions and that is further configured to not handle (e.g., to deny) requests for non-healthcare-related purchase transactions.

In another example, the example techniques may provide a healthcare data chip device that is configured to distribute (e.g., securely upload using encryption and/or authentication) healthcare data, such as electronic health records (EHRs) and electronic medical records (EMRs), associated with the user among multiple incompatible healthcare data storage systems and/or download (e.g., securely download using encryption and/or authentication) healthcare data from the various healthcare data storage systems (e.g., from any provider, health system, or software system). The healthcare data chip device may upload the healthcare data via a proprietary website (e.g., mydoctorsnote.com) to facilitate storage, transfer, discharge, and downloading of the healthcare data. A mobile application may be used as an intermediate access point to facilitate data transfer between the healthcare data chip device, the cloud (e.g., a cloud-based server), the consumer, the physician, and the provider. For example, a healthcare provider may be able to access a user's healthcare information by logging into a URL, such as mydoctorsnotes.com, using the requisite credentials (e.g., username, password, and/or PIN). In another example, the user may store the user's information via the URL, and doctors may log into the URL to access the user's healthcare data.

Accordingly, the healthcare data chip device may enable a user to access and combine the user's own EHRs and EMRs. An EHR is electronic health information of an individual patient and/or an individual population. An EMR is health information of a patient that is created by provider(s) to document encounters of the patient in hospitals and ambulatory environments. An EMR may serve as a data source for an EHR. The healthcare data chip device may be capable of aggregating the EHRs and the EMRs from the healthcare data storage systems regardless whether the healthcare data storage systems are incompatible with each other. For instance, the healthcare data chip device may be capable of providing an interface among the healthcare data storage systems so that cross-communication and data sharing is possible to reduce disparate EHRs and EMRs. The healthcare data chip device may enable consolidation of a user's healthcare data to one source or to a limited number of sources. For example, EMR software systems, such as Allscripts, NextGen, Meditech, Cerner, and Epic, are being used by many different hospitals and physicians' offices. These EMRs may be integrated in the healthcare data chip device and/or in a store that is accessible to the healthcare data chip device to provide a common source from which the data may be accessible. The healthcare data chip device may be capable of sharing the healthcare data that are received from each healthcare data storage system with each of the other healthcare data storage systems. The healthcare data chip device may enable users (e.g., patients) to keep their healthcare data in their possession, to control access to their healthcare data, and to share their healthcare data with their healthcare specialists, including but not limited to physicians, nurses, technicians, pharmacists, and insurance companies.

Accordingly, the example techniques may increase efficiency of gathering a user's healthcare data, including information regarding healthcare-related purchase transactions of the user, and/or generating documentation (e.g., tax statement, prescription history, medical procedure history, medical screening or testing history) based on such information.

Using a restricted-access credit device and/or a healthcare data chip device described herein to pay for healthcare-related purchases may increase a rate at which insurance claims related to the healthcare-related purchases are processed. Accordingly, using the restricted-access credit device and/or the healthcare data chip device may reduce an amount of time that is required for the insurance claims to be processed. For instance, the restricted-access credit device and/or the healthcare data chip device may cause information regarding a purchase transaction to be automatically uploaded to a server, which may trigger the server to automatically process an insurance claim associated with the purchase transaction. If a portion (e.g., 50% or all) of the cost of an item that is purchased via the purchase transaction is covered by insurance, the server may reimburse the portion of the cost to a line of credit associated with the restricted-access credit device and/or the healthcare data chip device before a payment related to the purchase transact is due. Accordingly, the user may receive the item without an out-of-pocket expense. The example techniques described herein may provide real-time (e.g., instant) analysis of the user's credit needs, medical needs, etc. to determine whether additional credit is to be added to the line of credit.

Using a healthcare data chip device described herein to consolidate portions of healthcare data of a user from multiple healthcare data storage system (e.g., that are incompatible and/or unable to communicate with each other) into aggregated healthcare data may increase efficiency of a user and/or a computer system that is used to access the portions of the healthcare data. Consolidation of the healthcare data may reduce a number of instances of mistreatment, overtreatment, undertreatment, over-prescription, lack of disclosure, lack of information, and/or disparity among health records and medical records. Accordingly, such consolidation may contribute to a reduction in healthcare costs. The healthcare data chip may be capable of providing access to the consolidated healthcare data via a point-of-sale device. Having the consolidated healthcare data available to a user at the point-of-sale may increase the efficiency of the user with regard to purchase transactions (e.g., by informing the user about discounts that are associated with healthcare-related goods and/or healthcare-related services that the user is considering for purchase, compatibility of such goods (e.g., drugs) with other goods, side effects associated with such goods, price limits established for the goods and/or services by a government institution such as Medicare or Medicaid, and other services that are available to the user).

Examples of such other services include but are not limited to insurance (e.g., life insurance, disability insurance, involuntary unemployment insurance) and debt cancellation. For instance, the user may take a DNA test to determine conditions (e.g., diseases) to which the user is predisposed. The user may be offered insurance coverage that covers at least one of the conditions to which the user is predisposed, meaning that the insurance coverage would entitle the user to designated payment(s) in the event that the user has the condition(s). Debt cancellation (a.k.a. debt relief) (a) partially or entirely forgives a debt of a user and/or (b) slows down or stops growth of the debt based on satisfaction of one or more criteria. For instance, debt cancellation may be configured to make minimum payment on a line of credit that is associated with the healthcare data chip device for a specified period of time (e.g., if the user becomes unable to work).

The healthcare data chip device may provide transparency in the healthcare services industry by placing control over users' healthcare data in the users' hands. The healthcare data may be protected by configuring the healthcare data chip device to utilize security measures such as fingerprint analysis, facial recognition, iris scans, retinal scans, body recognition (e.g., blood type, cellular structure). The healthcare data chip device may be implemented as a wearable device or an implantable device. The healthcare data chip device may contain pricing information regarding medical procedures and/or reimbursement information to enhance transparency and to enable the user to shop around and make informed medical decisions. For instance, the pricing information and the reimbursement information may include Medicare pricing and reimbursement information.

With the user's pharmaceutical data in one accessible place, cross-reaction of drugs may be prevented. The healthcare data chip device may be configured to execute a drug interaction checker to prevent harmful drug interactions. For example, mixing more than one medication or mixing a medication with certain foods, beverages, or over-the-counter medicines may increase a likelihood of a drug interaction. Not all drug interactions are serious, but those that are serious can be life threatening. The drug interaction checker may help the user understand the possible outcome of taking a prescribed medication before the user takes the medication. With the healthcare data chip device, the user may have the information readily available to check for drug interactions. The pharmacological database will also assist the physician in determining suitable drugs to prevent drug interactions and to prevent over-prescribing drugs.

The healthcare data chip device may control fraudulent obtainment of prescription drugs, healthcare services, and/or insurance claims. For instance, multiple (e.g., different) medical record numbers may exist for a single individual. In another example, multiple individuals may have the same medical record number. The healthcare data chip device may help to segregate, identify, and secure medical records for the user. It is common for one person to have multiple medical records. Every time an individual comes for treatment of a different condition, a new medical record may be established for that person, segmenting the individual's medical history. For example, a municipality may provide benefits to a resident whose marital status, employment, or address has changed. If the municipality does not conduct a census every year, the municipality may continue to provide medical benefits or insurance to that person who is no longer married to the insured. For example, in 2015, Dallas county had more than 60,000 people receiving benefits that were not even alive, employed, or a part of the insurance network to have those benefits. These inconsistencies in healthcare are reflected in medical records.

The healthcare data chip device may expedite and/or automate claims adjudication, transactions, or process patient checkouts of services rendered. “Claims adjudication” is a phrase used in the insurance industry to refer to the process of paying submitted claims or denying those claims after comparing the claims to the benefit or coverage requirements. The adjudication process includes receiving a claim from an insured person and then processing the claim to determine whether the claim is to be paid. For instance, a determination may be made whether a treatment that the user received and/or an amount that was charged for the treatment is allowed under the user's insurance policy. If the treatment and/or the amount are not allowed under the policy, the entire claim may be rejected, or the disallowed portion of the claim may be rejected and the allowed portion may be paid. The claim may be processed manually or by executing software that is configured to process the claim. If the claim is processed automatically using software (e.g., a web-based subscription service), the claim process is called “auto-adjudication.” Although automating claims often improves efficiency of the adjudication process and reduces expenses associated with manual claims adjudication, many claims are submitted on paper and are processed manually by insurance workers.

For example, a patient on Medicare who has a restricted-access credit device that is linked to a healthcare data chip device may use the restricted-access credit device to pay for the portion of the cost for health services and/or health-related goods that is not covered by, e.g., Part B Medicare or that is an out-of-pocket amount that is still due. By swiping or inserting the healthcare data chip device at a point-of-sale reader, one may access the published Medicare charge chart regarding fees for service and identify whether the patient was billed appropriately and accurately for the specified procedural code and/or service. The healthcare data chip device may utilize automated artificial intelligence (A.I.). technology. A doctor's transcription may be loaded onto the healthcare data chip device and/or uploaded to the cloud via the healthcare data chip device. The transcription may have the diagnosis code, the current procedural terminology (CPT) code to identify the procedures, etc. For instance, the transcription may be automatically generated during a procedure and automatically loaded onto the patient's healthcare data chip device. The patient's healthcare data chip device and/or the restricted-access credit device may be automatically billed. If the transaction involves Medicare, the patient's demographic information may be identified, and the Medicare reimbursement rate may be accurately determined for that particular procedure in that area of practice. This may reduce overbilling.

Insurance companies receive billions of claims to process electronically and/or manually, and they determine legitimacy of each claim. This process is relatively slow, often causing the reimbursement process to extend 30 to 60 days. In the late 1990s, cartels exploited the weaknesses of the claim adjudication process for Medicare claims to defraud the Medicare system of billions of dollars. The healthcare data chip device may expedite the claim adjudication and reimbursement processes, for example, by providing immediately available procedure codes and patient authentication. A.I. technology in the healthcare data chip device can apply the standards and rules of the Medicare program to catch non-allowed (e.g., fraudulent) billings.

Insurance companies are notorious for not paying claims due to minor clerical or typographical errors. For example, if letters in the insured user's name are transposed (e.g., “Jeff” is inadvertently spelled “eJff”) or the gender of the user is inadvertently listed incorrectly on the report, such errors often provide a sufficient reason for the insurance company to deny the claim. The longer insurance companies can withhold payment for claims, the more interest the insurance companies are making off of the money. Accordingly, the healthcare data chip device may implement processes (e.g., automatic generation of insurance claims for medical procedures) that increase accuracy of clinical documentation to reduce the amount of time and money expended to have claims paid. Automation and A.I.-based processes may be utilized to remove human error from the claim adjudication and reimbursement processes. Auto adjudication may eliminate the need for a human set of eyes to review claims (and to access another source for verification of the information therein) and to enter data regarding the claims.

The healthcare data chip device may include the identification of the user, the authentication of the user (e.g., down to the DNA level), the user's medical records, dental records, prescription records, financial information, payment history, and so forth. Rather than authenticating the user merely by matching the user's name, birthday, picture, and/or password, the healthcare data chip device can authenticate the user using the user's DNA profile, blood, saliva, fingerprint, iris scan, and/or retinal scans, to provide an unequivocal match. The healthcare data chip device can perform an unambiguous identification process that negates the need for picture identification, which can easily be duplicated.

The healthcare data chip device may save lives in the “golden fifteen minutes of life.” A person has approximately fifteen minutes from the time the person gets into a critical life or death situation to either live or die. For example, if the person gets into a car wreck and an ambulance arrives, the paramedic may see the person bleeding out and know nothing about that person. The paramedic may start a blood infusion, but the blood may be of the wrong blood type; or unbeknownst to the paramedic, the person might be allergic to certain drugs or treatments. If the person were to have a healthcare data chip device that identifies the person's basic vitals, such as gender, blood type, allergies, medicines, and diseases that the person is managing, any healthcare personnel can step into the situation and use the information from the healthcare data chip device to render appropriate care.

The healthcare data chip device may enable tracking of a location of the user. For instance, the healthcare data that is accessed by the healthcare data chip device may indicate a location at which each purchase transaction is conducted using the healthcare data chip device. A location of the user may be estimated by analyzing the locations of the purchase transactions and the times or dates on which the purchase transactions were conducted. For instance, the location of the user may be estimated to be at a location of the most recent purchase transaction that was conducted using the healthcare data chip device.

A restricted-access credit device and a healthcare data chip device may be combined into a unitary device. Accordingly, embodiments described herein with reference to an example restricted-access credit device are applicable to the example healthcare data chip devices described herein. Embodiments described herein with reference to an example healthcare data chip device are applicable to the example restricted-access credit devices described herein.

A proprietary cryptocurrency may be developed for use with restricted-access credit devices and healthcare data chip devices. For instance, the cryptocurrency may be used to provide rewards to users of the restricted-access credit devices and the healthcare data chip devices in return for the users using the restricted-access credit devices and the healthcare data chip devices for their health-related purchases. It should be noted that any suitable cryptocurrency may be used with restricted-access credit devices and healthcare data chip devices. Accordingly, the cryptocurrency need not necessarily be proprietary.

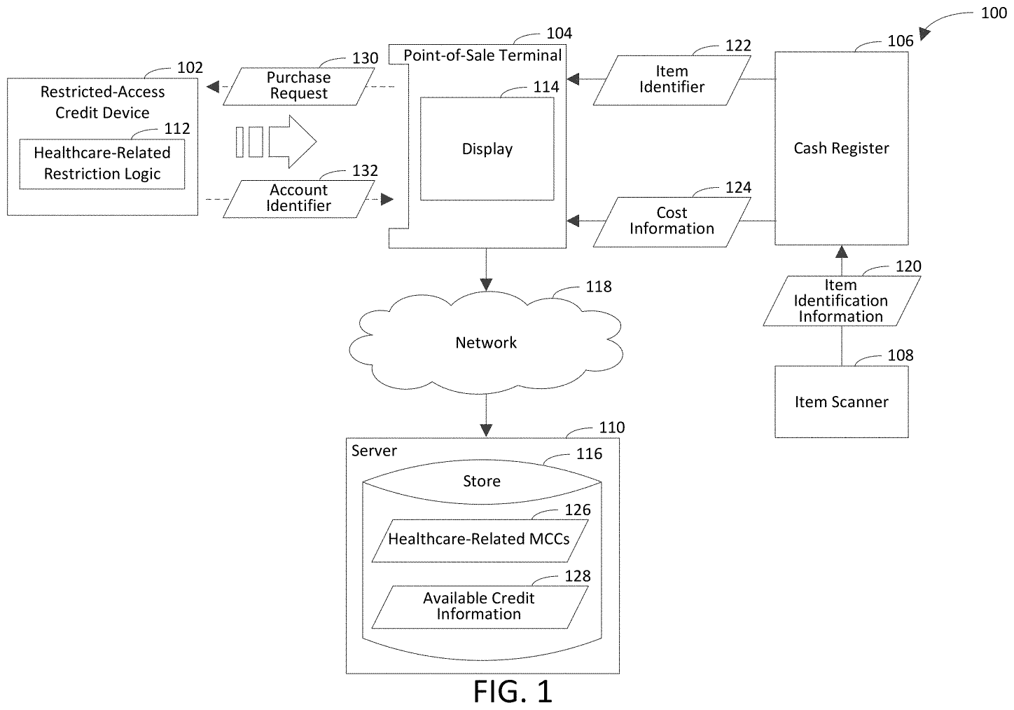

FIG. 1 shows an example restricted-access credit system 100 in accordance with an embodiment. Generally speaking, the restricted-access credit system 100 operates to restrict access to a line of credit associated with a restricted-access credit device 102. As shown in FIG. 1, the restricted-access credit system 100 includes the restricted-access credit device 102, a point-of-sale (POS) terminal 104 (a.k.a. POS reader 104), a cash register 106, an item scanner 108, a server 110, and a network 118. Communication among the POS terminal 104, the cash register 106, the item scanner 108, and the server 110 may be carried out over the network 118 using well-known network communication protocols. The network 118 may be a wide-area network (e.g., the Internet), a local area network (LAN), another type of network, or a combination thereof. The POS terminal 104 is shown in FIG. 1 to be coupled to the server 110 via the network 118 for non-limiting, illustrative purposes. The cash register 106 is shown to be coupled to the POS terminal 104 and the item scanner 108 without going through the network 118 for non-limiting, illustrative purposes to show that communication therebetween need not necessarily pass through the network 118. Communication between the restricted-access credit device 102 and the POS terminal 104 may be carried out over the network 118 and/or using any other suitable technology (e.g., near-field communication or physical contact). Any of the connections between the POS terminal 104, the cash register 106, the item scanner 108, and the server 110 may be wireless or wired.

The restricted-access credit device 102 is a processing system that is capable of communicating with the POS terminal 104. An example of a processing system is a system that includes at least one processor that is capable of manipulating data in accordance with a set of instructions. For instance, a processing system may be a chip card (e.g., an EMV chip card), a computer, a personal digital assistant, a wearable computer such as a smart watch or a head-mounted computer, a cellular telephone, etc. The processing system may be incorporated into any suitable object, including but not limited to a ring, a necklace, an implantable device, etc. For instance, the implantable device may be implanted beneath the skin of the user to whom the line of credit is granted or a pet of the user. It will be recognized that the user may be a human or a non-human animal (e.g., a pet). For example, the restricted-access credit device 102 may be used in a veterinary setting.

The restricted-access credit device 102 may be configured to communicate with the POS terminal 104 in accordance with the UnifiedPOS (UPOS) standard, which is managed by the Association for Retail Technology Standards (ARTS). A variety of platform-specific implementations of the UnifiedPOS standard exist. One example platform-specific implementation is Object Linking & Embedding (OLE) for Retail POS (OPOS), which was developed for Microsoft Windows® operating systems. The restricted-access credit device 102 and the POS terminal 104 may communicate in accordance with any such implementation of the UPOS standard.

Communication between the restricted-access credit device 102 and the POS terminal 104 may be initiated in any of a variety of ways. For example, the restricted-access credit device 102 may be physically inserted at least partially into the POS terminal 104. In accordance with this example, circuitry in the healthcare-related restriction logic 112 may come into physical contact with conductive leads in the POS terminal 104, thereby establishing a communication channel. For instance, the physical contact may form an electrical contact between the restricted-access credit device 102 and the POS terminal 104. In another example, the restricted-access credit device 102 may be placed within a designated proximity from the POS terminal 104. In accordance with this example, any of a variety of near-field communication protocols may be used to establish a communication channel between the restricted-access credit device 102 and the POS terminal 104. For instance, the communication channel may be established via electrical coupling and/or magnetic coupling.

The restricted-access credit device 102 includes healthcare-related restriction logic 112. The healthcare-related restriction logic 112 is configured to restrict access to the line of credit associated with the restricted-access credit device 102 in accordance with any one or more of the techniques described herein. In an example implementation, the healthcare-related restriction logic 112 communicates with the POS terminal 104 by receiving a purchase request 130 that requests execution of a purchase transaction for an item from the POS terminal 104. The purchase request 130 includes a merchant category code (MCC) of a merchant that is associated with the POS terminal 104. The item includes good(s) and/or service(s). In accordance with this implementation, the healthcare-related restriction logic 112 communicates with the POS terminal 104 further by providing an account identifier 132 that identifies the line of credit associated with the restricted-access credit device 102 to the POS terminal 104.

In one aspect, the account identifier 132 may include a transaction-agnostic account number that identifies the line of credit. A transaction-agnostic account number is an account number that is used for multiple (e.g., all) purchase transactions that are executed using a credit device (e.g., restricted-access credit device 102). Accordingly, in this aspect, the same transaction-agnostic account number may be associated with each purchase transaction that is executed using the restricted-access credit device 102.

In another aspect, the account identifier 132 may include a transaction-specific identifier (e.g., in lieu of the transaction-agnostic account number) that identifies the line of credit. A transaction-specific identifier is an identifier that is used for a single purchase transaction. For instance, a first transaction-specific identifier may identify the line of credit for a first purchase transaction; a second transaction-specific identifier that is different from the first transaction-specific identifier may identify the line of credit for a second purchase transaction; a third transaction-specific identifier that is different from the first and second transaction-specific identifiers may identify the line of credit for a third purchase transaction, and so on. Accordingly, the transaction-specific identifier that is associated with each purchase transaction may uniquely identify that purchase transaction. It will be recognized that a transaction-specific identifier provides greater security than a transaction-agnostic account number.

In further accordance with this implementation, the healthcare-related restriction logic 112 causes the MCC of the merchant to be cross-referenced with healthcare-related MCCs (e.g., healthcare-related MCCs 126). For example, the healthcare-related restriction logic 112 may cross-reference the MCC of the merchant with the healthcare-related MCCs 126. In accordance with this example, the healthcare-related restriction logic 112 may retrieve the healthcare-related MCCs 126 from the server 110 (e.g., via the POS terminal 104) for comparison with the MCC of the merchant. In another example, the healthcare-related restriction logic 112 may instruct the server 110 to cross-reference the MCC of the merchant with the healthcare-related MCCs 126. In accordance with this example, the healthcare-related restriction logic 112 may forward the MCC of the merchant to the server 110 (e.g., via the POS terminal 104) along with an instruction, which instructs the server 110 to cross-reference the MCC of the merchant with the healthcare-related MCCs 126. In further accordance with this example, the healthcare-related restriction logic 112 may receive an indicator from the server 110 that indicates whether the MCC of the merchant corresponds to at least one of the healthcare-related MCCs 126.

In both of the examples mentioned above, the MCC of the merchant corresponding to at least one of the healthcare-related MCCs 126 may indicate that the purchase transaction is to be executed (e.g., execution is to be triggered). Whereas, the MCC of the merchant not corresponding to at least one of the healthcare-related MCCs 126 may indicate that the purchase transaction is not to be executed (e.g., execution is not to be triggered). In one aspect, the MCC of the merchant may correspond to a healthcare-related MCC by semantically matching (i.e., sharing a common meaning with) the healthcare-related MCC. In another aspect, the MCC of the merchant may correspond to a healthcare-related MCC by being the same as the healthcare-related MCC.

In further accordance with this implementation, the healthcare-related restriction logic 112 selectively triggers execution of the purchase transaction depending on (e.g., based at least in part on) whether the MCC of the merchant corresponds to at least one of the healthcare-related MCCs. For example, the healthcare-related restriction logic 112 may trigger execution of the purchase transaction in response to (e.g., based at least in part on) the MCC of the merchant corresponding to at least one of the healthcare-related MCCs 126 by executing the purchase transaction or by instructing the POS terminal 104 to execute the purchase transaction. For instance, the healthcare-related restriction logic 112 may provide an execution instruction to the POS terminal 104 that causes the POS terminal 104 to execute the purchase transaction. In another example, the healthcare-related restriction logic 112 may not trigger execution of the purchase transaction in response to (e.g., based at least in part on) the MCC of the merchant not corresponding to at least one of the healthcare-related MCCs 126 by not executing the purchase transaction and by not instructing the POS terminal 104 to execute the purchase transaction.

In some example embodiments, the healthcare-related restriction logic 112 may selectively trigger execution of the purchase transaction further depending on whether one or more additional criteria are satisfied. For instance, the healthcare-related restriction logic 112 may selectively trigger execution of the purchase transaction further depending on whether the stock keeping unit (SKU) associated with the item corresponds to at least one pre-approved SKU. For example, a pre-approved SKU may be a SKU that is approved prior to the account identifier (e.g., account identifier 132) being provided to the point-of-sale terminal (e.g., POS terminal 104) from which the purchase request is received and prior to the purchase request being received from the point-of-sale.

In an example embodiment, the healthcare-related restriction logic 112 causes the SKU associated with the item to be cross-referenced with pre-approved SKUs. For example, the healthcare-related restriction logic 112 may cross-reference the SKU associated with the item with pre-approved SKUs, which may be stored in a store 116 of the server 110. In accordance with this example, the healthcare-related restriction logic 112 may retrieve the pre-approved SKUs from the server 110 (e.g., via the POS terminal 104) for comparison with the SKU associated with the item. In another example, the healthcare-related restriction logic 112 may instruct the server 110 to cross-reference the SKU associated with the item with the pre-approved SKUs. In accordance with this example, the healthcare-related restriction logic 112 may forward the SKU of the item to the server 110 (e.g., via the POS terminal 104) along with an instruction, which instructs the server 110 to cross-reference the SKU of the item with the pre-approved SKUs. In further accordance with this example, the healthcare-related restriction logic 112 may receive an indicator from the server 110 that indicates whether the SKU of the item corresponds to at least one of the pre-approved SKUs.

In both of the examples mentioned above, the SKU of the item corresponding to at least one of the pre-approved SKUs may indicate that the purchase transaction is to be executed (e.g., execution is to be triggered). Whereas, the SKU of the item not corresponding to at least one of the pre-approved SKUs may indicate that the purchase transaction is not to be executed (e.g., execution is not to be triggered), even if the MCC of the merchant corresponds to at least one of the healthcare-related MCCs 126. In one aspect, the SKU of the item may correspond to a pre-approved SKU by semantically matching (i.e., sharing a common meaning with) the pre-approved SKU. In another aspect, the SKU of the item may correspond to a pre-approved SKU by being the same as the pre-approved SKU.

For example, the healthcare-related restriction logic 112 may trigger execution of the purchase transaction in response to (e.g., based at least in part on) the SKU of the item corresponding to at least one pre-approved SKU and further in response to the MCC of the merchant corresponding to at least one of the healthcare-related MCCs 126 by executing the purchase transaction or by instructing the POS terminal 104 to execute the purchase transaction. In another example, the healthcare-related restriction logic 112 may not trigger execution of the purchase transaction in response to (e.g., based at least in part on) the SKU of the item not corresponding to at least one pre-approved SKU, even if the MCC of the merchant corresponds to at least one of the healthcare-related MCCs 126, by not executing the purchase transaction and by not instructing the POS terminal 104 to execute the purchase transaction.

In some example embodiments, the restricted-access credit device 102 and/or the store 116 may store a policy that identifies the healthcare-related MCCs. The healthcare-related restriction logic 112 may enforce the policy by selectively triggering execution of the purchase transaction depending on whether the MCC of the merchant corresponds to at least one of the healthcare-related MCCs.

The healthcare-related restriction logic 112 may be implemented in various ways to restrict access to the line of credit associated with the restricted-access credit device 102 including being implemented in hardware, software, firmware, or any combination thereof. For example, at least a portion of the healthcare-related restriction logic 112 may be implemented as computer program code configured to be executed in one or more processors. In another example, at least a portion of the healthcare-related restriction logic 112 may be implemented as hardware logic/electrical circuitry. For instance, at least a portion of the healthcare-related restriction logic 112 may be implemented in a field-programmable gate array (FPGA), an application-specific integrated circuit (ASIC), an application-specific standard product (ASSP), a system-on-a-chip system (SoC), a complex programmable logic device (CPLD), etc. Each SoC may include an integrated circuit chip that includes one or more of a processor (e.g., a microcontroller, microprocessor, digital signal processor (DSP), etc.), memory, one or more communication interfaces, and/or further circuits and/or embedded firmware to perform its functions.

The item scanner 108 is configured to scan an identifier associated with each item that is to be purchased. For instance, the identifier associated with each item may indicate (e.g., include) a SKU. The item scanner 108 is configured to generate item identification information 120 based at least in part on the identifier associated with each item. The item identification information 120 for each item indicates the item or a type of the item. For instance, the identification information may uniquely identify the item or the type of the item. The item identification information 120 may include the identifier or be derived from the identifier. The item scanner 108 may include an optical scanner and/or a tactile scanner.

In an example implementation, the item scanner 108 includes a light source, a lens, and a light sensor. The light source is configured to generate light to illuminate the identifier associated with each item. The lens is configured to direct the light that is reflected from each identifier toward the light sensor. For instance, the lens may focus the light on the light sensor. The light sensor is configured to translate the light that is received from the lens into an electrical signal.

In another example implementation, the identifier associated with each item includes a barcode, and the item scanner 108 includes a barcode scanner that is configured to read each barcode. For instance, the barcode scanner may shine light on each barcode and translate the light that is reflected from the barcode into an electrical signal that represents the item identification information 120. In accordance with this implementation, the item scanner 108 may be capable of decoding data that is included in each barcode to generate the item identification information 120.

The cash register 106 is configured to determine an amount to charge for each item that is to be purchased. For instance, the cash register 106 may cross-reference the item identification information 120 for each item with pre-determined item information associated with respective types of items. For example, the pre-determined item information may be stored in a database, and the cash register 106 may access the database to cross-reference the item identification information 120 for each item with the pre-determined item information associated with the respective types of items. The pre-determined item information for each type of item may identify the type of item and a cost associated with the type of item. For instance, each row of data in the database may correspond to a respective type of item. A first column of data in the database may include identifiers (e.g., names) of the respective types of items with which the respective rows correspond. A second column of data in the database may include per unit costs of the respective types of items with which the respective rows correspond. The cash register 106 may generate the cost information 124 for each item that is to be purchased to indicate the cost associated with the type of the item with which the item identification information 120 for the item corresponds. The cash register 106 may generate the item identifier 122 for each item based at least in part on the item identification information 120 for the respective item. For instance, the item identifier 122 for each item may include the item identification information 120 for the respective item or may be derived from the item identification information 120 for the respective item.

The POS terminal 104 is a processing system that is capable of communicating with the restricted-access credit device 102. The POS terminal 104 is configured to generate the purchase request 130, which includes the MCC of the merchant that is associated with the POS terminal 104. The POS terminal 104 may provide the item identifier 122 or information derived therefrom for each item that is to be purchased to the restricted-access credit device 102, though the scope of the example embodiments is not limited in this respect. The POS terminal 104 includes a display 114, which is configured to display information regarding each item that is to be purchased. For instance, the display 114 may display the item identifier 122 and the corresponding cost information 124 for each item that is to be purchased. The display 114 may further display a cumulative cost for the items that are to be purchased. The POS terminal 104 is configured to generate the purchase request based at least in part on the cumulative cost for the items that are to be purchased and the MCC of the merchant that is associated with the POS terminal 104.

The POS terminal 104 receives the account identifier 132 that identifies the line of credit associated with the restricted-access credit device 102 from the restricted-access credit device 102. The POS terminal 104 selectively applies the cumulative cost against the line of credit depending on (e.g., based at least in part on) whether the restricted-access credit device 102 triggers execution of the purchase transaction. The POS terminal 104 may selectively apply the cumulative cost against the line of credit further depending on whether the available credit associated with the line of credit is greater than or equal to the cumulative cost for the items. For instance, the POS terminal 104 may access (e.g., retrieve) available credit information 128, which indicates the amount of available credit associated with the line of credit, from the server 110 to determine whether the available credit associated with the line of credit is greater than or equal to the cumulative cost for the items. It will be recognized that the POS terminal 104 may include the cash register 106 and/or the item scanner 108, though the scope of the example embodiments is not limited in this respect.

The server 110 includes the store 116, which stores the healthcare-related MCCs 126 and the available credit information 128. The server 110 is configured to provide access to the healthcare-related MCCs 126 and the available credit information 128 in response to request for access that are received from the POS terminal 104. One type of store is a database. For instance, store 116 may be a relational database, an entity-relationship database, an object database, an object relational database, an extensible markup language (XML) database, etc.

It will be recognized that the restricted-access credit system 100 may not include all of the components shown in FIG. 1. Furthermore, the restricted-access credit system 100 may include components in addition to or in lieu of those shown in FIG. 1.

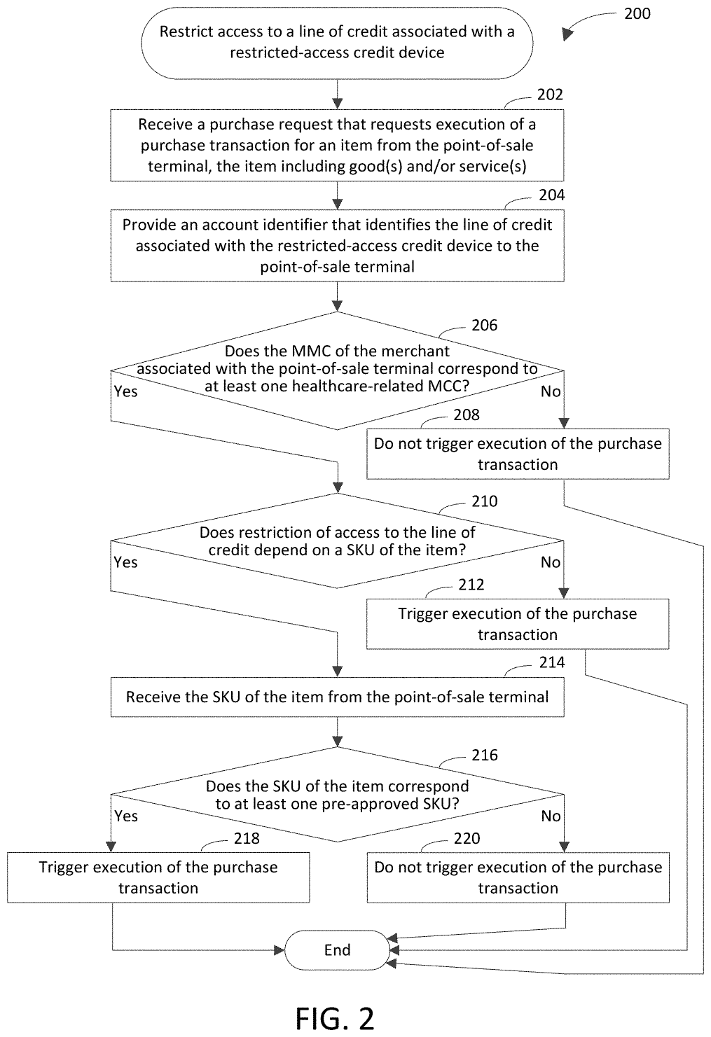

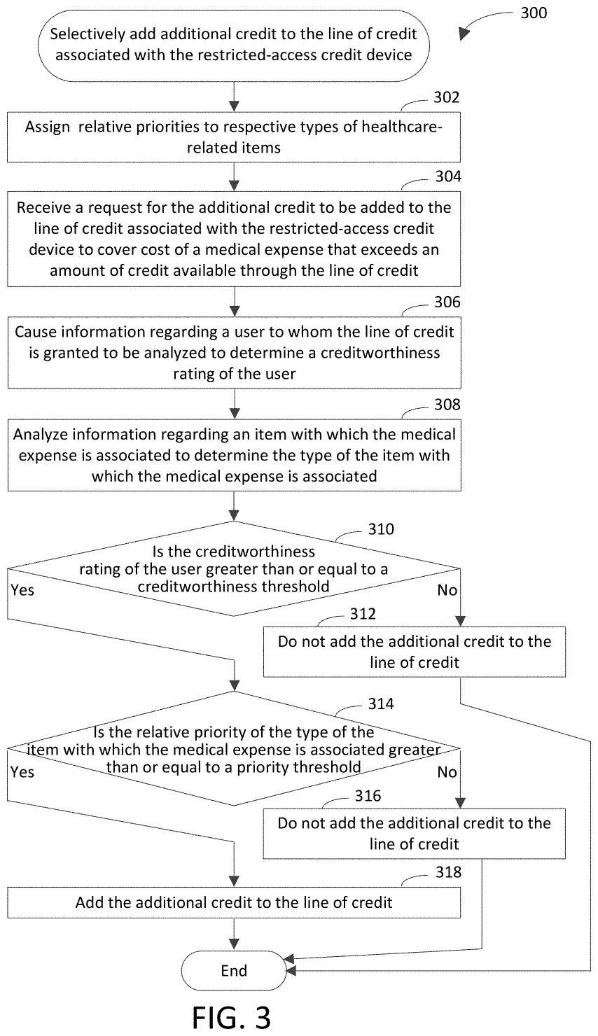

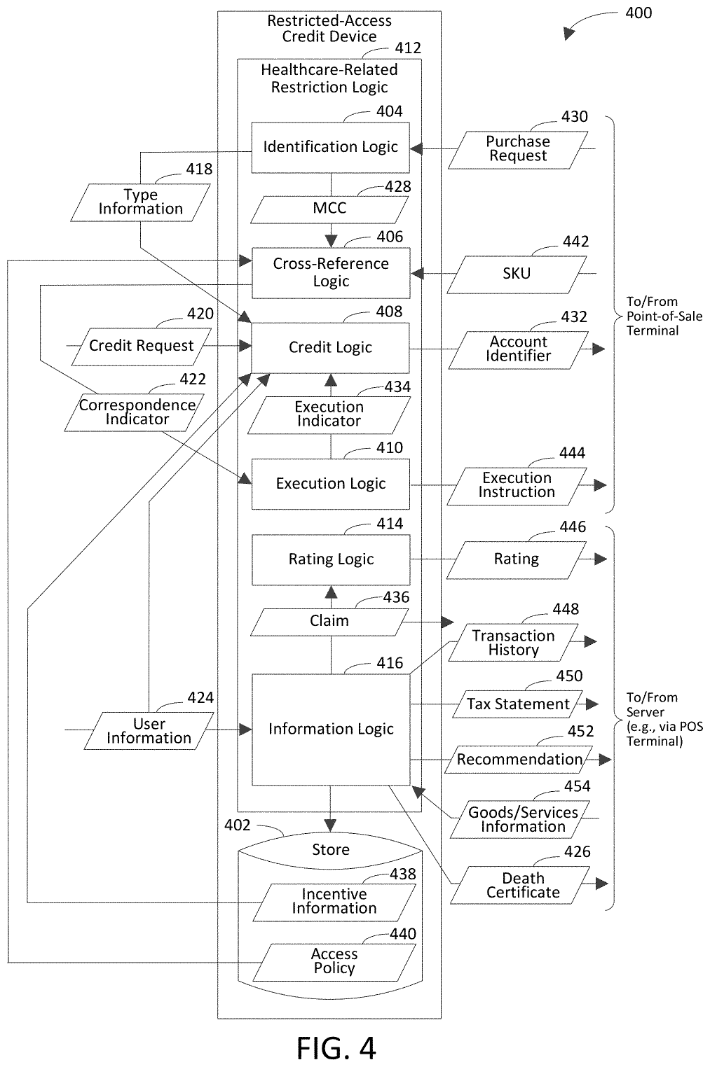

FIG. 2 depicts a flowchart 200 of an example method for restricting access to a line of credit associated with a restricted-access credit device in accordance with an embodiment. FIG. 3 depicts a flowchart 300 of an example method for selectively adding additional credit to a line of credit associated with a restricted-access credit device in accordance with an embodiment. Flowcharts 200 and 300 may be performed by the restricted-access credit device 102 shown in FIG. 1, for example. For illustrative purposes, flowcharts 200 and 300 are described with respect to the restricted-access credit device 400 shown in FIG. 4, which is an example implementation of the restricted-access credit device 102, according to an embodiment. As shown in FIG. 4, the restricted-access credit device 400 includes healthcare-related restriction logic 412 and a store 402. The healthcare-related restriction logic 412 includes identification logic 404, cross-reference logic 406, credit logic 408, execution logic 410, rating logic 414, and information logic 416. Further structural and operational embodiments will be apparent to persons skilled in the relevant art(s) based on the discussion regarding flowcharts 200 and 300.

As shown in FIG. 2, the method of flowchart 200 begins at step 202. In step 202, a purchase request that requests execution of a purchase transaction for an item is received from the point-of-sale terminal. The item includes good(s) and/or service(s). In an example implementation, the identification logic 404 receives a purchase request 430, which requests for the purchase transaction to be executed, from the point-of-sale terminal (e.g., POS terminal 104). In accordance with this example, the identification logic 404 identifies a merchant category code (MCC) 428 of a merchant that is associated with the point-of-sale terminal by analyzing the purchase request 430. The identification logic 404 may provide the MCC 428 of the merchant to the cross-reference logic 406 for processing.

At step 204, an account identifier that identifies the line of credit associated with the restricted-access credit device is provided to the point-of-sale terminal. In an example implementation, the credit logic 408 provides an account identifier 432 that identifies the line of credit associated with the restricted-access credit device 400 to the point-of-sale terminal. For example, the credit logic 408 may provide the account identifier 432 to the point-of-sale terminal in response to receipt of an execution indicator 434 from the execution logic 410. In accordance with this example, the execution indicator 434 may indicate whether the purchase transaction is to be executed. For instance, if the execution indicator 434 indicates that the purchase transaction is to be executed, the credit logic 408 may provide the account identifier 432 to the point-of-sale. If the execution indicator 434 indicates that the purchase transaction is not to be executed, the credit logic 408 may not provide the account identifier 432 to the point-of-sale. In another example, the credit logic 408 may provide the account identifier 432 to the point-of-sale terminal regardless whether the purchase transaction is to be executed.

At step 206, a determination is made whether the MCC of the merchant associated with the point-of-sale terminal corresponds to at least one healthcare-related MCC. If the MCC of the merchant corresponds to at least one healthcare-related MCC, flow continues to step 210. Otherwise, flow continues to step 208. In an example implementation, the cross-reference logic 406 determines whether the MCC 428 of the merchant corresponds to at least one healthcare-related MCC. For instance, the cross-reference logic 406 may cause the MCC 428 of the merchant to be cross-referenced with healthcare-related MCCs to determine whether the MCC 428 of the merchant corresponds to at least one healthcare-related MCC. A store (e.g., the store 402 or a store that is located remotely from the restricted-access credit device 400) may store the healthcare-related MCCs. In one example, the cross-reference logic 406 may access the store to identify the healthcare-related MCCs. In accordance with this example, the cross-reference logic 406 may cross-reference the MCC 428 of the merchant with the healthcare-related MCCs to determine whether the MCC 428 of the merchant corresponds to at least one of the healthcare-related MCCs. In another example, the cross-reference logic 406 may provide an instruction to an entity (e.g., a server) that is external (e.g., located remotely from) the restricted-access credit device. In accordance with this example, the instruction instructs the entity to cross-reference the MCC 428 of the merchant with the healthcare-related MCCs. In further accordance with this example, the cross-reference logic 406 may receive correspondence information from the entity that indicates whether the MCC 428 of the merchant corresponds to at least one of the healthcare-related MCCs. In accordance with this implementation, the cross-reference logic 406 may generate a correspondence indicator 422 to indicate whether the MCC 428 of the merchant corresponds to at least one of the healthcare-related MCCs. For instance, the cross-reference logic 406 may generate the correspondence indicator 422 based on (e.g., based at least in part on) its own cross-referencing of the MCC 428 of the merchant with the healthcare-related MCCs or based on the correspondence information received from the entity.

At step 208, execution of the purchase transaction is not triggered. For example, the purchase transaction may not be triggered based at least in part on the MCC of the merchant not corresponding to at least one healthcare-related MCC. In another example, not triggering execution of the purchase transaction may include denying the request to execute the purchase transaction. Upon completion of step 208, flowchart 200 ends. In an example implementation, the execution logic 410 does not trigger execution of the purchase transaction. For example, the execution logic 410 may not trigger execution of the purchase transaction in response to receipt of the correspondence indicator 422. In accordance with this example, the execution logic 410 may not trigger execution of the purchase transaction based at least in part on the correspondence indicator 422 indicating that the MCC 428 of the merchant does not correspond to at least one of the healthcare-related MCCs.

At step 210, a determination is made whether restriction of access to the line of credit depends on a SKU of the item. If the restriction of the access depends on the SKU of the item, flow continues to step 214. Otherwise, flow continues to step 212. In an example implementation, the cross-reference logic 406 determines whether the restriction of the access to the line of credit depends on a SKU 442 of the item. For example, the store 402 may store an access policy 440 defining how access to the line of credit is to be restricted. The access policy 440 may include rules that dictate whether the restriction of the access to the line of credit depends on the SKU of the item. The cross-reference logic 406 may retrieve the access policy 440 from the store 402 for review. The cross-reference logic 406 may determine whether the restriction of the access to the line of credit depends on the SKU of the item by analyzing the rules in the access policy 440 that define how access to the line of credit is to be restricted. It will be recognized that the cross-reference logic 406 may review the access policy 440 to determine whether the restriction of the access to the line of credit depends on the MCC 428 of the merchant prior to determining whether the MCC 428 of the merchant corresponds to at least one healthcare-related MCC at step 206, though the scope of the example embodiments is not limited in this respect.

At step 212 execution of the purchase transaction is triggered. For example, the purchase transaction may be triggered based at least in part on the MCC of the merchant corresponding to at least one healthcare-related MCC. In accordance with this example, the purchase transaction may be triggered further based at least in part on the restriction of the access to the line of credit not depending on the SKU of the item. Upon completion of step 212, flowchart 200 ends. In an example implementation, the execution logic 410 triggers execution of the purchase transaction. For example, the execution logic 410 may trigger execution of the purchase transaction in response to receipt of the correspondence indicator 422. In accordance with this example, the execution logic 410 may trigger execution of the purchase transaction based at least in part on the correspondence indicator 422 indicating that the MCC 428 of the merchant corresponds to at least one of the healthcare-related MCCs. In further accordance with this example, the execution logic 410 may trigger execution of the purchase transaction further based at least in part on the correspondence indicator 422 indicating that the restriction of the access to the line of credit does not depend on the SKU 442 of the item.

At step 214, the SKU of the item is received from the point-of-sale terminal. The SKU is configured to distinguish an item type of the item from other item types of other items. In an example implementation, the cross-reference logic 406 receives the SKU 442 of the item from the point-of-sale terminal.

At step 216, a determination is made whether the SKU of the item corresponds to at least one pre-approved SKU. For example, each pre-approved SKU may have been pre-approved because it is a healthcare-related SKU. In accordance with this example, SKUs that are healthcare-related may be pre-approved; whereas, SKUs that are not healthcare-related may not be pre-approved. If the SKU of the item corresponds to at least one pre-approved SKU, flow continues to step 218. Otherwise, flow continues to step 220. In an example implementation, the cross-reference logic 406 determines whether the SKU 442 of the item corresponds to at least one pre-approved SKU. For instance, the cross-reference logic 406 may cause the SKU 442 of the item to be cross-referenced with pre-approved SKUs to determine whether the SKU 442 of the item corresponds to at least one pre-approved SKU. A store (e.g., the store 402 or a store that is located remotely from the restricted-access credit device 400) may store the pre-approved SKUs. In one example, the cross-reference logic 406 may access the store to identify the pre-approved SKUs. In accordance with this example, the cross-reference logic 406 may cross-reference the SKU 442 of the item with the pre-approved SKUs to determine whether the SKU 442 of the item corresponds to at least one of the pre-approved SKUs. In another example, the cross-reference logic 406 may provide an instruction to an entity (e.g., a server) that is external (e.g., located remotely from) the restricted-access credit device. In accordance with this example, the instruction instructs the entity to cross-reference the SKU 442 of the item with the pre-approved SKUs. In further accordance with this example, the cross-reference logic 406 may receive correspondence information from the entity that indicates whether the SKU 442 of the item corresponds to at least one of the pre-approved SKUs. In accordance with this implementation, the cross-reference logic 406 may generate a correspondence indicator 422 to indicate whether the SKU 442 of the item corresponds to at least one of the pre-approved SKUs. For instance, the cross-reference logic 406 may generate the correspondence indicator 422 based on (e.g., based at least in part on) its own cross-referencing of the SKU 442 of the item with the pre-approved SKUs or based on the correspondence information received from the entity.

At step 218, execution of the purchase transaction is triggered. For example, the purchase transaction may be triggered based at least in part on the SKU of the item corresponding to at least one pre-approved SKU and further based at least in part on the MCC of the merchant corresponding to at least one healthcare-related MCC. Upon completion of step 218, flowchart 200 ends. In an example implementation, the execution logic 410 triggers execution of the purchase transaction. For example, the execution logic 410 may trigger execution of the purchase transaction in response to receipt of the correspondence indicator 422. In accordance with this example, the execution logic 410 may trigger execution of the purchase transaction based at least in part on the correspondence indicator 422 indicating that the SKU 422 of the item corresponds to at least one pre-approved SKU and further based at least in part on the correspondence indicator 422 indicating that the MCC 428 of the merchant corresponds to at least one of the healthcare-related MCCs.

At step 220, execution of the purchase transaction is not triggered. For instance, the purchase transaction may not be triggered based at least in part on the SKU of the item not corresponding to at least one pre-approved SKU (e.g., even though the MCC of the merchant corresponds to at least one healthcare-related MCC). Upon completion of step 220, flowchart 200 ends. In an example implementation, the execution logic 410 does not trigger execution of the purchase transaction. For example, the execution logic 410 may not trigger execution of the purchase transaction in response to receipt of the correspondence indicator 422. In accordance with this example, the execution logic 410 may not trigger execution of the purchase transaction based at least in part on the correspondence indicator 422 indicating that the SKU 422 of the item does not correspond to at least one pre-approved SKU (e.g., even though the correspondence indicator 422 indicates that the MCC 428 of the merchant corresponds to at least one of the healthcare-related MCCs).

In an example embodiment, the restricted-access credit device is linked to a tax-advantaged health benefit account of a user to whom the line of credit is granted. A tax-advantaged health benefit account is a health benefit account that qualifies for a reduced tax liability, a deferred tax liability (e.g., deferred from a time at which funds are deposited into the health benefit account), or no tax liability. A health benefit account is an account into which tax-reduced, tax-deferred, or tax-free amounts from income of a user are capable of being deposited for purposes of healthcare of the user. Examples of a tax-advantaged health benefit account include but are not limited to a health savings account (HSA), a flexible spending account (FSA), a Medicare account, and a Medicaid account. In accordance with this embodiment, the tax-advantaged health benefit account has funds available for purchases (e.g., via a revolving line of credit that is applicable toward healthcare purchases). In an aspect of this embodiment, triggering execution of the purchase transaction at step 212 or step 218 includes causing the funds of the tax-advantaged health benefit account to be applied toward a cost of the item. In accordance with this aspect, triggering execution of the purchase transaction at step 212 or step 218 may further include determining that the cost exceeds the funds by an amount and causing the amount to be applied against the line of credit. Accordingly, the amount may be paid using (e.g., from) the line of credit. In another aspect of this embodiment, triggering execution of the purchase transaction at step 212 or step 218 includes causing the funds of the tax-advantaged health benefit account to be applied toward the cost of the item in monthly installments.

In some example embodiments, one or more steps 202, 204, 206, 208, 210, 212, 214, 216, 218, and/or 220 of flowchart 200 may not be performed. Moreover, steps in addition to or in lieu of steps 202, 204, 206, 208, 210, 212, 214, 216, 218, and/or 220 may be performed. For instance, in an example embodiment, the method of flowchart 200 further includes establishing a communication channel (e.g., a secure communication channel) with the point-of-sale terminal. For instance, the execution logic 410 may establish the communication channel between the restricted-access credit device 400 and the POS terminal 104. In accordance with this embodiment, providing the account identifier at step 204 includes providing the account identifier via the communication channel.

In another example embodiment, execution of the purchase transaction is triggered at step 212 or 218 by causing a cost of the item to be applied against the line of credit. For instance, causing the cost of the item to be applied against the line of credit may include causing the cost to be paid using (e.g., from) the line of credit. In accordance with this embodiment, information regarding an incentive offered by an employer of a user to whom the line of credit is granted may be stored. For instance, the store 402 may store incentive information 438, which includes the information regarding the incentive. The incentive offers a credit based at least in part on a purchase of the item from the merchant that is associated with the point-of-sale terminal rather than from another merchant that offers the item for sale.

In accordance with this embodiment, the credit may be applied to the line of credit, toward an insurance deductible of the user, and/or toward out-of-pocket healthcare expenses of the user based at least in part on the user purchasing the item from the merchant rather than from another merchant that offers the item for sale. In one example, the credit may be a discount (e.g., 5%, 10%, or 15% discount) associated with the item. In another example, the credit is in the form of reward points. For instance, a number of the reward points may be proportional to the cost of the item (e.g., 3 points per dollar of the cost or 5 points per dollar of the cost). The rewards may serve as an incentive for the user to use the restricted-access credit device. The user may donate the rewards to a charitable medical organization.

In yet another example embodiment, the method of flowchart 200 further includes downloading a transaction history, which identifies items purchased with the restricted-access credit device, from a memory that is remote from the restricted-access credit device to the memory that is included in the restricted-access credit device. For instance, the memory from which the transaction history is downloaded may be coupled to the restricted-access credit device through a network. In an example implementation, the information logic 416 downloads transaction history 448, which identifies the items purchased, from the memory that is remote from the restricted-access credit device 400 to the store 402. In an aspect of this embodiment, the method of flowchart 200 further includes preparing (e.g., automatically preparing) an annual tax statement of a user to whom the line of credit is granted based at least in part on the transaction history. For instance, the information logic 416 may prepare a tax statement 450 of the user based at least in part on the transaction history 448.

In still another example embodiment, the method of flowchart 200 further includes providing indemnity up to a designated amount for an ambulance trip, an overnight hospital stay, a disease screening, and/or an emergency room visit based at least in part on payment of a membership fee to use the restricted-access credit device by a user to whom the line of credit is granted. In an example implementation, the credit logic 408 provides the indemnity. For instance, the credit logic 408 may automatically cause the designated amount to be reimbursed to the line of credit in response to the designated amount being applied against the line of credit for the ambulance trip, the overnight hospital stay, the disease screening, and/or the emergency room visit.

In another example embodiment, the method of flowchart 200 further includes providing indemnity up to a first designated amount for an ambulance trip, an overnight hospital stay, a disease screening, and/or an emergency room visit in a first geographic region in which a user to whom the line of credit is granted resides based at least in part on payment of a first membership fee to use the restricted-access credit device (e.g., card) by the user. In accordance with this embodiment, the method of flowchart 200 further includes providing indemnity up to a second designated amount for the ambulance trip, the overnight hospital stay, the disease screening, and/or the emergency room visit in a second geographic region that is different from the first geographic region based at least in part on payment of a second membership fee that is greater than the first membership fee by the user. In an example implementation, the credit logic 408 provides the indemnity up to the first designated amount in the first geographical region and the indemnity up to the second designated amount in the second geographical region. The of the first and second geographical regions may be a city, state, country, continent, or other suitable geographical region. The first designated amount and the second designated amount may be the same or different.

In yet another example embodiment, the method of flowchart 200 further includes applying (e.g., automatically applying) an insurance deductible associated with a health insurance policy of a user to whom the line of credit is granted and/or a co-payment associated with the health insurance policy against the line of credit. For instance, the insurance deductible and/or the co-payment may be paid using (e.g., from) the line of credit. In an example implementation, the credit logic 408 applies the insurance deductible and/or the co-payment against the line of credit.

In still another example embodiment, the restricted-access credit device is linked to a tax-advantaged health benefit account of a user to whom the line of credit is granted. In accordance with this embodiment, the method of flowchart 200 includes providing monthly payments from the tax-advantaged health benefit account toward a standing balance resulting from charges applied against the line of credit. In an example embodiment, the credit logic 408 provides the monthly payments from the tax-advantaged health benefit account toward the standing balance.

In another example embodiment, the method of flowchart 200 further includes selecting one or more healthcare-related items, which are to be recommended to a user to whom the line of credit is granted, from healthcare-related items based on any of a variety of factors. Examples of such a factor include but are not limited to a purchasing behavior (e.g., transaction history 448) of the user, a medical history of the user, and a location of the user. For example, the purchasing behavior of the user may indicate healthcare-related items (e.g., products and services) that the user has purchased in the past, a frequency with which each of the healthcare-related items have been purchased, a cost of each of the healthcare-related items, dates on which the healthcare-related items were purchased, and facilities (e.g., pharmacies, emergency centers, hospitals, diagnostic facilities, and wellness clinics) at which the items were purchased. Examples of a healthcare-related product include but are not limited to a book regarding a healthcare-related topic, electronic content (e.g., access to a downloadable file, a computer-readable medium that stores such a file) pertaining to a healthcare-related topic, and a medication. Examples of a healthcare-related service include but are not limited to a medical test, a surgery, a chiropractic treatment, a psychiatric treatment, a dental treatment, and a medical check-up. The medical history of the user may indicate medical tests, surgeries, and check-ups that have been performed on the user, the dates on which they occurred, and the results; medications that the user has taken, dates on which the medications were taken, doses of the medications, mediums through which the medications were taken (e.g., intravenously, orally, topically); medical conditions (e.g., diseases) that the user or the user's family (e.g., ancestors) currently has or has had in the past. The location of the user may be a city, state, and/or country in which the user is presently located or in which the user resides.

In accordance with this embodiment, the information logic 416 may select the one or more healthcare-related items. For example, the information logic 416 may analyze any one or more of the factors mentioned above to determine which of the healthcare-related items to select. In accordance with this example, analysis of the factors may reveal a health issue or potential health issue that may be avoided, mitigated, or resolved by identified healthcare-related item(s). Accordingly, the information logic 416 may select the identified healthcare-related item(s) to be included among the one or more healthcare-related items that are to be recommended to the user.

In further accordance with this embodiment, the one or more healthcare-related items are recommended to the user based at least in part on selection of the one or more healthcare-related items. For instance, the information logic 416 may provide a recommendation 452 to the user that recommends the one or more healthcare-related items. The user may opt-in or opt-out of receiving such recommendations.

In yet another example embodiment, the method of flowchart 200 further includes selecting one or more healthcare-related facilities, which are to be recommended to a user to whom the line of credit is granted, from healthcare-related facilities based at least in part on a quality rating of each of the one or more healthcare-related facilities, a cost of one or more services at each of the one or more healthcare-related facilities, and/or a location of each of the one or more healthcare-related facilities.

In accordance with this embodiment, the information logic 416 may select the one or more health facilities. In accordance with this example, the information logic 416 may analyze goods/services information 454 to determine which of the healthcare-related facilities to select. In a first aspect, the information logic 416 may compare the quality ratings of the respective healthcare-related facilities, as identified in the goods/services information 454, to a quality threshold to determine a first subset of the healthcare-related facilities in which each healthcare-related facility has a quality rating that is greater than or equal to the quality threshold. In a second aspect, the information logic 416 may compare the cost of the one or more services at each of the healthcare-related facilities, as identified in the goods/services information 454, to determine a second subset of the healthcare-related facilities in which each healthcare-related facility offers a cost that is less than or equal to the cost offered by each healthcare-related facility that is not included in the second subset. In accordance with this aspect, the information logic 416 may define the second subset to include approximately a designated percentage of the healthcare-related facilities or to include each healthcare-related facility that offers a cost that is less than or equal to a cost threshold. In a third aspect, the information logic 416 may analyze the goods/services information 454 to determine a location of each of the healthcare-related facilities. In accordance with this aspect, the information logic 416 may determine a distance between a location of the user and a location of each of the healthcare-related facilities. In further accordance with this aspect, the information logic 416 may compare the distances between the location of the user and the respective healthcare-related facilities to a distance threshold to determine a third subset of the healthcare-related facilities such that the distance between the location of the user and the location of each healthcare-related facility in the third subset is less than or equal to the distance threshold.

In further accordance with this embodiment, the method of flowchart 200 further includes recommending the one or more healthcare-related facilities to the user based at least in part on selection of the one or more healthcare-related facilities. For instance, the information logic 416 may recommend the one or more healthcare-related facilities to the user.

In still another example embodiment, the item includes a medical procedure performed on a user to whom the line of credit is granted. In accordance with this embodiment, the method of flowchart 200 further includes causing a claim regarding medical misconduct and/or medical malpractice to be generated on behalf of the user based at least in part on information regarding damages that the user claims to have occurred as a result of the medical procedure, healthcare data of the user, and/or information regarding the purchase transaction. In an example implementation, the information logic 416 causes a claim 436 regarding medical misconduct and/or medical malpractice to be generated on behalf of the user based at least in part on an analysis of the user information 424 and/or the transaction history 448. For instance, the user information 424 may indicate damages that the user claims to have occurred as a result of the medical procedure. In another example, the user information 424 may include the healthcare data of the user. In yet another example, the transaction history 448 may include the information regarding the purchase transaction. The information logic 416 may be configured to enable the user to contact an administrator of the restricted-access credit device 400 to notify the administrator of the alleged medical misconduct and/or medical malpractice. The credit logic 408 may be capable of remediating an issue regarding a charge for the item against the line of credit in response to the claim 436 being generated or being resolved in the user's favor.

In accordance with this embodiment, the method of flowchart 200 may further include generating a rating for a medical facility at which the medical procedure is performed and/or a doctor who performed the medical procedure based at least in part on the claim. Accordingly, the rating may be generated to take into consideration the claim. For instance, the rating logic 414 may generate a rating 446 for the medical facility and/or the doctor based at least in part on the claim 436. For instance, if the claim is successful, the rating of the facility and/or the doctor may be reduced by an incremental amount. If the claim is not successful, the rating of the facility and/or the doctor may remain unchanged or may be reduced by an amount that is less than the amount that the rating would have been reduced if the claim had been successful. The rating logic 414 may share the rating 446 with users of other restricted-access credit devices that are offered by a provider that offers the restricted-access credit device 400, though the example embodiments are not limited in this respect.

In another example embodiment, the method of flowchart 200 further includes processing (e.g., automatically processing) a death certificate of a user to whom the line of credit is granted. For example, the information logic 416 may process (e.g., generate) a death certificate 426 of the user. In accordance with this example, the information logic 416 may process the death certificate by analyzing the user information 424 to determine identifying information regarding the user and to determine that the user has died.

In yet another example embodiment, the method of flowchart 200 includes one or more of the steps shown in flowchart 300 of FIG. 3. As shown in FIG. 3, the method of flowchart 300 begins at step 302. In step 302, relative priorities are assigned to respective types of healthcare-related items. In an example implementation, the credit logic 408 assigns the relative priorities to the respective types of healthcare-related items. For instance, the credit logic 408 may assign a first priority to surgical procedures or a specified type of surgical procedure, a second priory to medications or a specified type of medication, a third priority to preventative tests or a specified type of preventative test, and so on. Each of the priorities may be different from the other priorities. The credit logic 408 may store the relative priorities in a store (e.g., the store 402 or a store that is external to the restricted-access credit device 400).

At step 304, a request for the additional credit to be added to the line of credit associated with the restricted-access credit device to cover cost of a medical expense that exceeds an amount of credit available through the line of credit is received. In an example implementation, the credit logic 408 receives a credit request 420 that requests for the additional credit to be added to the line of credit.

At step 306, information regarding a user to whom the line of credit is granted is caused to be analyzed to determine a creditworthiness rating of the user. For example, the information regarding the user may be automatically caused (e.g., in real-time) to be analyzed in response to receipt of the request. In another example, the information regarding the user may include information indicating creditworthiness of the user. In an example implementation, the credit logic 408 causes user information 424, which pertains to the user, to be analyzed to determine the creditworthiness rating of the user. For example, the credit logic 408 may analyze the user information 424. In accordance with this example, the credit logic 408 may retrieve the user information 424 from the store 402 or from a source that is external to the restricted-access credit device 400 for analysis. In another example, the credit logic 408 may instruct an entity that is external to the restricted-access credit device 400 to analyze the user information 424. In accordance with this example, the credit logic 408 may forward the user information 424 to the entity along with an instruction, which instructs the entity to analyze the user information 424 to determine the creditworthiness rating of the user. In further accordance with this example, the credit logic 408 may receive an indicator from the entity that indicates the creditworthiness rating of the user.

At step 308, information regarding an item with which the medical expense is associated is analyzed to determine the type of the item with which the medical expense is associated. In an example implementation, the identification logic 404 analyzes the information regarding the item to determine the type of the item. For instance, the information regarding the item may be included in the purchase request, and the identification logic 404 may review the information therein to determine the type of the item. In accordance with this implementation, the identification logic 404 may generate type information 418 to indicate the type of the item based at least in part on the analysis of the information.

At step 310, a determination is made whether the creditworthiness rating of the user is greater than or equal to a creditworthiness threshold. If the creditworthiness rating of the user is greater than or equal to the creditworthiness threshold, flow continues to 314. Otherwise, flow continues to step 312. In an example implementation, the credit logic 404 determines whether the creditworthiness rating of the user is greater than or equal to the creditworthiness threshold. For instance, may compare the creditworthiness rating of the user to the creditworthiness threshold to make the determination.

At step 312, the additional credit is not added to the line of credit. Upon completion of step 312, flowchart 300 ends. In an example implementation, the credit logic 408 does not add the additional credit to the line of credit (e.g., in response to the creditworthiness rating of the user not being greater than or equal to the creditworthiness threshold).

At step 314, a determination is made whether the relative priority of the type of the item with which the medical expense is associated is greater than or equal to a priority threshold. If the relative priority of the type of the item with which the medical expense is associated is greater than or equal to the priority threshold, flow continues to step 318. Otherwise, flow continues to step 316. In an example implementation, the credit logic 408 determines whether the relative priority of the type of the item is greater than or equal to the priority threshold. For example, the credit logic 408 may cross-reference the type of the item with the various types of healthcare-related items and their assigned relative priorities to determine the relative priority of the type of the item with which the medical expense is associated. In accordance with this example, the credit logic 408 may compare the relative priority of the type of the item with which the medical expense is associated to the priority threshold to determine whether the relative priority of the type of the item with which the medical expense is associated is greater than or equal to the priority threshold.

At step 316, the additional credit is not added to the line of credit. Upon completion of step 316, flowchart 300 ends. In an example implementation, the credit logic 408 does not add the additional credit to the line of credit (e.g., in response to the relative priority of the type of the item with which the medical expense is associated not being greater than or equal to the priority threshold). For instance, the credit logic 408 may not add the additional credit to the line of credit at step 316 even though the creditworthiness rating of the user is greater than or equal to the creditworthiness threshold.

At step 318, the additional credit is added to the line of credit. For instance, the additional credit may be automatically added to the line of credit (e.g., in real-time) in response to the determinations being made at steps 310 and 314. Upon completion of step 318, flowchart 300 ends. In an example implementation, the credit logic 408 adds the additional credit to the line of credit (e.g., in response to the creditworthiness rating of the user being greater than or equal to the creditworthiness threshold and/or in response to the relative priority of the type of the item with which the medical expense is associated being greater than or equal to the priority threshold). The credit logic 408 may add the additional credit to the line of credit with a restriction that the additional credit is to be used for a specified provider and/or a specified event. For instance, if the user requests the additional credit for heart surgery at City Medical Center, the restriction may require that the addition credit be used only for the heart surgery and/or only at the City Medical Center.

In an example embodiment, the information is caused to be analyzed at step 306 further to determine an extent to which the user needs the item with which the medical expense is associated. The extent to which the user needs the item may be measured by an extent to which the item is expected to improve the user's health or an amount of time that the item is expected to add to the user's lifespan. In accordance with this embodiment, the additional credit is added to the line of credit at step 318 based at least in part on the extent to which the user needs the item with which the medical expense is associated exceeding a need threshold.

In some example embodiments, one or more steps 302, 304, 306, 308, 310, 312, 314, 316, and/or 318 of flowchart 300 may not be performed. Moreover, steps in addition to or in lieu of steps 302, 304, 306, 308, 310, 312, 314, 316, and/or 318 may be performed.

It will be recognized that the restricted-access credit device 400 may not include one or more of the store 402, the identification logic 404, the cross-reference logic 406, the credit logic 408, the execution logic 410, the healthcare-related restriction logic 412, the rating logic 414, and/or the information logic 416. Furthermore, the restricted-access credit device 400 may include components in addition to or in lieu of store 402, the identification logic 404, the cross-reference logic 406, the credit logic 408, the execution logic 410, the healthcare-related restriction logic 412, the rating logic 414, and/or the information logic 416.

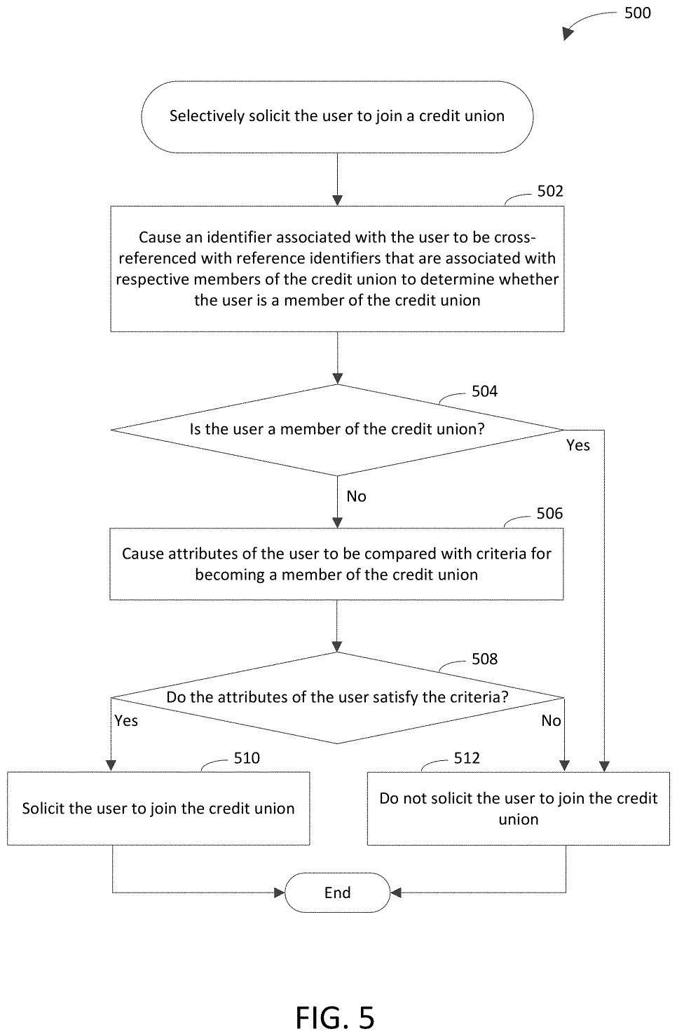

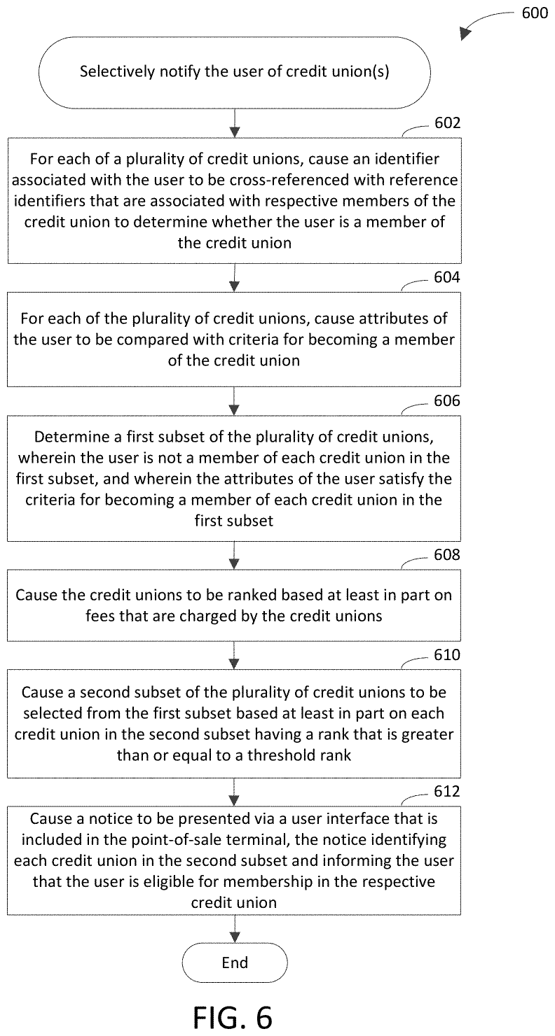

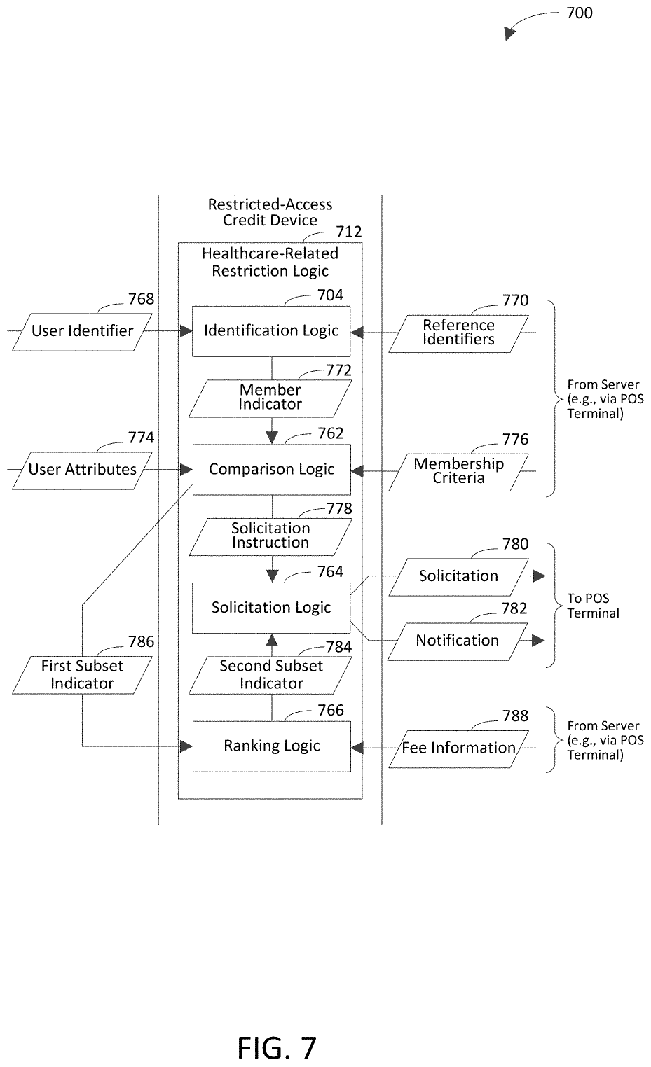

In still other example embodiments, the method of flowchart 200 includes one or more of the steps shown in flowchart 500 of FIG. 5 and/or one or more of the steps shown in flowchart 600 of FIG. 6. FIG. 5 depicts a flowchart 500 of an example method for selectively soliciting the user to join a credit union in accordance with an embodiment. FIG. 6 depicts a flowchart 600 of an example method for selectively notifying the user of credit union(s) in accordance with an embodiment. Flowcharts 500 and 600 may be performed by the restricted-access credit device 102 shown in FIG. 1, for example. For illustrative purposes, flowcharts 500 and 600 are described with respect to the restricted-access credit device 700 shown in FIG. 7, which is an example implementation of the restricted-access credit device 102, according to an embodiment. As shown in FIG. 7, the restricted-access credit device 700 includes healthcare-related restriction logic 712. The healthcare-related restriction logic 712 includes identification logic 704, comparison logic 762, solicitation logic 764, and ranking logic 766. Further structural and operational embodiments will be apparent to persons skilled in the relevant art(s) based on the discussion regarding flowcharts 500 and 600.

As shown in FIG. 5, the method of flowchart 500 begins at step 502. In step 502, an identifier associated with the user is caused to be cross-referenced with reference identifiers that are associated with respective members of the credit union to determine whether the user is a member of the credit union. The identifier matching at least one of the reference identifiers indicates that the user is a member of the credit union. The identifier not matching at least one of the reference identifiers indicates that the user is not a member of the credit union. The identifier associated with the user may be included in the account identifier that identifies the line of credit. In an example implementation, the identification logic 704 causes a user identifier 768 associated with the user to be cross-referenced with reference identifiers 770 that are associated with the respective members of the credit union. For example, the identification logic 704 may cross-reference the user identifier 768 with the reference identifiers 770. In another example, the identification logic 704 may instruct the server (e.g., a store included therein) to cross-reference the user identifier 768 with the reference identifiers 770. In accordance with this example, the identification logic 704 may receive an indicator from the server, indicating whether the user is a member of the credit union.

At step 504, a determination is made whether the user is a member of the credit union. If the user is a member of the credit union, flow continues to step 512. Otherwise, flow continues to step 506. In an example implementation, the identification logic 704 determines whether the user is a member of the credit union. For example, the identification logic 704 may make the determination based on its own cross-referencing of the user identifier 768 with the reference identifiers 770. In another example, the identification logic 704 may make the determination based on an indicator received from the store, indicating whether the user is a member of the credit union. In both of these examples, the identification logic 704 may generate a member indicator 772 to specify whether the user is a member of the credit union.

At step 506, attributes of the user are caused to be compared with criteria for becoming a member of the credit union. In an example implementation, the comparison logic 762 causes user attributes 774 of the user to be compared with membership criteria 776, which indicates criteria for becoming a member of the credit union. For instance, the comparison logic 762 may cause the user attributes 774 of the user to be compared with the membership criteria 776 based on the member indicator 772 indicating that the user is not a member of the credit union. In an example, the comparison logic 762 compares the user attributes 774 of the user to the membership criteria 776. In another example, the comparison logic 762 instructs the server to compare the user attributes 774 to the membership criteria 776. In accordance with this example, the comparison logic 762 may receive an indicator from the server, indicating whether the user attributes 774 satisfy the membership criteria 776.

At step 508, a determination is made whether the attributes of the user satisfy the criteria. If the attributes of the user satisfy the criteria, flow continues to step 510. Otherwise, flow continues to step 512. In an example implementation, the comparison logic 762 determines whether the user attributes 774 satisfy the membership criteria 776. For example, the comparison logic 762 may make the determination based on its own comparison of the user attributes 774 with the membership criteria 776. In another example, the comparison logic 762 may make the determination based on an indicator received from the server, indicating whether the user attributes 774 satisfy the membership criteria 776. The comparison logic 762 may be configured to generate a solicitation instruction 778 based on the user attributes 774 satisfying the membership criteria 776. The solicitation instruction 778 instructs the solicitation logic 764 to solicit the user to join the credit union. The comparison logic 762 may be configured to not generate the solicitation instruction 778 based on the user attributes 774 not satisfying the membership criteria 776.

At step 510, the user is solicited to join the credit union (e.g., via a user interface that is included in the point-of-sale terminal). It can be said that the user is solicited to join the credit union based on the user not being a member of the credit union and further based on the attributes of the user satisfying the criteria. In an example implementation, the solicitation logic 764 generates a solicitation 780, which solicits the user to join the credit union. For instance, the solicitation logic 764 may solicit the user to join the credit union based on receipt of the solicitation instruction 778. In accordance with this implementation, the solicitation logic 764 may cause the solicitation 780 to be presented via a user interface that is included in the point-of-sale terminal.

At step 512, the user is not solicited to join the credit union. It can be said that the user is not solicited to join the credit union based on the user being a member of the credit union and/or the attributes of the user not satisfying the criteria. In an example implementation, the solicitation logic 764 does not solicit the user to join the credit union. For instance, the solicitation logic 764 may not generate the solicitation 780 to solicit the user to join the credit union based on the solicitation instruction 778 not being received from the comparison logic 762.

As shown in FIG. 6, the method of flowchart 600 begins at step 602. In step 602, for each of a plurality of credit unions, an identifier associated with the user is caused to be cross-referenced with reference identifiers that are associated with respective members of the credit union to determine whether the user is a member of the credit union. The identifier matching at least one of the reference identifiers indicates that the user is a member of the credit union. The identifier not matching at least one of the reference identifiers indicates that the user is not a member of the credit union. In an example implementation, for each of the credit unions, the identification logic 704 causes the user identifier 768 associated with the user to be cross referenced with reference identifiers 770 that are associated with respective members of the credit union to determine whether the user is a member of the credit union. For instance, the identification logic 704 may cross-reference the user identifier 768 with the reference identifiers 770 for each credit union, or the identification logic 704 may instruct the server (e.g., a store therein) to do so. In accordance with this implementation, the identification logic 704 may generate the member indicator 772 to indicate whether the user is a member of each of the credit unions.

At step 604, for each of the plurality of credit unions, attributes of the user are caused to be compared with criteria for becoming a member of the credit union. In an example implementation, the comparison logic 762 causes user attributes 774 of the user to be compared with membership criteria 776 for each of the credit unions. The membership criteria 776 for each credit union specifies requirements for becoming a member of the credit union. For instance, the comparison logic 762 may compare the user attributes 774 of the user with the membership criteria 776 for each of the credit unions, or the comparison logic 762 may instruct the server to do so.

At step 606, a first subset of the plurality of credit unions is determined such that the user is not a member of each credit union in the first subset and the attributes of the user satisfy the criteria for becoming a member of each credit union in the first subset. In an example implementation, the comparison logic 762 determines the first subset of the credit unions. For example, the comparison logic 762 may make the determination based on its own comparison of the user attributes 774 to the membership criteria 776 for each of the credit unions. In another example, the comparison logic 762 may make the determination based on receipt of an indicator from the server, indicating whether the user attributes 774 of the user satisfy the membership criteria 776 for each of the credit unions. In accordance with this implementation, the comparison logic 762 may generate a first subset indicator 786, specifying which credit unions are included in the first subset.

At step 608, the credit unions in the first subset are caused to be ranked based at least in part on fees that are charged by the credit unions in the first subset. The fees that are charged by a credit union being relatively low corresponds to a relatively high rank of the credit union. The fees that are charged by a credit union being relatively high corresponds to a relatively low rank of the credit unions. In an example implementation, the ranking logic 766 causes the credit unions in the first subset to be ranked based at least in part on fee information 788, which indicates the fees that are charged by the credit unions in the first subset.

At step 610, a second subset of the plurality of credit unions is caused to be selected from the first subset based at least in part on each credit union in the second subset having a rank that is greater than or equal to a threshold rank. In an example implementation, the ranking logic 766 causes the second subset of the credit unions to be selected form the first subset. For example, the ranking logic 766 may select the second subset from the first subset. In another example, the ranking logic 766 may instruct the server to do so. In accordance with this example, the ranking logic 766 may receive an indicator from the server, specifying which of the credit unions from the first subset have been selected for inclusion in the second subset. In accordance with this implementation, the ranking logic 766 may generate a second subset indicator 784, indicating which of the credit unions are selected for inclusion in the second subset.

At step 612, a notice is caused to be presented via a user interface that is included in the point-of-sale terminal. The notice identifies each credit union in the second subset and informs the user that the user is eligible for membership in the respective credit union. In an example implementation, the solicitation logic 784 causes a notification 782 to be presented via the user interface. For instance, the solicitation logic 784 provide instructions to the point-of-sale terminal, instructing the point-of-sale terminal to present the notification 782 via the user interface.

It will be recognized that the restricted-access credit device 700 may not include one or more of the healthcare-related restriction logic 712, the identification logic 704, the comparison logic 762, the solicitation logic 764, and/or the ranking logic 766. Furthermore, the restricted-access credit device 700 may include components in addition to or in lieu of the healthcare-related restriction logic 712, the identification logic 704, the comparison logic 762, the solicitation logic 764, and/or the ranking logic 766.

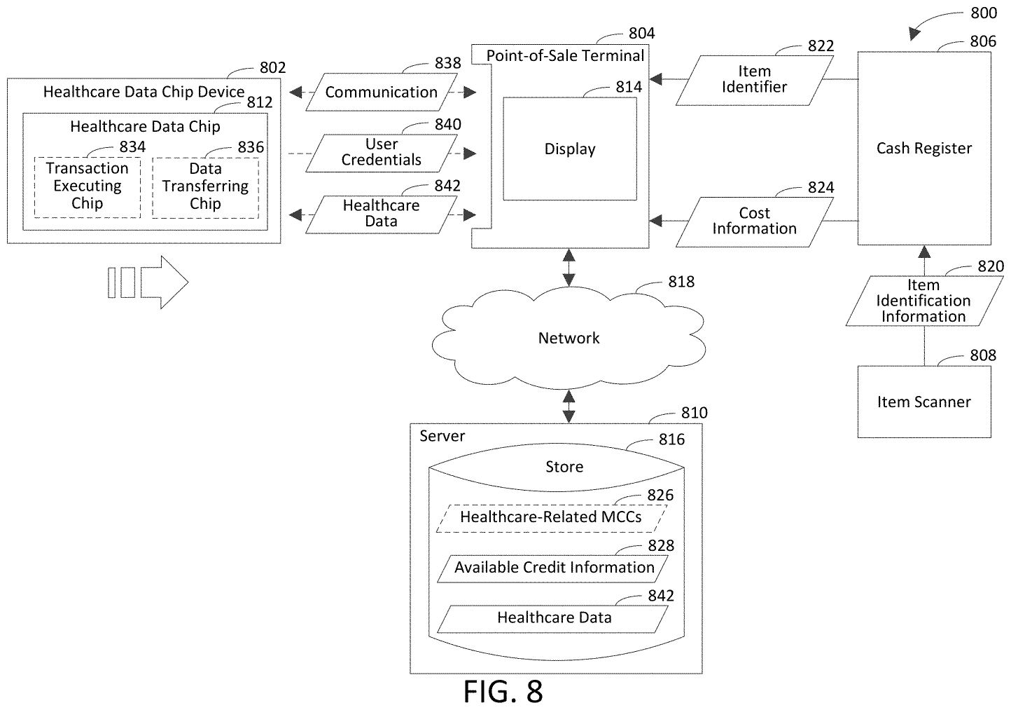

FIG. 8 shows an example healthcare data chip system 800 in accordance with an embodiment. Generally speaking, the healthcare data chip system 800 operates to access healthcare data using a healthcare data chip device 802. As shown in FIG. 8, the healthcare data chip system 800 includes the healthcare data chip device 802, a point-of-sale (POS) terminal 804 (a.k.a. POS reader 804), a cash register 806, an item scanner 808, a server 810, and a network 818. Communication among the POS terminal 804, the cash register 806, the item scanner 808, and the server 810 may be carried out over the network 818 using well-known network communication protocols. The network 818 may be a wide-area network (e.g., the Internet), a local area network (LAN), another type of network, or a combination thereof. The POS terminal 804 is shown in FIG. 8 to be coupled to the server 810 via the network 818 for non-limiting, illustrative purposes. The cash register 806 is shown to be coupled to the POS terminal 804 and the item scanner 808 without going through the network 818 for non-limiting, illustrative purposes to show that communication therebetween need not necessarily pass through the network 818. Communication between the healthcare data chip device 802 and the POS terminal 804 may be carried out over the network 818 and/or using any other suitable technology (e.g., near-field communication or physical contact). Any of the connections between the POS terminal 804, the cash register 806, the item scanner 808, and the server 810 may be wireless or wired.

The healthcare data chip device 802 is a processing system that is capable of communicating with the POS terminal 804. The processing system may be incorporated into any suitable object, including but not limited to a ring, a necklace, an implantable device, etc. For instance, the implantable device may be implanted beneath the skin of the user to whom the line of credit is granted or a pet of the user. It will be recognized that the user may be a human or a non-human animal (e.g., a pet). For example, the healthcare data chip device 802 may be used in a veterinary setting.

The healthcare data chip device 802 may be configured to communicate with the POS terminal 804 in accordance with the UPOS standard. A variety of platform-specific implementations of the UPOS standard exist. One example platform-specific implementation is OPOS. The healthcare data chip device 802 and the POS terminal 804 may communicate in accordance with any such implementation of the UPOS standard.

Communication between the healthcare data chip device 802 and the POS terminal 804 may be initiated in any of a variety of ways. For example, the healthcare data chip device 802 may be physically inserted at least partially into the POS terminal 804. In accordance with this example, circuitry in the healthcare data chip 812 may come into physical contact with conductive leads in the POS terminal 804, thereby establishing a communication channel. For instance, the physical contact may form an electrical contact between the healthcare data chip device 802 and the POS terminal 804. In another example, the healthcare data chip device 802 may be placed within a designated proximity from the POS terminal 804. In accordance with this example, any of a variety of near-field communication protocols may be used to establish a communication channel between the healthcare data chip device 802 and the POS terminal 804. For instance, the communication channel may be established via electrical coupling and/or magnetic coupling.

The healthcare data chip device 802 includes healthcare data chip 812. The healthcare data chip 812 is configured to access healthcare data 842 (e.g., aggregated healthcare data), which is stored at a store 816 in the server 810, via the POS terminal 804 in accordance with any one or more of the techniques described herein. The healthcare data chip 812 establishes communication 838 with the POS terminal 804 on behalf of the user. The communication 838 initiates a purchase of good(s) and/or service(s). For instance, the good(s) may include healthcare-related good(s), and the service(s) may include healthcare-related service(s).

In an example implementation, the healthcare data chip 812 establishes the communication 838 with the POS terminal 804 by receiving a purchase request that requests execution of a purchase transaction for the good(s) and/or the service(s) from the POS terminal 804. In accordance with this implementation, the healthcare data chip 812 establishes the communication 838 with the POS terminal 804 further by providing an account identifier that identifies the line of credit associated with the healthcare data chip device 802 to the POS terminal 804. The account identifier 832 may include a transaction-agnostic account number that identifies the line of credit or a transaction-specific identifier (e.g., in lieu of the transaction-agnostic account number) that identifies the line of credit, as described above with reference to FIG. 1. For instance, the transaction-agnostic account number is configured to be used for multiple (e.g., all) purchase transactions that are executed using the healthcare data chip device 802; whereas, the transaction-specific identifier is configured to be used for a single purchase transaction.

The healthcare data chip 812 authenticates the user to the store 816 by providing credentials of the user (i.e., user credentials) 840 to the store 816. For example, the healthcare data chip 812 may provide the user credentials 840 to the store 816 via the POS terminal 804. In another example, the healthcare data chip 812 may provide the user credentials 840 to the store 816 via another channel (e.g., via a cellular connection) without passing the user credentials 840 through the POS terminal 804.

The healthcare data chip 812 obtains access to the healthcare data 842 via the POS terminal 804 based at least in part on the user credentials 840 matching reference user credentials that are stored at the store 816 or that are otherwise accessible to the store 816. The user credentials 840 may be deemed to match the reference user credentials based on the user credentials 840 and the reference credentials being the same. The healthcare data 842 may include portions that are aggregated from respective healthcare data storage systems. For instance, the healthcare data chip 812 may aggregate the portions to the store 816 by uploading the portions to the store 816. Each of the healthcare data storage systems may not share its portion of the healthcare data 842 with any of the other healthcare data storage systems. In response to the portions of the healthcare data 842 being aggregated to the store 810, the healthcare data chip 812 may download one or more of the portions and distribute those portion(s) to any one or more of the healthcare data storage systems.

It will be recognized that the healthcare data chip 812 may store the portions of the healthcare data 842 in memory of the healthcare data chip 812 in lieu of or in addition to causing the portions to be stored at the store 816. If the portions of the healthcare data 842 are stored in the memory of the healthcare data chip 812, the portions may be temporarily stored in the memory so that the portions can be uploaded to the cloud, and the portions may be deleted from the memory after the portions are uploaded to the cloud. In this manner, the health care data chip device 802 may serve as a conduit through which the portions are transferred from the disparate healthcare data storage systems to the server 810. Alternatively, the portions may remain stored in the memory of the healthcare data chip 812 after the portions are uploaded to the server 810.

In response to downloading a portion of the healthcare data 842 from a healthcare data storage system, the healthcare data chip 812 may automatically provide the portion (e.g., any one or more records therein) to the store 816 for subsequent access. Accordingly, the records may be stored (e.g., persistently stored) in the cloud.

The healthcare data chip 802 may allow the user and/or others who have access to the healthcare data chip device 802 to access the healthcare data 842 under designated circumstances. For example, Baylor Medical Center uses an EHR called MyChart®, which enables the user, once the user is in the MyChart® system, to log in with the user's username and password. In the MyChart® system, the user can access only the user's own healthcare data. One issue encountered by MyChart® and other electronic health record systems is that not every consumer is using that EHR. Still, there are various versions of EHRs and patient portals that people do not use as much. Another issue encountered by MyChart®, My Patient Portal, and other electronic versions of health records and patient portals is that the healthcare information stored thereon is accessible using the Internet. Consumers may be leery of storing their healthcare data on the internet because they may believe that the healthcare data may be hacked or stolen. The healthcare data 842 that is accessed using the healthcare data chip device 802 may be owned by the user; whereas, the healthcare data accessible through an EHR traditionally is owned by the providers. The healthcare data chip device 802 may be capable of placing tools in the consumer's hands, so that the user may take charge of more of the user's healthcare data and decisions. Accordingly, the person making the payment may own the medical records. The healthcare data chip device 802 may be used as a tool to collect, transfer, and discharge healthcare data at the point-of-sale (e.g., with digital payments, credit payments, etc.). The healthcare data chip device 802 may be able to access educational information, informative healthcare information regarding options of the user (e.g., surgical treatment options, non-surgical treatment options, drug treatment options).

The healthcare data chip 812 causes at least some of the healthcare data 842 to be presented via a user interface (e.g., display 814) that is included in the POS terminal 804 based at least in part on access to the healthcare data 842 being obtained. For instance, the healthcare data chip 812 may download a subset of the healthcare data 842 and provide the subset to the display 814 for presentation to the user and/or to others. The healthcare data chip 812 may provide the subset to the display 814 based on (e.g., based at least in part on) the subset being related to or relevant to the purchase that is initiated by the communication 838.

In veterinary embodiments, the user is a non-human animal, or the user is a human who uses the healthcare data chip device 802 for the benefit of a non-human animal. In such embodiments, the healthcare data chip 812 may cause a bloodline database that is included in the store to be traversed. For instance, the bloodline database may include a row for each bloodline. The bloodline database may include multiple columns, and each row may include multiple cells corresponding to the respective columns. At least one cell in each row may indicate at least one illness or infectious disease that has occurred with regard to the corresponding bloodline. The healthcare data chip 812 may cause a bloodline of the non-human animal to be compared to the bloodlines in the bloodline database. For instance, the healthcare data chip 812 may compare the bloodline of the non-human animal to the bloodlines in the bloodline database, or the healthcare data chip 812 may instruct the store 816 to do so. Upon identifying the bloodline of the non-human animal in the bloodline database, illness(es) and/or infectious disease(s) that have occurred with regard to the corresponding bloodline may be identified by reviewing the cells that are included in the row. For instance, the healthcare data chip 812 may review the cells to identify the illness(es) and/or the infectious disease(s), or the healthcare data chip 812 may instruct the store 816 to do so. Moreover, the healthcare data chip 812 may cause the causes of the illness(es) and/or infectious disease(s) that have occurred with regard to the corresponding bloodline to be determined (e.g., by analyzing the healthcare data 842 of the non-human animal).

The healthcare data chip 812 is shown in FIG. 8 to include a transaction executing chip 834 and a data transferring chip 836 for non-limiting illustrative purposes. The transaction executing chip 834 is configured to provide payment for purchases of healthcare-related services and health-related goods. For instance, the transaction executing chip 834 may be configured strictly for this purpose (e.g., for this purpose only). The data transferring chip 836 is configured to provide access to healthcare data 842. The data transferring chip 836 may be configured strictly for this purpose. The data transferring chip 836 (and more broadly, the healthcare data chip 812 and the healthcare data chip device 802) may be agnostic with regard to the various healthcare data storage systems (e.g., electronic medical records software systems). Accordingly, the data transferring chip 836 may be capable of gathering subsets of the healthcare data 842 from any of a variety of electronic medical records software systems (e.g., Allscripts, GE, Cerner, Epic).

In an example, the data transferring chip 836 may download data from a medical record system and/or upload data to the medical record system. In accordance with this example, the uploaded data may pertain to a transaction that is executed by the transaction executing chip 834. The data transferring chip 836 may be configured to transfer (e.g., automatically transfer) medical data from a user's EMR or EHR when the user's healthcare data chip device 802 is inserted into the POS terminal 804. For instance, when the user inserts the healthcare data chip device 802 into the POS terminal 804, the data transferring chip 836 may extract, distribute, and manage the medical data at the same time the transaction executing chip 834 provides payment. It will be recognized that the transaction executing chip 834 and the data transferring chip 836 may be fabricated as separate chips or may be combined into (e.g., fabricated as) a common (e.g., single) chip, such as the healthcare data chip 812, as shown in FIG. 8. Each of the transaction executing chip 834 and the data transferring chip 836 may include one or more processors to perform operations associated with the respective chip. If the transaction executing chip 834 and the data transferring chip 836 are combined into a common chip, the common chip may include one or more processors to perform operations associated with the chips.

The healthcare data chip device 802 may be linked to a restricted-access credit device (e.g., restricted-access credit device 102, 400, or 700) described above with reference to FIGS. 1-7. In an example implementation, the healthcare data chip device 802 may integrate the restricted-access credit device, resulting in one device having both functionalities. In another example implementation, the healthcare data chip device 802 and the restricted-access credit device are distinct articles.

The healthcare data chip device 802 may operate on the infrastructure (a.k.a. rails) of any one or more credit card providers (e.g., Mastercard Incorporated; Visa, Inc.; Discover Financial Services, Inc.; American Express Company). For instance, any entity that accepts credit cards of the provider will accept the healthcare data chip device 802.

The healthcare data chip 812 may be implemented in various ways to access healthcare data 842 via the POS terminal 804 including being implemented in hardware, software, firmware, or any combination thereof. For example, at least a portion of the healthcare data chip 812 may be implemented as computer program code configured to be executed in one or more processors. In another example, at least a portion of the healthcare data chip 812 may be implemented as hardware logic/electrical circuitry. For instance, at least a portion of the healthcare data chip 812 may be implemented in a field-programmable gate array (FPGA), an application-specific integrated circuit (ASIC), an application-specific standard product (ASSP), a system-on-a-chip system (SoC), a complex programmable logic device (CPLD), etc. Each SoC may include an integrated circuit chip that includes one or more of a processor (e.g., a microcontroller, microprocessor, digital signal processor (DSP), etc.), memory, one or more communication interfaces, and/or further circuits and/or embedded firmware to perform its functions.

The item scanner 808 and the cash register 806 are operable in a manner similar to the item scanner 108 and the cash register 106, respectively, described above with reference to FIG. 1. The item identification information 820 provided by the item scanner 808 corresponds to the item identification information 120 described above with reference to FIG. 1. The item identifier 822 and the cost information 824 provided by the cash register 806 correspond to the item identifier 122 and the cost information 124, respectively, described above with reference to FIG. 1.

The POS terminal 804 is a processing system that is capable of communicating with the healthcare data chip device 802. The POS terminal 804 acts as an intermediary that enables the healthcare data chip device 802 to access the healthcare data 842 through the network 818. For instance, the POS terminal 804 may be said to access the healthcare data 842 on behalf of the healthcare data chip device 802 (e.g., based at least in part on instructions that are received from the healthcare data chip device 802). To this end, the POS terminal 804 receives the user credentials 840 from the healthcare data chip device 802 and forwards (e.g., automatically forwards) the user credentials 840 to the store 816 for processing. The POS terminal 804 receives the healthcare data 842 (e.g., at least a subset thereof) in response to the store 816 confirming that the user credentials 840 match the reference credentials that are accessible to the store 816. Upon receipt of the healthcare data 842, the POS terminal 804 forwards the healthcare data 842 to the healthcare data chip device 802. The POS terminal 804 includes the display 814, which is configured to display the healthcare data 842 in response to instructions received from the healthcare data chip device 802.

The POS terminal 804 is also configured to interact with the healthcare data chip device 802 to facilitate (e.g., process) purchases of goods and services. To this end, the POS terminal 804 generates a purchase request, which may include the MCC of the merchant that is associated with the POS terminal 804. The POS terminal 804 may provide an item identifier or information derived therefrom for each item that is to be purchased to the healthcare data chip device 802, though the scope of the example embodiments is not limited in this respect. The display 814 of the POS terminal 804 is configured to display information regarding each item that is to be purchased. For instance, the display 814 may display the item identifier and the corresponding cost information for each item that is to be purchased. The display 814 may further display a cumulative cost for the items that are to be purchased. The POS terminal 804 may generate the purchase request based at least in part on the cumulative cost for the items that are to be purchased. The POS terminal 804 may generate the purchase request further based on the MCC of the merchant that is associated with the POS terminal 804.

The POS terminal 804 receives an account identifier that identifies the line of credit associated with the healthcare data chip device 802 from the healthcare data chip device 802. The POS terminal 804 selectively applies the cumulative cost against the line of credit. In some restricted-access embodiments (i.e., embodiments in which the healthcare data chip device 802 includes functionality of a restricted-access credit device, such as restricted-access credit device 102 or 400), the POS terminal 804 selectively applies the cumulative cost against the line of credit depending on (e.g., based at least in part on) whether the healthcare data chip device 802 triggers execution of the purchase transaction. The POS terminal 804 may selectively apply the cumulative cost against the line of credit further depending on whether the available credit associated with the line of credit is greater than or equal to the cumulative cost for the items. For instance, the POS terminal 804 may access (e.g., retrieve) available credit information 828, which indicates the amount of available credit associated with the line of credit, from the server 810 to determine whether the available credit associated with the line of credit is greater than or equal to the cumulative cost for the items. It will be recognized that the POS terminal 804 may include the cash register 806 and/or the item scanner 808, though the scope of the example embodiments is not limited in this respect.

The server 810 includes the store 816, which stores healthcare-related MCCs 826, the available credit information 828, and the healthcare data 842. The server 810 is configured to provide access to the healthcare-related MCCs 826, the available credit information 828, and the healthcare data 842 in response to requests for access that are received from the POS terminal 804. The store 816 may be a database, such as a relational database, an entity-relationship database, an object database, an object relational database, or an extensible markup language (XML) database.

In veterinary embodiments in which the user is a non-human animal or in which the user is a human who uses the healthcare data chip device 802 for the benefit of a non-human animal, the healthcare data 842 pertains to the non-human animal. For instance, the healthcare data 842 may include information about vaccinations, injuries, treatments, worming, tech filing, blacksmithing, pedigree, and/or breed of the non-human animal. The store 816 may store a bloodline database that cross-references bloodlines with illnesses and infectious diseases that are associated with the bloodlines.

In some restricted-access embodiments, the purchase request that the POS terminal 804 provides to the healthcare data chip device 802 includes a merchant category code (MCC) of a merchant that is associated with the POS terminal 804. In such embodiments, the healthcare data chip 812 causes the MCC of the merchant to be cross-referenced with healthcare-related MCCs (e.g., the healthcare-related MCCs 826). For example, the healthcare data chip 812 may cross-reference the MCC of the merchant with the healthcare-related MCCs 826. In accordance with this example, the healthcare data chip 812 may retrieve the healthcare-related MCCs 826 from the server 810 (e.g., via the POS terminal 804) for comparison with the MCC of the merchant. In another example, the healthcare data chip 812 may instruct the server 810 to cross-reference the MCC of the merchant with the healthcare-related MCCs 826. In accordance with this example, the healthcare data chip 812 may forward the MCC of the merchant to the server 810 (e.g., via the POS terminal 804) along with an instruction, which instructs the server 810 to cross-reference the MCC of the merchant with the healthcare-related MCCs 826. In further accordance with this example, the healthcare data chip 812 may receive an indicator from the server 810 that indicates whether the MCC of the merchant corresponds to at least one of the healthcare-related MCCs 826.

In both of the examples mentioned above, the MCC of the merchant corresponding to at least one of the healthcare-related MCCs 826 may indicate that the purchase transaction is to be executed (e.g., execution is to be triggered). Whereas, the MCC of the merchant not corresponding to at least one of the healthcare-related MCCs 826 may indicate that the purchase transaction is not to be executed (e.g., execution is not to be triggered).

In accordance with these restricted-access embodiments, the healthcare data chip 812 selectively triggers execution of the purchase transaction depending on (e.g., based at least in part on) whether the MCC of the merchant corresponds to at least one of the healthcare-related MCCs. For example, the healthcare data chip 812 may trigger execution of the purchase transaction in response to (e.g., based at least in part on) the MCC of the merchant corresponding to at least one of the healthcare-related MCCs 826 by executing the purchase transaction or by instructing the POS terminal 804 to execute the purchase transaction. In another example, the healthcare data chip 812 may not trigger execution of the purchase transaction in response to (e.g., based at least in part on) the MCC of the merchant not corresponding to at least one of the healthcare-related MCCs 826 by not executing the purchase transaction and by not instructing the POS terminal 804 to execute the purchase transaction.

In accordance with these restricted-access embodiments, the healthcare data chip 812 may selectively trigger execution of the purchase transaction further depending on whether one or more additional criteria are satisfied, as described above with reference to FIG. 1. For instance, the healthcare data chip 812 may selectively trigger execution of the purchase transaction further depending on whether the stock keeping unit (SKU) associated with the item corresponds to at least one pre-approved SKU. For example, a pre-approved SKU may be a SKU that is approved prior to the account identifier being provided to the POS terminal 804 by the healthcare data chip device 802 and prior to the purchase request being received at the healthcare data chip device 802 from the POS terminal 804.

The healthcare data chip 812 may cause the SKU associated with the item to be cross-referenced with pre-approved SKUs. For example, the healthcare data chip 812 may cross-reference the SKU associated with the item with pre-approved SKUs, which may be stored at the store 816 or otherwise accessible to the store 816. In accordance with this example, the healthcare data chip 812 may retrieve the pre-approved SKUs from the server 810 (e.g., via the POS terminal 804) for comparison with the SKU associated with the item. In another example, the healthcare data chip 812 may instruct the server 810 to cross-reference the SKU associated with the item with the pre-approved SKUs. In accordance with this example, the healthcare data chip 812 may forward the SKU of the item to the server 810 (e.g., via the POS terminal 804) along with an instruction, which instructs the server 810 to cross-reference the SKU of the item with the pre-approved SKUs. In further accordance with this example, the healthcare data chip 812 may receive an indicator from the server 810 that indicates whether the SKU of the item corresponds to at least one of the pre-approved SKUs.

In both of the examples mentioned above, the SKU of the item corresponding to at least one of the pre-approved SKUs may indicate that the purchase transaction is to be executed (e.g., execution is to be triggered). Whereas, the SKU of the item not corresponding to at least one of the pre-approved SKUs may indicate that the purchase transaction is not to be executed (e.g., execution is not to be triggered), even if the MCC of the merchant corresponds to at least one of the healthcare-related MCCs 826.

For example, the healthcare data chip 812 may trigger execution of the purchase transaction in response to (e.g., based at least in part on) the SKU of the item corresponding to at least one pre-approved SKU and further in response to the MCC of the merchant corresponding to at least one of the healthcare-related MCCs 826 by executing the purchase transaction or by instructing the POS terminal 804 to execute the purchase transaction. In another example, the healthcare data chip 812 may not trigger execution of the purchase transaction in response to (e.g., based at least in part on) the SKU of the item not corresponding to at least one pre-approved SKU, even if the MCC of the merchant corresponds to at least one of the healthcare-related MCCs 826, by not executing the purchase transaction and by not instructing the POS terminal 804 to execute the purchase transaction.

In accordance with these restricted-access embodiments, the healthcare data chip device 802 and/or the store 816 may store a policy that identifies the healthcare-related MCCs 826. The healthcare data chip 812 may enforce the policy by selectively triggering execution of the purchase transaction depending on whether the MCC of the merchant corresponds to at least one of the healthcare-related MCCs.

It will be recognized that the healthcare data chip system 800 may not include all of the components shown in FIG. 8. Furthermore, the healthcare data chip system 800 may include components in addition to or in lieu of those shown in FIG. 8.

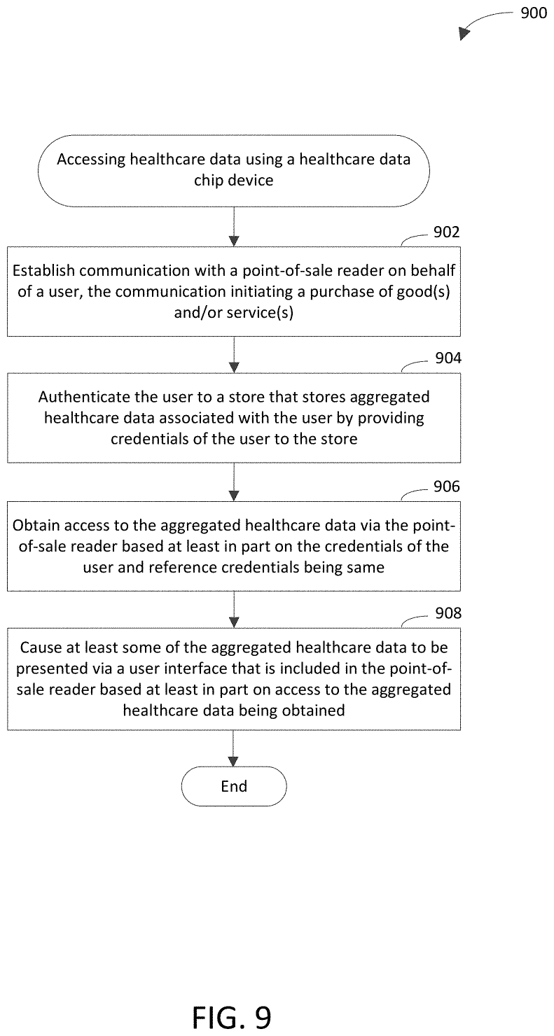

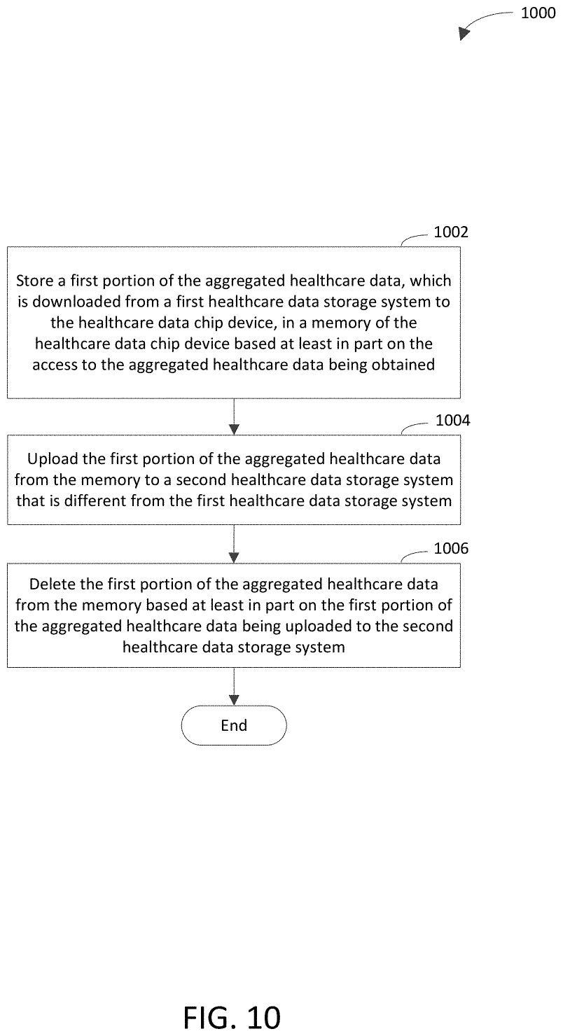

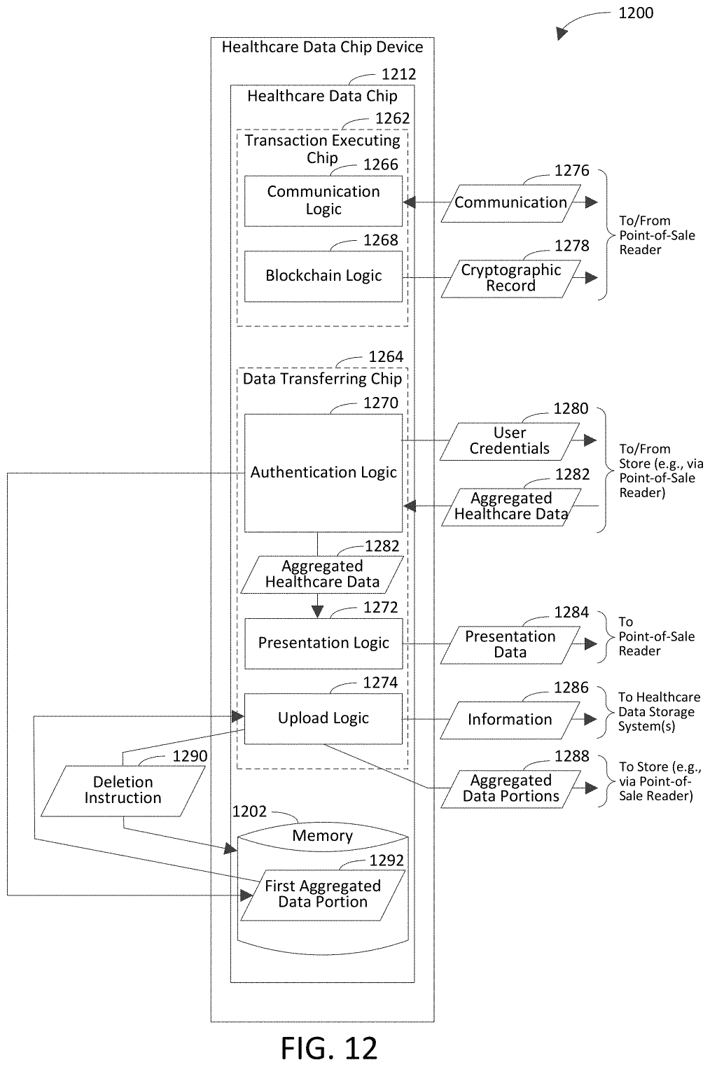

FIGS. 9-10 depict flowcharts 900 and 1000 of example methods for accessing healthcare data using a healthcare data chip device in accordance with embodiments. Flowcharts 900 and 1000 may be performed by the healthcare data chip device 802 shown in FIG. 8, for example. For illustrative purposes, flowcharts 900 and 1000 are described with respect to the healthcare data chip device 1200 shown in FIG. 12, which is an example implementation of the healthcare data chip device 802, according to an embodiment. As shown in FIG. 12, the healthcare data chip device 1200 includes a healthcare data chip 1212. The healthcare data chip 1200 includes a transaction executing chip 1262, a data transferring chip 1264, and memory 1202. The transaction executing chip 1262 includes communication logic 1266 and blockchain logic 1268. The data transferring chip 1264 includes authentication logic 1270, presentation logic 1272, and upload logic 1274. Further structural and operational embodiments will be apparent to persons skilled in the relevant art(s) based on the discussion regarding flowcharts 900 and 1000.

As shown in FIG. 9, the method of flowchart 900 begins at step 902. In step 902, communication is established with a point-of-sale reader on behalf of a user. The communication initiates a purchase of good(s) and/or service(s). In an example implementation, the communication logic 1266 establishes communication 1276 with the point-of-sale reader.

At step 904, the user is authenticated to a store that stores aggregated healthcare data associated with the user by providing credentials of the user to the store. For example, the credentials of the user may be provided to the store via the point-of-sale reader. In another example, the credentials of the user may be provided to the store via another communication path (e.g., via a cellular connection) without the credentials passing through the point-of-sale reader. In accordance with this example, the store may send a request for the credentials to a cell phone of the user when an attempt (e.g., request) to obtain access the aggregated healthcare data is detected. In an example implementation, authentication logic 1270 authenticates the user to the store that stores aggregated healthcare data 1282 associated with the user by providing user credentials 1280 of the user to the store.

At step 906, access to the aggregated healthcare data is obtained via the point-of-sale reader based at least in part on the credentials of the user and reference credentials being same. For instance, access to the aggregated healthcare data may be obtained by downloading the aggregated healthcare data through the point-of-sale reader. The aggregated healthcare data includes portions that are aggregated from respective healthcare data storage systems that do not share the respective portions of the aggregated healthcare data with others of the healthcare data storage systems. For instance, the healthcare data storage systems may not be compatible with each other. Accordingly, the healthcare data storage systems may not be capable of communicating with each other. In a first example, the credentials of the user indicate a DNA profile of the user, and the reference credentials include a reference DNA profile. In a second example, the credentials of the user indicate information regarding blood (e.g., a blood type) of the user, and the reference credentials include information regarding a reference blood sample. In a third example, the credentials of the user indicate information regarding saliva of the user, and the reference credentials include information regarding a reference saliva sample. In a fourth example, the credentials of the user indicate information regarding a fingerprint of the user, and the reference credentials include information regarding a reference fingerprint. In a fifth example, the credentials of the user include information regarding an iris scan of the user, and the reference credentials include information regarding a reference iris scan. In a sixth example, the credentials of the user include information regarding a retinal scan of the user, and the reference credentials include information regarding a reference retinal scan. The reference credentials may be stored in the store or stored externally from the store. In an example implementation, authentication logic 1270 obtains access to the aggregated healthcare data 1282 via the point-of-sale reader based at least in part on the user credentials 1280 and the reference credentials being the same. For instance, the store may grant access to the aggregated healthcare data 1282 based at least in part on a cross-reference of the user credentials 1280 with the reference credentials indicating that the user credentials 1280 and the reference credentials are the same.

At step 908, at least some of the aggregated healthcare data is caused to be presented via a user interface that is included in the point-of-sale reader based at least in part on access to the aggregated healthcare data being obtained. In an example implementation, the presentation logic 1272 causes presentation data 1284 to be presented via the user interface based at least in part on the access to the aggregated healthcare data 1282 being obtained. In accordance with this implementation, the presentation data 1284 includes at least some of the aggregated healthcare data 1282. The authentication logic 1270 may forward the presentation data 1284 to the presentation logic 1272 to enable the presentation logic 1272 to cause the presentation data 1284 to be presented to the user via the user interface. For instance, the presentation logic 1272 may cause the presentation data 1284 to be presented via the user interface by forwarding the presentation data 1284 to the point-of-sale reader (e.g., the user interface therein).

In an example embodiment, establishing the communication at step 902 includes causing the purchase of the good(s) and/or the service(s) to be initiated using a first processor that is included in a first chip by establishing the communication with the point-of-sale reader. In accordance with this embodiment, obtaining the access to the aggregated healthcare data at step 906 includes obtaining the access to the aggregated healthcare data associated with the user using a second processor that is included in a second chip that is different from the first chip. For instance, the second chip may be external to and/or separate from the first chip.

In another example embodiment, establishing the communication with the point-of-sale reader at step 902 and obtaining the access to the aggregated healthcare data associated with the user at step 906 are performed using a common computer chip.

In yet another example embodiment, the good(s) and/or the service(s) include a medication. In accordance with this embodiment, the at least some of the aggregated healthcare data includes information specifying a potential consequence of mixing the medication with one or more other substances (e.g., based at least in part on a statistical likelihood of the potential consequence occurring being greater than or equal to a threshold likelihood). Examples of such a substance include but are not limited to a medication, a food, or a beverage (e.g., an alcoholic beverage, such as beer or wine).

In still another example embodiment, the good(s) and/or the service(s) include a first medication and a second medication. In accordance with this embodiment, the at least some of the aggregated healthcare data includes information specifying a potential consequence of mixing the first medication and the second medication based at least in part on the first medication and the second medication being purchased in a common (e.g., same) transaction.

In yet another example embodiment, the good(s) and/or the service(s) include healthcare-related good(s) and/or healthcare-related service(s). In accordance with this embodiment, the at least some of the aggregated healthcare data includes information specifying an authorized amount that a provider is authorized to charge for the healthcare-related good(s) and/or the healthcare-related service(s) based at least in part on the purchase of the healthcare-related good(s) and/or the healthcare-related service(s) being initiated. For example, the authorized amount may be an amount that is authorized by a government program, such as Medicare or Medicaid. In another example, the authorized amount may be based on a demographic categorization of the user. A government program may define categories that correspond to respective demographics (e.g., age ranges) of a population. For instance, a first category may correspond to a first demographic that corresponds to an age range between 10 and 20 years of age. A second category may correspond to a second demographic that correspond to an age range between 20 and 30 years of age, and so on. The user may be associated with a category based on the demographic to which the user belongs. For instance, if the user is 19 years old, the user may be categorized into the first category mentioned above because the age of the user is between 10 and 20 years.

In still another example embodiment, the user is a non-human animal. In accordance with this embodiment, the communication initiates the purchase of the good(s) and/or the service(s) for the non-human animal. In further accordance with this embodiment, the healthcare data chip device (e.g., healthcare data chip device 1200) that performs the method of flowchart 900 is implanted under skin of the non-human animal.

In some example embodiments, one or more steps 902, 904, 906, and/or 908 of flowchart 900 may not be performed. Moreover, steps in addition to or in lieu of steps 902, 904, 906, and/or 908 may be performed. For instance, in an example embodiment, the method of flowchart 900 further includes uploading information regarding the good(s) and/or the service(s) to each of the healthcare data storage systems through the point-of-sale reader. In an example implementation, the upload logic 1274 uploads information 1286 regarding the good(s) and/or the service(s) to each of the healthcare data storage systems through the point-of-sale reader.

In another example embodiment, the good(s) and/or the service(s) include healthcare-related good(s) and/or healthcare-related service(s). In accordance with this embodiment, the method of flowchart 900 further includes aggregating the portions of the aggregated healthcare data (a.k.a. aggregated data portions) from the respective healthcare data storage systems by uploading the portions to the store. For instance, the upload logic 1274 may aggregate aggregated data portions 1288 from the respective healthcare data storage systems by uploading the aggregated data portions 1288 to the store. In further accordance with this embodiment, uploading the portions to the store includes uploading information relating to the purchase of the healthcare-related good(s) and/or the healthcare-related service(s) via the point-of-sale reader to the store. In further accordance with this embodiment, the at least some of the aggregated healthcare data is different from (e.g., does not include) the information relating to the purchase of the healthcare-related good(s) and/or the healthcare-related service(s).

In yet another example embodiment, the method of flowchart 900 further includes generating a cryptographic record of purchases, which are initiated via respective communications that are established with one or more point-of-sale readers and which include the purchase that is initiated by the communication with the point-of-sale reader, using blockchain technology. The cryptographic record of the purchases is based on an order of the purchases. In an example implementation, the blockchain logic 1268 generates a cryptographic record 1278 of the purchases. In accordance with this implementation, the purchases include the purchase that is initiated by the communication 1276.

In still another example embodiment, establishing the communication at step 902, authenticating the user at step 904, obtaining the access to the aggregated healthcare data at step 906, and causing the at least some of the aggregated healthcare data to be presented via the user interface at step 908 are performed by the healthcare data chip device. In accordance with this embodiment, the healthcare data chip device is associated with a restricted-access credit device that is external to the healthcare data chip device. In further accordance with this embodiment, the method of flowchart 900 further includes causing, by the healthcare data chip device, a portion of a cost of the good(s) and/or the service(s) that is not covered by a healthcare plan (e.g., of the user) to be applied against a line of credit associated with the restricted-access credit device. For instance, the portion of the cost may be applied against the line of credit by being charged to the line of credit. In an aspect of this embodiment, the healthcare plan may be a Medicare plan or a Medicaid plan (e.g., a Part B Medicare plan). In an example implementation, communication logic 1266 causes the portion of the cost of the good(s) and/or the service(s) that is not covered by the healthcare plan to be applied against the line of credit. It will be recognized that the restricted-access credit device and the healthcare data chip device may be a unitary device.

In another example embodiment, the user is non-human animal. In accordance with this embodiment, the method of flowchart 900 further includes causing a bloodline of the non-human animal to be compared to a plurality of bloodlines that are identified in a bloodline database that is included in the store to identify the bloodline of the non-human animal in the bloodline database. For instance, the authentication logic 1270 may cause the bloodline of the non-human animal to be compared to the plurality of bloodlines. In an example implementation, the authentication logic 1270 compares the bloodline of the non-human animal to the plurality of bloodlines. In another example implementation, the authentication logic 1270 instructs the store to compare the bloodline of the non-human animal to the plurality of bloodlines. In further accordance with this embodiment, the method of flowchart 900 further includes causing illness(es) and/or infectious disease(s) that occur with regard to the bloodline of the non-human animal to be identified based at least in part on the illness(es) and/or the infectious disease(s) being cross-referenced with the bloodline of the non-human animal in the bloodline database. For instance, the authentication logic 1270 may cause the illness(es) and/or infectious disease(s) that occur with regard to the bloodline of the non-human animal to be identified. In an example implementation, the authentication logic 1270 identifies the illness(es) and/or infectious disease(s). In another example implementation, the authentication logic 1270 instructs the store to identify the illness(es) and/or infectious disease(s). In further accordance with this embodiment, causing the at least some of the aggregated healthcare data to be presented via the user interface at step 908 includes causing an identity of the illness(es) or the infectious disease(s) to be presented via the user interface that is included in the point-of-sale reader.

In yet another example embodiment, the method of flowchart 900 may include one or more of the steps shown in flowchart 1000 of FIG. 10. As shown in FIG. 10, the method of flowchart 1000 begins at step 1002. In step 1002, a first portion of the aggregated healthcare data, which is downloaded from a first healthcare data storage system to the healthcare data chip device, is stored in a memory of the healthcare data chip device based at least in part on the access to the aggregated healthcare data being obtained. In an example implementation, the authentication logic 1270 stores a first aggregated data portion 1292, which is a first portion of the aggregated healthcare data 1282, in the memory 1202 based at least in part on the access to the aggregated healthcare data 1282 being obtained. The first aggregated data portion 1292 is downloaded from the first healthcare data storage system to the healthcare data chip device 1200 (e.g., the authentication logic 1270 therein).

At step 1004, the first portion of the aggregated healthcare data is uploaded from the memory to a second healthcare data storage system that is different from the first healthcare data storage system. In an example implementation, the upload logic 1274 uploads the first aggregated data portion 1292 from the memory 1202 to the second healthcare data storage system. For example, the upload logic 1274 may retrieve the first aggregated data portion 1292 from the memory 1202 and then forward the first aggregated data portion 1292 to the second healthcare data storage system. In another example, the first aggregated data portion 1292 may be included in the information 1286 that is provided by the upload logic 1274.

At step 1006, the first portion of the aggregated healthcare data is deleted from the memory based at least in part on the first portion of the aggregated healthcare data being uploaded to the second healthcare data storage system. In an example implementation, the upload logic 1274 deletes the first aggregated data portion 1292 from the memory 1202 based at least in part on the first aggregated data portion 1292 being uploaded to the second healthcare data storage system.

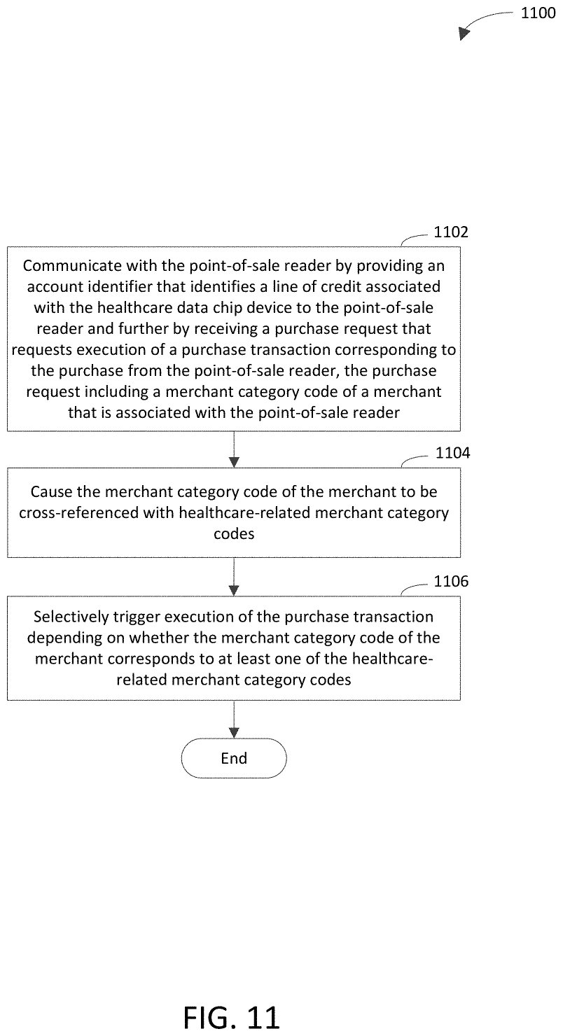

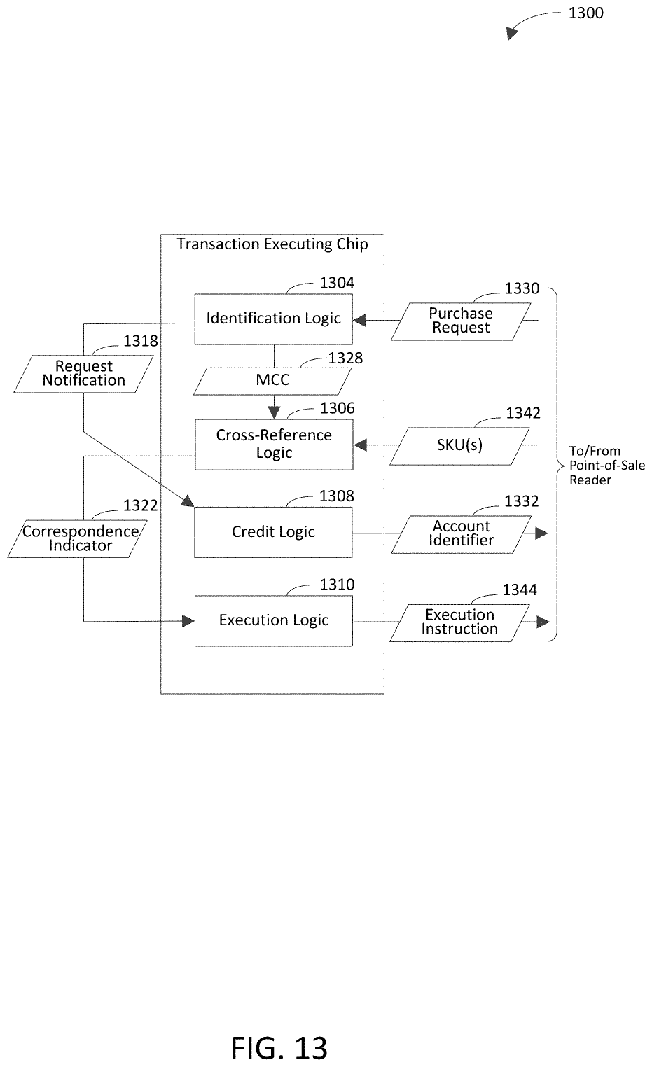

In still another example embodiment, the method of flowchart 900 may include one or more of the steps shown in flowchart 1100 of FIG. 11. FIG. 11 depicts a flowchart 1100 of another example method for accessing healthcare data using a healthcare data chip device in accordance with an embodiment. Flowchart 1100 may be performed by the transaction executing chip 834 or 1262 shown in respective FIG. 8 or 12, for example. For illustrative purposes, flowchart 1100 is described with respect to the transaction executing chip 1300 shown in FIG. 13, which is an example implementation of the transaction executing chip 834 or 1262, according to an embodiment. As shown in FIG. 13, the transaction executing chip 1300 includes identification logic 1304, cross-reference logic 1306, credit logic 1308, and execution logic 1310. Further structural and operational embodiments will be apparent to persons skilled in the relevant art(s) based on the discussion regarding flowchart 1100.

As shown in FIG. 11, the method of flowchart 1100 begins at step 1102. In step 1102, communication with the point-of-sale reader is performed by providing an account identifier that identifies a line of credit associated with the healthcare data chip device to the point-of-sale reader and further by receiving a purchase request that requests execution of a purchase transaction corresponding to the purchase from the point-of-sale reader. The purchase request includes a merchant category code of a merchant that is associated with the point-of-sale reader. In an example implementation, the identification logic 1304 and the credit logic 1308 perform the communication with the point-of-sale reader. In accordance with this implementation, the identification logic 1304 receives a purchase request 1330 that requests execution of a purchase transaction corresponding to the purchase from the point-of-sale reader. The purchase request 1330 includes a merchant category code 1328 of the merchant that is associated with the point-of-sale. The identification logic 1304 identifies the merchant category code 1328 in the purchase request 1330. The identification logic 1304 provides the merchant category code 1328 to the cross-reference logic 1306 for processing. The identification logic 1304 may generate a request notification 1318 to indicate that the purchase request 1330 has been received. In further accordance with this implementation, the credit logic 1308 provides an account identifier 1332 that identifies a line of credit associated with the healthcare data chip device 1200 to the point-of-sale reader. For instance, the credit logic 1308 may provide the account identifier 1332 based at least in part on receipt of the request notification 1318 (e.g., based at least in part on the request notification 1318 indicating that the purchase request 1330 has been received).

At step 1104, the merchant category code of the merchant is caused to be cross-referenced with healthcare-related merchant category codes. In an example implementation, the cross-reference logic 1306 cross-references the merchant category code 1328 with the healthcare-related merchant category codes (e.g., healthcare-related MCCs 826). The cross-reference logic 1306 generates a correspondence indicator 1322 to indicate whether the merchant category code 1328 corresponds to at least one of the healthcare-related merchant category codes.

At step 1106, execution of the purchase transaction is selectively triggered depending on whether the merchant category code of the merchant corresponds to (e.g., matches) at least one of the healthcare-related merchant category codes. In an example implementation, the execution logic 1310 selectively triggers execution of the purchase transaction depending on whether the merchant category code 1328 of the merchant corresponds to at least one of the healthcare-related merchant category codes (e.g., depending on whether the correspondence indicator 1322 indicates that the merchant category code 1328 corresponds to at least one of the healthcare-related merchant category codes. For example, the execution logic 1310 may trigger execution of the purchase transaction based at least in part on the merchant category code 1328 corresponding to (e.g., being the same as) at least one of the healthcare-related merchant category codes. In accordance with this example, the execution logic 1310 may trigger the execution of the purchase transaction by generating an execution instruction 1344 that instructs the point-of-sale to execute the purchase transaction. In another example, the execution logic 1310 may not trigger execution of the purchase transaction based at least in part on the merchant category code 1328 not corresponding to (e.g., not being the same as) at least one of the healthcare-related merchant category codes. In accordance with this example, the execution logic 1310 may not trigger the execution of the purchase transaction by not generating the execution instruction 1344.

In an example embodiment, the method of flowchart 1100 further includes receiving SKU(s) associated with the good(s) and/or service(s). For instance, the cross-reference logic 1306 may receive SKU(s) 1342 of the good(s) and/or service(s). In accordance with this embodiment, the method of flowchart 1100 further includes causing the SKU(s) to be cross-referenced with pre-approved SKU(s) to determine whether each of the SKU(s) corresponds to at least one of the pre-approved SKU(s). For example, each pre-approved SKU may have been pre-approved because it is a healthcare-related SKU. In accordance with this example, SKUs that are healthcare-related may be pre-approved; whereas, SKUs that are not healthcare-related may not be pre-approved. For instance, the cross-reference logic 1306 may cause each of the SKU(s) 1342 to be cross-referenced with the pre-approved SKU(s) to determine whether each of the SKU(s) 1342 corresponds to at least one of the pre-approved SKU(s). For example, the cross-reference logic 1306 may cross-reference each of the SKU(s) 1342 with the pre-approved SKU(s) to make the determination. In another example, the cross-reference logic 1306 may generate an instruction that instructs the point-of-sale reader or a server with which the cross-reference reference logic 1306 communicates (via the point-of-sale reader) to cross-reference each of the SKU(s) 1342 with the pre-approved SKU(s). In accordance with this example, the cross-reference logic 1306 may make the determination based on an indicator that is received from the point-of-sale reader or the server and that indicates whether each of the SKU(s) 1342 corresponds to at least one of the pre-approved SKU(s). For instance, the indicator may indicate which of the SKU(s) 1342 correspond to at least one of the pre-approved SKU(s).

In further accordance with this embodiment, selectively triggering the purchase transaction at step 1106 includes selectively including each of the good(s) and/or service(s) in the purchase transaction depending on whether the corresponding SKU of the respective good or service corresponds to (e.g., matches) at least one of the pre-approved SKU(s). For instance, the execution logic 1310 selectively includes each of the good(s) and/or service(s) in the purchase transaction depending on whether the corresponding SKU of the respective good or service corresponds to at least one of the pre-approved SKU(s). For example, the execution logic 1310 may include a good or service in the purchase transaction based at least in part on the SKU of the good or service corresponding to at least one of the pre-approved SKU(s). In another example, the execution logic 1310 may not include a good or service in the purchase transaction based at least in part on the SKU of the good or service not corresponding to at least one of the pre-approved SKU(s).

It will be recognized that the restricted-access credit devices described herein (e.g., restricted-access credit device 102, restricted-access credit device 400, or restricted-access credit device 700) may include any of the functionality of the healthcare data chip devices described herein (e.g., healthcare data chip device 802 or healthcare data chip device 1200). Accordingly, the restricted-access credit devices described herein may include any of the healthcare data chip 812, the transaction executing chip 834, and/or the data transferring chip 836 of FIG. 8; the healthcare data chip 1212, the transaction executing chip 1262, the data transferring chip 1264, the memory 1202, the communication logic 1266, the blockchain logic 1268, the authentication logic 1270, the presentation logic 1272, and/or the upload logic 1274 of FIG. 12; and/or the transaction executing chip 1300, the identification logic 1304, the cross-reference logic 1306, the credit logic 1308, and/or the execution logic 1310 of FIG. 13. The restricted-access credit devices described herein may perform any of the operations described herein with regard to FIGS. 8-13.

Moreover, the healthcare data chip devices described herein may include any of the functionality of the restricted-access credit devices described herein. Accordingly, the healthcare data chip devices described herein may include any of the healthcare-related restriction logic 112 of FIG. 1; the healthcare-related restriction logic 412, the store 402, the identification logic 404, the cross-reference logic 406, the credit logic 408, the execution logic 410, the rating logic 414, and/or the information logic 416 of FIG. 4; and/or the healthcare-related restriction logic 712, the identification logic 704, the comparison logic 762, the solicitation logic 764, and/or the ranking logic 766 of FIG. 7. The healthcare data chip devices described herein may perform any of the operations described herein with regard to FIGS. 1-7.

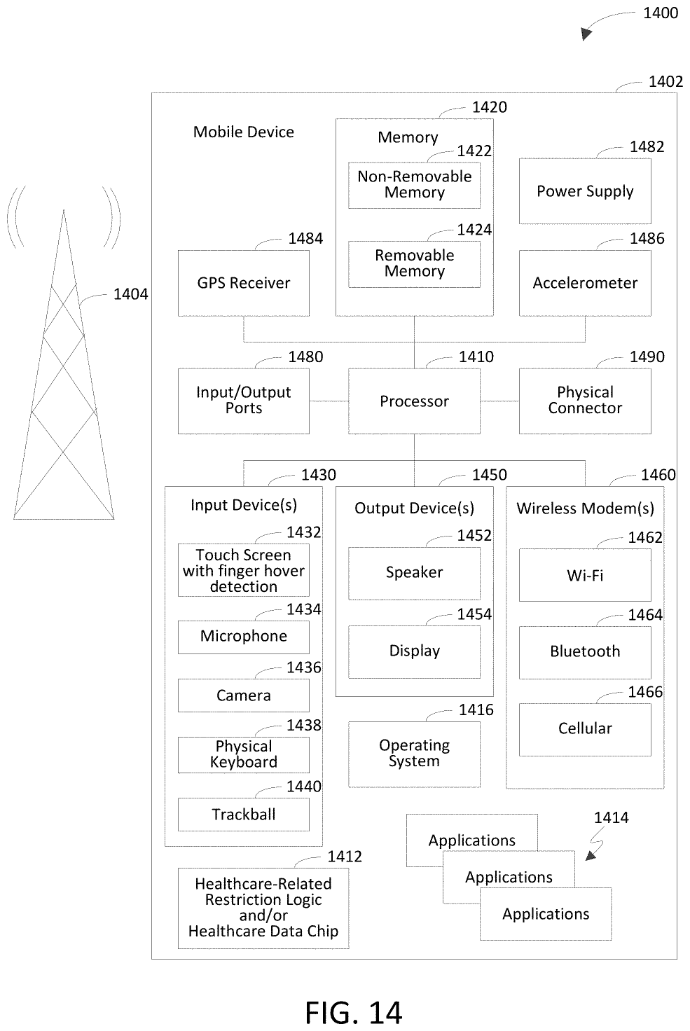

FIG. 14 is a system diagram of an example mobile device 1400 including a variety of optional hardware and software components, shown generally as 1402. Any components 1402 in the mobile device may communicate with any other component, though not all connections are shown, for ease of illustration. The mobile device 1400 may be any of a variety of computing devices (e.g., cell phone, smartphone, handheld computer, Personal Digital Assistant (PDA), etc.) and may allow wireless two-way communications with one or more mobile communications networks 1404, such as a cellular or satellite network, or with a local area or wide area network.

The mobile device 1400 may include a processor 1410 (e.g., signal processor, microprocessor, ASIC, or other control and processing logic circuitry) for performing such tasks as signal coding, data processing, input/output processing, power control, and/or other functions. An operating system 1416 may control the allocation and usage of the components 1402 and support for one or more applications 1414 (a.k.a. application programs). The applications 1414 may include common mobile computing applications (e.g., email applications, calendars, contact managers, web browsers, messaging applications) and any other computing applications (e.g., word processing applications, mapping applications, media player applications).

The mobile device 1400 may include memory 1420. The memory 1420 may include non-removable memory 1422 and/or removable memory 1424. The non-removable memory 1422 may include RAM, ROM, flash memory, a hard disk, or other well-known memory storage technologies. The removable memory 1424 may include flash memory or a Subscriber Identity Module (SIM) card, which is well known in GSM communication systems, or other well-known memory storage technologies, such as “smart cards.” The memory 1420 may store data and/or code for running the operating system 1416 and the applications 1414. Example data may include web pages, text, images, sound files, video data, or other data sets to be sent to and/or received from one or more network servers or other devices via one or more wired or wireless networks. Memory 1420 may store a subscriber identifier, such as an International Mobile Subscriber Identity (IMSI), and an equipment identifier, such as an International Mobile Equipment Identifier (IMEI). Such identifiers may be transmitted to a network server to identify users and equipment.

The mobile device 1400 may support one or more input devices 1430, such as a touch screen 1432, microphone 1434, camera 1436, physical keyboard 1438 and/or trackball 540 and one or more output devices 1450, such as a speaker 1452 and a display 1454. Touch screens, such as the touch screen 1432, may detect input in different ways. For example, capacitive touch screens detect touch input when an object (e.g., a fingertip) distorts or interrupts an electrical current running across the surface. As another example, touch screens may use optical sensors to detect touch input when beams from the optical sensors are interrupted. Physical contact with the surface of the screen is not necessary for input to be detected by some touch screens. For example, the touch screen 1432 may support a finger hover detection using capacitive sensing, as is well understood in the art. Other detection techniques may be used, including but not limited to camera-based detection and ultrasonic-based detection. To implement a finger hover, a user's finger is typically within a predetermined spaced distance above the touch screen, such as between 0.1 to 0.25 inches, or between 0.0.25 inches and 0.05 inches, or between 0.0.5 inches and 0.75 inches, or between 0.75 inches and 1 inch, or between 1 inch and 1.5 inches, etc.

The mobile device 1400 may include healthcare-related restriction logic and/or healthcare data chip 1412. The healthcare-related restriction logic and/or healthcare data chip 1412 is configured to restrict access to a line of credit associated with the mobile device 1400 and/or to access healthcare data in accordance with any one or more of the techniques described herein.

Other possible output devices (not shown) may include piezoelectric or other haptic output devices. Some devices may serve more than one input/output function. For example, touch screen 1432 and display 1454 may be combined in a single input/output device. The input devices 1430 may include a Natural User Interface (NUI). An NUI is any interface technology that enables a user to interact with a device in a “natural” manner, free from artificial constraints imposed by input devices such as mice, keyboards, remote controls, and the like. Examples of NUI methods include those relying on speech recognition, touch and stylus recognition, gesture recognition both on screen and adjacent to the screen, air gestures, head and eye tracking, voice and speech, vision, touch, gestures, and machine intelligence. Other examples of a NUI include motion gesture detection using accelerometers/gyroscopes, facial recognition, 3D displays, head, eye, and gaze tracking, immersive augmented reality and virtual reality systems, all of which provide a more natural interface, as well as technologies for sensing brain activity using electric field sensing electrodes (EEG and related methods). Thus, in one specific example, the operating system 1416 or applications 1414 may include speech-recognition software as part of a voice control interface that allows a user to operate the mobile device 1400 via voice commands. Furthermore, the mobile device 1400 may include input devices and software that allows for user interaction via a user's spatial gestures, such as detecting and interpreting gestures to provide input to a gaming application.

Wireless modem(s) 1460 may be coupled to antenna(s) (not shown) and may support two-way communications between the processor 1410 and external devices, as is well understood in the art. The modem(s) 1460 are shown generically and may include a cellular modem 1466 for communicating with the mobile communication network 1404 and/or other radio-based modems (e.g., Bluetooth 1464 and/or Wi-Fi 1462). At least one of the wireless modem(s) 1460 is typically configured for communication with one or more cellular networks, such as a GSM network for data and voice communications within a single cellular network, between cellular networks, or between the mobile device and a public switched telephone network (PSTN).

The mobile device may further include at least one input/output port 1480, a power supply 1482, a satellite navigation system receiver 1484, such as a Global Positioning System (GPS) receiver, an accelerometer 1486, and/or a physical connector 1490, which may be a USB port, IEEE 1394 (FireWire) port, and/or RS-232 port. The illustrated components 1402 are not required or all-inclusive, as any components may be deleted and other components may be added as would be recognized by one skilled in the art.

Although the operations of some of the disclosed methods are described in a particular, sequential order for convenient presentation, it should be understood that this manner of description encompasses rearrangement, unless a particular ordering is required by specific language set forth herein. For example, operations described sequentially may in some cases be rearranged or performed concurrently. Moreover, for the sake of simplicity, the attached figures may not show the various ways in which the disclosed methods may be used in conjunction with other methods.

Any one or more of the healthcare-related restriction logic 112, the identification logic 404, the cross-reference logic 406, the credit logic 408, the execution logic 410, the healthcare-related restriction logic 412, the rating logic 414, the information logic 416, the identification logic 704, the healthcare-related restriction logic 712, the comparison logic 762, the solicitation logic 764, the ranking logic 766, the communication logic 1266, the blockchain logic 1268, the authentication logic 1270, the presentation logic 1272, the upload logic 1274, the identification logic 1304, the cross-reference logic 1306, the credit logic 1308, the execution logic 1310, flowchart 200, flowchart 300, flowchart 500, flowchart 600, flowchart 900, flowchart 1000, and/or flowchart 1100 may be implemented in hardware, software, firmware, or any combination thereof.

For example, any one or more of the healthcare-related restriction logic 112, the identification logic 404, the cross-reference logic 406, the credit logic 408, the execution logic 410, the healthcare-related restriction logic 412, the rating logic 414, the information logic 416, the identification logic 704, the healthcare-related restriction logic 712, the comparison logic 762, the solicitation logic 764, the ranking logic 766, the communication logic 1266, the blockchain logic 1268, the authentication logic 1270, the presentation logic 1272, the upload logic 1274, the identification logic 1304, the cross-reference logic 1306, the credit logic 1308, the execution logic 1310, flowchart 200, flowchart 300, flowchart 500, flowchart 600, flowchart 900, flowchart 1000, and/or flowchart 1100 may be implemented, at least in part, as computer program code configured to be executed in one or more processors.

In another example, any one or more of the healthcare-related restriction logic 112, the identification logic 404, the cross-reference logic 406, the credit logic 408, the execution logic 410, the healthcare-related restriction logic 412, the rating logic 414, the information logic 416, the identification logic 704, the healthcare-related restriction logic 712, the comparison logic 762, the solicitation logic 764, the ranking logic 766, the healthcare data chip 812, the transaction executing chip 834, the data transferring chip 836, the healthcare data chip 1212, the transaction executing chip 1262, the data transferring chip 1264, the communication logic 1266, the blockchain logic 1268, the authentication logic 1270, the presentation logic 1272, the upload logic 1274, the transaction executing chip 1300, the identification logic 1304, the cross-reference logic 1306, the credit logic 1308, the execution logic 1310, flowchart 200, flowchart 300, flowchart 500, flowchart 600, flowchart 900, flowchart 1000, and/or flowchart 1100 may be implemented, at least in part, as hardware logic/electrical circuitry. Such hardware logic/electrical circuitry may include one or more hardware logic components. Examples of a hardware logic component include but are not limited to a field-programmable gate array (FPGA), an application-specific integrated circuit (ASIC), an application-specific standard product (ASSP), a system-on-a-chip system (SoC), a complex programmable logic device (CPLD), etc. For instance, a SoC may include an integrated circuit chip that includes one or more of a processor (e.g., a microcontroller, microprocessor, digital signal processor (DSP), etc.), memory, one or more communication interfaces, and/or further circuits and/or embedded firmware to perform its functions.

### III. Further Discussion of Some Example Embodiments

An example restricted-access credit device comprises memory and one or more processors coupled to the memory. The one or more processors are configured to communicate with a point-of-sale terminal by providing an account identifier that identifies a line of credit associated with the restricted-access credit device to the point-of-sale terminal and further by receiving a purchase request that requests execution of a purchase transaction for an item from the point-of-sale terminal. The purchase request includes a merchant category code of a merchant that is associated with the point-of-sale terminal. The item includes at least one of one or more goods or one or more services. The one or more processors are further configured to cause the merchant category code of the merchant to be cross-referenced with a plurality of healthcare-related merchant category codes. The one or more processors are further configured to selectively trigger execution of the purchase transaction depending on whether the merchant category code of the merchant corresponds to at least one of the healthcare-related merchant category codes.

In a first aspect of the example restricted-access credit device, the one or more processors are configured to communicate with the point-of-sale terminal further by receiving a stock keeping unit (SKU) associated with the item from the point-of-sale terminal. The SKU is configured to distinguish an item type of the item from other item types of other items. In accordance with the first aspect, the one or more processors are configured to selectively trigger execution of the purchase transaction further depending on whether the SKU associated with the item corresponds to at least one of a plurality of pre-approved stock keeping units.

In a second aspect of the example restricted-access credit device, the one or more processors are configured to communicate with the point-of-sale terminal further by receiving a stock keeping unit (SKU) associated with the item from the point-of-sale terminal. The SKU configured to distinguish an item type of the item from other item types of other items. In accordance with the second aspect, the one or more processors are configured to selectively trigger execution of the purchase transaction further depending on whether the SKU is associated with a healthcare-related item. The second aspect of the example restricted-access credit device may be implemented in combination with the first aspect of the example restricted-access credit device, though the example embodiments are not limited in this respect.

In a third aspect of the example restricted-access credit device, the one or more processors are configured to trigger execution of the purchase transaction by causing a cost of the item to be applied against the line of credit based at least in part on the merchant category code of the merchant corresponding to at least one of the healthcare-related merchant category codes. The third aspect of the example restricted-access credit device may be implemented in combination with the first and/or second aspect of the example restricted-access credit device, though the example embodiments are not limited in this respect.

In a first example of the third aspect, the one or more processors are configured to store information regarding an incentive offered by an employer of a user to whom the line of credit is granted. The incentive offers a credit based at least in part on a purchase of the item from the merchant that is associated with the point-of-sale terminal rather than from another merchant that offers the item for sale. In accordance with the first example of the third aspect, the one or more processors are configured to apply the credit to the line of credit based at least in part on the user purchasing the item from the merchant rather than from another merchant that offers the item for sale.

In a second example of the third aspect, the one or more processors are configured to store information regarding an incentive offered by an employer of a user to whom the line of credit is granted, the incentive offering a credit based at least in part on a purchase of the item from the merchant that is associated with the point-of-sale terminal rather than from another merchant that offers the item for sale. In accordance with the second example of the third aspect, the one or more processors are configured to apply the credit toward an insurance deductible associated with a health insurance policy of the user based at least in part on the user purchasing the item from the merchant rather than from another merchant that offers the item for sale.

In a fourth aspect of the example restricted-access credit device, the restricted-access credit device is linked to a tax-advantaged health benefit account of a user to whom the line of credit is granted. The tax-advantaged health benefit account has funds available for purchases. In accordance with the fourth aspect, the one or more processors are configured to apply the funds of the tax-advantaged health benefit account toward a cost of the item and to apply a difference between the cost and the funds against the line of credit. The fourth aspect of the example restricted-access credit device may be implemented in combination with the first, second, and/or third aspect of the example restricted-access credit device, though the example embodiments are not limited in this respect.

In a fifth aspect of the example restricted-access credit device, the one or more processors are configured to download a transaction history, identifying items purchased with the restricted-access credit device, from a memory that is remote from the restricted-access credit device to the memory that is included in the restricted-access credit device. The fifth aspect of the example restricted-access credit device may be implemented in combination with the first, second, third, and/or fourth aspect of the example restricted-access credit device, though the example embodiments are not limited in this respect.

In an example of the fifth aspect, the one or more processors are configured to prepare an annual tax statement of a user to whom the line of credit is granted based at least in part on the transaction history.

In a sixth aspect of the example restricted-access credit device, the one or more processors are configured to store a policy that identifies the plurality of healthcare-related merchant category codes in the memory. In accordance with the sixth aspect, the one or more processors are configured to enforce the policy by selectively triggering execution of the purchase transaction depending on whether the merchant category code of the merchant corresponds to at least one of the healthcare-related merchant category codes. The sixth aspect of the example restricted-access credit device may be implemented in combination with the first, second, third, fourth, and/or fifth aspect of the example restricted-access credit device, though the example embodiments are not limited in this respect.

In a seventh aspect of the example restricted-access credit device, the one or more processors are configured to assign a plurality of relative priorities to a plurality of respective types of healthcare-related items. In accordance with the seventh aspect, the one or more processors are configured to receive a request for additional credit to be added to the line of credit associated with the restricted-access credit device to cover cost of a medical expense that exceeds an amount of credit available through the line of credit. In further accordance with the seventh aspect, the one or more processors are configured to determine whether the additional credit is to be added to the line of credit by causing information regarding a user to whom the line of credit is granted to be analyzed to determine a creditworthiness rating of the user and further by analyzing information regarding an item with which the medical expense is associated to determine the type of the item with which the medical expense is associated. In further accordance with the seventh aspect, the one or more processors are configured to add the additional credit to the line of credit to cover the cost of the medical expense based at least in part on the creditworthiness rating of the user being greater than or equal to a creditworthiness threshold and further based at least in part on the relative priority of the type of the item with which the medical expense is associated being greater than or equal to a priority threshold. The seventh aspect of the example restricted-access credit device may be implemented in combination with the first, second, third, fourth, fifth, and/or sixth aspect of the example restricted-access credit device, though the example embodiments are not limited in this respect.

In an eighth aspect of the example restricted-access credit device, the one or more processors are configured to provide indemnity up to a designated amount for at least one of an ambulance trip, an overnight hospital stay, a disease screening, or an emergency room visit based at least in part on payment of a membership fee to use the restricted-access credit device by a user to whom the line of credit is granted. The eighth aspect of the example restricted-access credit device may be implemented in combination with the first, second, third, fourth, fifth, sixth, and/or seventh aspect of the example restricted-access credit device, though the example embodiments are not limited in this respect.

In a ninth aspect of the example restricted-access credit device, the one or more processors are configured to provide indemnity up to a first designated amount for at least one of an ambulance trip, an overnight hospital stay, a disease screening, or an emergency room visit in a first geographic region in which a user to whom the line of credit is granted resides based at least in part on payment of a first membership fee to use the restricted-access credit device by the user and up to a second designated amount for at least one of the ambulance trip, the overnight hospital stay, the disease screening, or the emergency room visit in a second geographic region that is different from the first geographic region based at least in part on payment of a second membership fee that is greater than the first membership fee by the user. The ninth aspect of the example restricted-access credit device may be implemented in combination with the first, second, third, fourth, fifth, sixth, seventh, and/or eighth aspect of the example restricted-access credit device, though the example embodiments are not limited in this respect.

In a tenth aspect of the example restricted-access credit device, the one or more processors are configured to apply at least one of an insurance deductible associated with a health insurance policy of a user to whom the line of credit is granted or a co-payment associated with the health insurance policy against the line of credit. The tenth aspect of the example restricted-access credit device may be implemented in combination with the first, second, third, fourth, fifth, sixth, seventh, eighth, and/or ninth aspect of the example restricted-access credit device, though the example embodiments are not limited in this respect.

In an eleventh aspect of the example restricted-access credit device, the restricted-access credit device is linked to a tax-advantaged health benefit account of a user to whom the line of credit is granted. In accordance with the eleventh aspect, the one or more processors are configured to provide monthly payments from the tax-advantaged health benefit account toward a standing balance resulting from charges applied against the line of credit. The eleventh aspect of the example restricted-access credit device may be implemented in combination with the first, second, third, fourth, fifth, sixth, seventh, eighth, ninth, and/or tenth aspect of the example restricted-access credit device, though the example embodiments are not limited in this respect.

In a twelfth aspect of the example restricted-access credit device, the one or more processors are configured to select one or more healthcare-related items, which are to be recommended to a user to whom the line of credit is granted, from a plurality of healthcare-related items based at least in part on at least one of a purchasing behavior of the user or a location of the user. In accordance with the twelfth aspect, the one or more processors are configured to recommend the one or more healthcare-related items to the user based at least in part on selection of the one or more healthcare-related items. The twelfth aspect of the example restricted-access credit device may be implemented in combination with the first, second, third, fourth, fifth, sixth, seventh, eighth, ninth, tenth, and/or eleventh aspect of the example restricted-access credit device, though the example embodiments are not limited in this respect.

In a thirteenth aspect of the example restricted-access credit device, the one or more processors are configured to select one or more healthcare-related facilities, which are to be recommended to a user to whom the line of credit is granted, from a plurality of healthcare-related facilities based at least in part on at least one of a quality rating of each of the one or more healthcare-related facilities, a cost of one or more services at each of the one or more healthcare-related facilities, or a location of each of the one or more healthcare-related facilities. In accordance with the thirteenth aspect, the one or more processors are configured to recommend the one or more healthcare-related facilities to the user based at least in part on selection of the one or more healthcare-related facilities. The thirteenth aspect of the example restricted-access credit device may be implemented in combination with the first, second, third, fourth, fifth, sixth, seventh, eighth, ninth, tenth, eleventh, and/or twelfth aspect of the example restricted-access credit device, though the example embodiments are not limited in this respect.

In a fourteenth aspect of the example restricted-access credit device, the item includes a medical procedure performed on a user to whom the line of credit is granted. In accordance with the fourteenth aspect, the one or more processors are configured to cause a claim regarding at least one of medical misconduct or medical malpractice to be generated on behalf of the user based at least in part on information regarding damages that the user claims to have occurred as a result of the medical procedure. The fourteenth aspect of the example restricted-access credit device may be implemented in combination with the first, second, third, fourth, fifth, sixth, seventh, eighth, ninth, tenth, eleventh, twelfth, and/or thirteenth aspect of the example restricted-access credit device, though the example embodiments are not limited in this respect.

In an example of the fourteenth aspect, the one or more processors are configured to generate a rating for at least one of a medical facility at which the medical procedure is performed or a doctor who performed the medical procedure based at least in part on the claim.

In a fifteenth aspect of the example restricted-access credit device, the one or more processors are configured to process a death certificate of a user to whom the line of credit is granted. The fifteenth aspect of the example restricted-access credit device may be implemented in combination with the first, second, third, fourth, fifth, sixth, seventh, eighth, ninth, tenth, eleventh, twelfth, thirteenth, and/or fourteenth aspect of the example restricted-access credit device, though the example embodiments are not limited in this respect.

In a sixteenth aspect of the example restricted-access credit device, the one or more processors are further configured to cause an identifier associated with the user to be cross-referenced with a plurality of reference identifiers that are associated with a plurality of respective members of a credit union to determine whether the user is a member of the credit union. The identifier matching at least one of the reference identifiers indicates that the user is a member of the credit union. The identifier not matching at least one of the reference identifiers indicates that the user is not a member of the credit union. In accordance with the fifteenth aspect, the one or more processors are further configured to cause attributes of the user to be compared with criteria for becoming a member of the credit union. In further accordance with the fifteenth aspect, the one or more processors are further configured to selectively solicit the user to join the credit union depending on whether the user is a member of the credit union and further depending on whether the attributes of the user satisfy the criteria. The sixteenth aspect of the example restricted-access credit device may be implemented in combination with the first, second, third, fourth, fifth, sixth, seventh, eighth, ninth, tenth, eleventh, twelfth, thirteenth, fourteenth, and/or fifteenth aspect of the example restricted-access credit device, though the example embodiments are not limited in this respect.

In an example of the sixteenth aspect, the one or more processors are configured to pre-qualify the user to be a member of the credit union based at least in part on the attributes of the user satisfying the criteria. In accordance with this example, the one or more processors are configured to solicit the user to join the credit union based at least in part on the user not being a member of the credit union and further based at least in part on the user being pre-qualified to be a member of the credit union.

In a seventeenth aspect of the example restricted-access credit device, the one or more processors are further configured to perform the following operations for each of a plurality of credit unions: cause an identifier associated with the user to be cross-referenced with a plurality of reference identifiers that are associated with a plurality of respective members of the credit union to determine whether the user is a member of the credit union; and cause attributes of the user to be compared with criteria for becoming a member of the credit union. The identifier matching at least one of the reference identifiers indicates that the user is a member of the credit union. The identifier not matching at least one of the reference identifiers indicates that the user is not a member of the credit union. In accordance with the seventeenth aspect, the one or more processors are further configured to determine a first subset of the plurality of credit unions, wherein the user is not a member of each credit union in the first subset, and wherein the attributes of the user satisfy the criteria for becoming a member of each credit union in the first subset. In further accordance with the seventeenth aspect, the one or more processors are further configured to cause the credit unions in the first subset to be ranked based at least in part on fees that are charged by the credit unions in the first subset. The fees that are charged by a credit union being relatively low corresponds to a relatively high rank of the credit union. The fees that are charged by a credit union being relatively high corresponds to a relatively low rank of the credit union. In further accordance with the seventeenth aspect, the one or more processors are further configured to cause a second subset of the plurality of credit unions to be selected from the first subset based at least in part on each credit union in the second subset having a rank that is greater than or equal to a threshold rank. In further accordance with the seventeenth aspect, the one or more processors are further configured to cause a notice to be presented via a user interface that is included in the point-of-sale terminal. The notice identifies each credit union in the second subset and informs the user that the user is eligible for membership in the respective credit union. The seventeenth aspect of the example restricted-access credit device may be implemented in combination with the first, second, third, fourth, fifth, sixth, seventh, eighth, ninth, tenth, eleventh, twelfth, thirteenth, fourteenth, fifteenth, and/or sixteenth aspect of the example restricted-access credit device, though the example embodiments are not limited in this respect.

In an example method of restricting access to a line of credit associated with a restricted-access credit device, communication is performed with a point-of-sale terminal by providing an account identifier that identifies the line of credit associated with the restricted-access credit device to the point-of-sale terminal and further by receiving a purchase request that requests execution of a purchase transaction for an item from the point-of-sale terminal, the item including at least one of one or more goods or one or more services. A merchant category code of a merchant that is associated with the point-of-sale terminal is identified by analyzing the purchase request. The merchant category code of the merchant is caused to be cross-referenced with a plurality of healthcare-related merchant category codes. Execution of the purchase transaction is selectively triggered depending on whether the merchant category code of the merchant corresponds to at least one of the healthcare-related merchant category codes. The selectively triggering execution of the purchase transaction comprises triggering execution of the purchase transaction based at least in part on the merchant category code of the merchant corresponding to at least one of the healthcare-related merchant category codes, or not triggering execution of the purchase transaction based at least in part on the merchant category code of the merchant not corresponding to at least one of the healthcare-related merchant category codes.

In a first aspect of the example method, communicating with the point-of-sale terminal comprises communicating with the point-of-sale terminal further by receiving a stock keeping unit (SKU) associated with the item from the point-of-sale terminal. The SKU is configured to distinguish an item type of the item from other item types of other items. In accordance with the first aspect, selectively triggering execution of the purchase transaction comprises selectively triggering execution of the purchase transaction further depending on whether the SKU associated with the item corresponds to at least one of a plurality of pre-approved stock keeping units.

In a second aspect of the example method, communicating with the point-of-sale terminal comprises communicating with the point-of-sale terminal further by receiving a stock keeping unit (SKU) associated with the item from the point-of-sale terminal. The SKU is configured to distinguish an item type of the item from other item types of other items. In accordance with the second aspect, selectively triggering execution of the purchase transaction comprises selectively triggering execution of the purchase transaction further depending on whether the SKU is associated with a healthcare-related item. The second aspect of the example method may be implemented in combination with the first aspect of the example method, though the example embodiments are not limited in this respect.

In a first example of the second aspect, the example method further comprises storing information regarding an incentive offered by an employer of a user to whom the line of credit is granted, the incentive offering a credit based at least in part on a purchase of the item from the merchant that is associated with the point-of-sale terminal rather than from another merchant that offers the item for sale. In accordance with the first example of the second aspect, the example method further comprises applying the credit to the line of credit based at least in part on the user purchasing the item from the merchant rather than from another merchant that offers the item for sale.

In a second example of the second aspect, the example method further comprises storing information regarding an incentive offered by an employer of a user to whom the line of credit is granted, the incentive offering a credit based at least in part on a purchase of the item from the merchant that is associated with the point-of-sale terminal rather than from another merchant that offers the item for sale. In accordance with the second example of the second aspect, the example method further comprises applying the credit toward an insurance deductible associated with a health insurance policy of the user based at least in part on the user purchasing the item from the merchant rather than from another merchant that offers the item for sale.

In a third aspect of the example method, the restricted-access credit device is linked to a tax-advantaged health benefit account of a user to whom the line of credit is granted. The tax-advantaged health benefit account has funds available for purchases. In accordance with the third aspect, selectively triggering execution of the purchase transaction comprises triggering execution of the purchase transaction by causing the funds of the tax-advantaged health benefit account to be applied toward a cost of the item. The third aspect of the example method may be implemented in combination with the first and/or second aspect of the example method, though the example embodiments are not limited in this respect.

In a fourth aspect of the example method, the example method further comprises downloading a transaction history, which identifies items purchased with the restricted-access credit device, from a memory that is remote from the restricted-access credit device to the memory that is included in the restricted-access credit device. The fourth aspect of the example method may be implemented in combination with the first, second, and/or third aspect of the example method, though the example embodiments are not limited in this respect.

In an example of the fourth aspect, the example method further comprises preparing an annual tax statement of a user to whom the line of credit is granted based at least in part on the transaction history.

In a fifth aspect of the example method, the example method further comprises storing a policy that identifies the plurality of healthcare-related merchant category codes in a memory of the restricted-access credit device. In accordance with the fifth aspect, selectively triggering execution of the purchase transaction comprises enforcing the policy by selectively triggering execution of the purchase transaction depending on whether the merchant category code of the merchant corresponds to at least one of the healthcare-related merchant category codes. The fifth aspect of the example method may be implemented in combination with the first, second, third, fourth, and/or fifth aspect of the example method, though the example embodiments are not limited in this respect.

In a sixth aspect of the example method, the example method further comprises assigning a plurality of relative priorities to a plurality of respective types of healthcare-related items. In accordance with the sixth aspect, the example method further comprises receiving a request for additional credit to be added to the line of credit associated with the restricted-access credit device to cover cost of a medical expense that exceeds an amount of credit available through the line of credit. In further accordance with the sixth aspect, the example method further comprises determining whether the additional credit is to be added to the line of credit by performing operations comprising: causing information regarding a user to whom the line of credit is granted to be analyzed to determine a creditworthiness rating of the user; and analyzing information regarding an item with which the medical expense is associated to determine the type of the item with which the medical expense is associated. In further accordance with the sixth aspect, the example method further comprises adding the additional credit to the line of credit to cover the cost of the medical expense based at least in part on the creditworthiness rating of the user being greater than or equal to a creditworthiness threshold and further based at least in part on the relative priority of the type of the item with which the medical expense is associated being greater than or equal to a priority threshold. The sixth aspect of the example method may be implemented in combination with the first, second, third, fourth, fifth, and/or sixth aspect of the example method, though the example embodiments are not limited in this respect.

In a seventh aspect of the example method, the example method further comprises providing indemnity up to a designated amount for at least one of an ambulance trip, an overnight hospital stay, a disease screening, or an emergency room visit based at least in part on payment of a membership fee to use the restricted-access credit device by a user to whom the line of credit is granted. The seventh aspect of the example method may be implemented in combination with the first, second, third, fourth, fifth, and/or sixth aspect of the example method, though the example embodiments are not limited in this respect.

In an eighth aspect of the example method, the example method further comprises providing indemnity up to a first designated amount for at least one of an ambulance trip, an overnight hospital stay, a disease screening, or an emergency room visit in a first geographic region in which a user to whom the line of credit is granted resides based at least in part on payment of a first membership fee to use the restricted-access credit device by the user. In accordance with the eighth aspect, the example method further comprises providing indemnity up to a second designated amount for at least one of the ambulance trip, the overnight hospital stay, the disease screening, or the emergency room visit in a second geographic region that is different from the first geographic region based at least in part on payment of a second membership fee that is greater than the first membership fee by the user. The eighth aspect of the example method may be implemented in combination with the first, second, third, fourth, fifth, sixth, and/or seventh aspect of the example method, though the example embodiments are not limited in this respect.

In a ninth aspect of the example method, the example method further comprises applying at least one of an insurance deductible associated with a health insurance policy of a user to whom the line of credit is granted or a co-payment associated with the health insurance policy against the line of credit. The ninth aspect of the example method may be implemented in combination with the first, second, third, fourth, fifth, sixth, seventh, and/or eighth aspect of the example method, though the example embodiments are not limited in this respect.

In a tenth aspect of the example method, the restricted-access credit device is linked to a tax-advantaged health benefit account of a user to whom the line of credit is granted. In accordance with the tenth aspect, the example method comprises providing monthly payments from the tax-advantaged health benefit account toward a standing balance resulting from charges applied against the line of credit. The tenth aspect of the example method may be implemented in combination with the first, second, third, fourth, fifth, sixth, seventh, eighth, and/or ninth aspect of the example method, though the example embodiments are not limited in this respect.

In an eleventh aspect of the example method, the example method further comprises selecting one or more healthcare-related items, which are to be recommended to a user to whom the line of credit is granted, from a plurality of healthcare-related items based at least in part on at least one of a purchasing behavior of the user or a location of the user. In accordance with the eleventh aspect, the example method further comprises recommending the one or more healthcare-related items to the user based at least in part on selection of the one or more healthcare-related items. The eleventh aspect of the example method may be implemented in combination with the first, second, third, fourth, fifth, sixth, seventh, eighth, ninth, and/or tenth aspect of the example method, though the example embodiments are not limited in this respect.

In a twelfth aspect of the example method, the example method further comprises selecting one or more healthcare-related facilities, which are to be recommended to a user to whom the line of credit is granted, from a plurality of healthcare-related facilities based at least in part on at least one of a quality rating of each of the one or more healthcare-related facilities, a cost of one or more services at each of the one or more healthcare-related facilities, or a location of each of the one or more healthcare-related facilities. In accordance with the twelfth aspect, the example method further comprises recommending the one or more healthcare-related facilities to the user based at least in part on selection of the one or more healthcare-related facilities. The twelfth aspect of the example method may be implemented in combination with the first, second, third, fourth, fifth, sixth, seventh, eighth, ninth, tenth, and/or eleventh aspect of the example method, though the example embodiments are not limited in this respect.

In an example of the twelfth aspect, the example method further comprises generating a rating for at least one of a medical facility at which the medical procedure is performed or a doctor who performed the medical procedure based at least in part on the claim.

In a thirteenth aspect of the example method, the example method further comprises processing a death certificate of a user to whom the line of credit is granted. The thirteenth aspect of the example method may be implemented in combination with the first, second, third, fourth, fifth, sixth, seventh, eighth, ninth, tenth, eleventh, and/or twelfth aspect of the example method, though the example embodiments are not limited in this respect.

In a fourteenth aspect of the example method, the example method further comprises causing an identifier associated with the user to be cross-referenced with a plurality of reference identifiers that are associated with a plurality of respective members of a credit union to determine whether the user is a member of the credit union. The identifier matching at least one of the reference identifiers indicates that the user is a member of the credit union. The identifier not matching at least one of the reference identifiers indicates that the user is not a member of the credit union. In accordance with the fourteenth aspect, the example method further comprises causing attributes of the user to be compared with criteria for becoming a member of the credit union. In further accordance with the fourteenth aspect, the example method further comprises selectively soliciting the user to join the credit union depending on whether the user is a member of the credit union and further depending on whether the attributes of the user satisfy the criteria. The fourteenth aspect of the example method may be implemented in combination with the first, second, third, fourth, fifth, sixth, seventh, eighth, ninth, tenth, eleventh, twelfth, and/or thirteenth aspect of the example method, though the example embodiments are not limited in this respect.

In an example of the fourteenth aspect, the example method further comprises pre-qualifying the user to be a member of the credit union based at least in part on the attributes of the user satisfying the criteria. In accordance with this example, selectively soliciting the user to join the credit union comprises soliciting the user to join the credit union based at least in part on the user not being a member of the credit union and further based at least in part on the user being pre-qualitied to be a member of the credit union.

In a fifteenth aspect of the example method, the example method further comprises performing the following operations for each of a plurality of credit unions: causing an identifier associated with the user to be cross-referenced with a plurality of reference identifiers that are associated with a plurality of respective members of the credit union to determine whether the user is a member of the credit union; and causing attributes of the user to be compared with criteria for becoming a member of the credit union. The identifier matching at least one of the reference identifiers indicates that the user is a member of the credit union. The identifier not matching at least one of the reference identifiers indicates that the user is not a member of the credit union. In accordance with the fifteenth aspect, the example method further comprises determining a first subset of the plurality of credit unions, wherein the user is not a member of each credit union in the first subset, and wherein the attributes of the user satisfy the criteria for becoming a member of each credit union in the first subset. In further accordance with the fifteenth aspect, the example method further comprises causing the credit unions in the first subset to be ranked based at least in part on fees that are charged by the credit unions in the first subset. The fees that are charged by a credit union being relatively low corresponds to a relatively high rank of the credit union. The fees that are charged by a credit union being relatively high corresponds to a relatively low rank of the credit union. In further accordance with the fifteenth aspect, the example method further comprises causing a second subset of the plurality of credit unions to be selected from the first subset based at least in part on each credit union in the second subset having a rank that is greater than or equal to a threshold rank. In further accordance with the fifteenth aspect, the example method further comprises causing a notice to be presented via a user interface that is included in the point-of-sale terminal, the notice identifying each credit union in the second subset and informing the user that the user is eligible for membership in the respective credit union. The fifteenth aspect of the example method may be implemented in combination with the first, second, third, fourth, fifth, sixth, seventh, eighth, ninth, tenth, eleventh, twelfth, thirteenth, and/or fourteenth aspect of the example method, though the example embodiments are not limited in this respect.

Another example device comprises memory and one or more processors coupled to the memory. The one or more processors are configured to establish communication with a point-of-sale reader on behalf of a user. The communication initiates a purchase of at least one of one or more goods or one or more services. The one or more processors are further configured to authenticate the user to a store that stores aggregated healthcare data associated with the user by providing credentials of the user to the store. The one or more processors are further configured to obtain access to the aggregated healthcare data, which includes portions of the aggregated healthcare data that are aggregated from respective healthcare data storage systems that do not share the respective portions of the aggregated healthcare data with others of the healthcare data storage systems, via the point-of-sale reader, based at least in part on the credentials of the user and reference credentials being same. The one or more processors are further configured to cause at least some of the aggregated healthcare data to be presented via a user interface that is included in the point-of-sale reader based at least in part on access to the aggregated healthcare data being obtained.

In a first aspect of the example device, the one or more processors include a first processor and a second processor. In accordance with the first aspect, the device comprises a first chip that includes the first processor, which is configured to cause the purchase of the at least one of the one or more goods or the one or more services to be initiated by establishing the communication with the point-of-sale reader; and a second chip that includes the second processor, which is configured to obtain the access to the aggregated healthcare data associated with the user. The second chip is different from the first chip.

In a second aspect of the example device, at least one processor of the one or more processors is incorporated into a single chip. In accordance with the second aspect, the at least one processor is configured to establish the communication with the point-of-sale reader and to obtain the access to the aggregated healthcare data associated with the user. The second aspect of the example device may be implemented in combination with the first aspect of the example device, though the example embodiments are not limited in this respect.

In a third aspect of the example device, the one or more processors are configured to obtain the access to the aggregated healthcare data associated with the user by downloading the aggregated healthcare data through the point-of-sale reader. The third aspect of the example device may be implemented in combination with the first and/or second aspect of the example device, though the example embodiments are not limited in this respect.

In a fourth aspect of the example device, the one or more processors are further configured to upload information regarding the at least one of the one or more goods or the one or more services to each of the healthcare data storage systems through the point-of-sale reader. The fourth aspect of the example device may be implemented in combination with the first, second, and/or third aspect of the example device, though the example embodiments are not limited in this respect.

In a fifth aspect of the example device, the one or more processors are further configured to store a first portion of the aggregated healthcare data, which is downloaded from a first healthcare data storage system, in the memory based at least in part on the access to the aggregated healthcare data being obtained. In accordance with the fifth aspect, the one or more processors are further configured to upload the first portion of the aggregated healthcare data to a second healthcare data storage system that is different from the first healthcare data storage system. In further accordance with the fifth aspect, the one or more processors are further configured to delete the first portion of the aggregated healthcare data from the memory based at least in part on the first portion of the aggregated healthcare data being uploaded to the second healthcare data storage system. The fifth aspect of the example device may be implemented in combination with the first, second, third, and/or fourth aspect of the example device, though the example embodiments are not limited in this respect.

In a sixth aspect of the example device, the at least one of the one or more goods or the one or more services includes at least one of one or more healthcare-related goods or one or more healthcare-related services. In accordance with the sixth aspect, the one or more processors are configured to aggregate the portions of the aggregated healthcare data from the respective healthcare data storage systems by uploading the portions to the store, including upload information relating to the purchase of the at least one of the one or more healthcare-related goods or the one or more healthcare-related services via the point-of-sale reader to the store. In further accordance with the sixth aspect, the one or more processors are configured to cause the at least some of the aggregated healthcare data that is different from the information relating to the purchase of the at least one of the one or more healthcare-related goods or the one or more healthcare-related services to be presented via the user interface that is included in the point-of-sale reader based at least in part on the access to the aggregated healthcare data being obtained. The sixth aspect of the example device may be implemented in combination with the first, second, third, fourth, and/or fifth aspect of the example device, though the example embodiments are not limited in this respect.

In a seventh aspect of the example device, the at least one of the one or more goods or the one or more services includes a medication. In accordance with the seventh aspect, the at least some of the aggregated healthcare data includes information specifying a potential consequence of mixing the medication with one or more other substances. The seventh aspect of the example device may be implemented in combination with the first, second, third, fourth, fifth, and/or sixth aspect of the example device, though the example embodiments are not limited in this respect.

In an eighth aspect of the example device, the at least one of the one or more goods or the one or more services includes a first medication and a second medication. In accordance with the eighth aspect, the at least some of the aggregated healthcare data includes information specifying a potential consequence of mixing the first medication and the second medication based at least in part on the first medication and the second medication being purchased in a common transaction. The eighth aspect of the example device may be implemented in combination with the first, second, third, fourth, fifth, sixth, and/or seventh aspect of the example device, though the example embodiments are not limited in this respect.

In a ninth aspect of the example device, the at least one of the one or more goods or the one or more services includes at least one of one or more healthcare-related goods or one or more healthcare-related services. In accordance with the ninth aspect, the at least some of the aggregated healthcare data includes information specifying an authorized amount that a provider is authorized to charge for the at least one of the one or more healthcare-related goods or the one or more healthcare-related services based at least in part on the purchase of the at least one of the one or more healthcare-related goods or the one or more healthcare-related services being initiated. The ninth aspect of the example device may be implemented in combination with the first, second, third, fourth, fifth, sixth, seventh, and/or eighth aspect of the example device, though the example embodiments are not limited in this respect.

In a tenth aspect of the example device, the one or more processors are configured to generate a cryptographic record of a plurality of purchases, which are initiated via a plurality of respective communications that are established with one or more point-of-sale readers and which include the purchase that is initiated by the communication with the point-of-sale reader, using blockchain technology. In accordance with the tenth aspect, the cryptographic record of the plurality of purchases is based on an order of the plurality of purchases. The tenth aspect of the example device may be implemented in combination with the first, second, third, fourth, fifth, sixth, seventh, eighth, and/or ninth aspect of the example device, though the example embodiments are not limited in this respect.

In an eleventh aspect of the example device, the one or more processors are configured to communicate with the point-of-sale reader by providing an account identifier that identifies a line of credit associated with the device to the point-of-sale reader and further by receiving a purchase request that requests execution of a purchase transaction corresponding to the purchase from the point-of-sale reader, the purchase request including a merchant category code of a merchant that is associated with the point-of-sale reader. In accordance with the eleventh aspect, the one or more processors are configured to cause the merchant category code of the merchant to be cross-referenced with a plurality of healthcare-related merchant category codes. In accordance with the eleventh aspect, the one or more processors are configured to selectively trigger execution of the purchase transaction depending on whether the merchant category code of the merchant corresponds to at least one of the healthcare-related merchant category codes. The eleventh aspect of the example device may be implemented in combination with the first, second, third, fourth, fifth, sixth, seventh, eighth, ninth, and/or tenth aspect of the example device, though the example embodiments are not limited in this respect.

In a twelfth aspect of the example device, the device is associated with a restricted-access credit device, which is external to the device. In accordance with the twelfth aspect, the one or more processors are further configured to cause a portion of a cost of the at least one of the one or more goods or the one or more services that is not covered by a healthcare plan to be applied against a line of credit associated with the restricted-access credit device. The twelfth aspect of the example device may be implemented in combination with the first, second, third, fourth, fifth, sixth, seventh, eighth, ninth, tenth, and/or eleventh aspect of the example device, though the example embodiments are not limited in this respect.

In a thirteenth aspect of the example device, the user is non-human animal. In accordance with the thirteenth aspect, the communication initiates the purchase of the at least one of the one or more goods or the one or more services for the non-human animal. The thirteenth aspect of the example device may be implemented in combination with the first, second, third, fourth, fifth, sixth, seventh, eighth, ninth, tenth, eleventh, and/or twelfth aspect of the example device, though the example embodiments are not limited in this respect.

In an example of the thirteenth aspect, the device is implanted under skin of the non-human animal.

In a fourteenth aspect of the example device, the user is non-human animal. In accordance with the fourteenth aspect, the one or more processors are configured to cause a bloodline of the non-human animal to be compared to a plurality of bloodlines that are identified in a bloodline database that is included in the store to identify the bloodline of the non-human animal in the bloodline database. In accordance with the fourteenth aspect, the one or more processors are configured to cause at least one of one or more illnesses or one or more infectious diseases that occur with regard to the bloodline of the non-human animal to be identified based at least in part on the at least one of the one or more illnesses or the one or more infectious diseases being cross-referenced with the bloodline of the non-human animal in the bloodline database. In further accordance with the fourteenth aspect, the one or more processors are configured to cause an identity of the at least one of the one or more illnesses or the one or more infectious diseases to be presented via the user interface that is included in the point-of-sale reader. The fourteenth aspect of the example device may be implemented in combination with the first, second, third, fourth, fifth, sixth, seventh, eighth, ninth, tenth, eleventh, twelfth, and/or thirteenth aspect of the example device, though the example embodiments are not limited in this respect.

In an example method of accessing healthcare data using a healthcare data chip device, communication is established with a point-of-sale reader on behalf of a user. The communication initiates a purchase of at least one of one or more goods or one or more services. The user is authenticated to a store that stores aggregated healthcare data associated with the user by providing credentials of the user to the store. Access to the aggregated healthcare data, which includes portions of the aggregated healthcare data that are aggregated from respective healthcare data storage systems that do not share the respective portions of the aggregated healthcare data with others of the healthcare data storage systems, is obtained via the point-of-sale reader, based at least in part on the credentials of the user and reference credentials being same. At least some of the aggregated healthcare data is caused to be presented via a user interface that is included in the point-of-sale reader based at least in part on access to the aggregated healthcare data being obtained.

In a first aspect of the example method, establishing the communication comprises causing the purchase of the at least one of the one or more goods or the one or more services to be initiated using a first processor that is included in a first chip by establishing the communication with the point-of-sale reader. In accordance with the first aspect, obtaining the access to the aggregated healthcare data comprises obtaining the access to the aggregated healthcare data associated with the user using a second processor that is included in a second chip that is different from the first chip.

In a second aspect of the example method, establishing the communication with the point-of-sale reader and obtaining the access to the aggregated healthcare data associated with the user are performed using a common computer chip. The second aspect of the example method may be implemented in combination with the first aspect of the example method, though the example embodiments are not limited in this respect.

In a third aspect of the example method, obtaining the access to the aggregated healthcare data comprises downloading the aggregated healthcare data associated with the user through the point-of-sale reader. The third aspect of the example method may be implemented in combination with the first and/or second aspect of the example method, though the example embodiments are not limited in this respect.

In a fourth aspect of the example method, the example method further comprises uploading information regarding the at least one of the one or more goods or the one or more services to each of the healthcare data storage systems through the point-of-sale reader. The fourth aspect of the example method may be implemented in combination with the first, second, and/or third aspect of the example method, though the example embodiments are not limited in this respect.

In a fifth aspect of the example method, obtaining the access to the aggregated healthcare data comprises downloading a first portion of the aggregated healthcare data from a first healthcare data storage system to the healthcare data chip device. In accordance with the fifth aspect, the example method further comprises storing the first portion of the aggregated healthcare data in a memory of the healthcare data chip device based at least in part on the access to the aggregated healthcare data being obtained. In further accordance with the fifth aspect, the example method further comprises uploading the first portion of the aggregated healthcare data from the memory to a second healthcare data storage system that is different from the first healthcare data storage system. In further accordance with the fifth aspect, the example method further comprises deleting the first portion of the aggregated healthcare data from the memory based at least in part on the first portion of the aggregated healthcare data being uploaded to the second healthcare data storage system. The fifth aspect of the example method may be implemented in combination with the first, second, third, and/or fourth aspect of the example method, though the example embodiments are not limited in this respect.

In a sixth aspect of the example method, the at least one of the one or more goods or the one or more services includes at least one of one or more healthcare-related goods or one or more healthcare-related services. In accordance with the sixth aspect, the example method further comprises aggregating the portions of the aggregated healthcare data from the respective healthcare data storage systems by uploading the portions to the store, including uploading information relating to the purchase of the at least one of the one or more healthcare-related goods or the one or more healthcare-related services via the point-of-sale reader to the store. In further accordance with the sixth aspect, causing the at least some of the aggregated healthcare data to be presented via the user interface comprises causing the at least some of the aggregated healthcare data that is different from the information relating to the purchase of the at least one of the one or more healthcare-related goods or the one or more healthcare-related services to be presented via the user interface that is included in the point-of-sale reader based at least in part on the access to the aggregated healthcare data being obtained. The sixth aspect of the example method may be implemented in combination with the first, second, third, fourth, and/or fifth aspect of the example method, though the example embodiments are not limited in this respect.

In a seventh aspect of the example method, the at least one of the one or more goods or the one or more services includes a medication. In accordance with the seventh aspect, the at least some of the aggregated healthcare data includes information specifying a potential consequence of mixing the medication with one or more other substances. The seventh aspect of the example method may be implemented in combination with the first, second, third, fourth, fifth, and/or sixth aspect of the example method, though the example embodiments are not limited in this respect.

In an eighth aspect of the example method, the at least one of the one or more goods or the one or more services includes a first medication and a second medication. In accordance with the eighth aspect, the at least some of the aggregated healthcare data includes information specifying a potential consequence of mixing the first medication and the second medication based at least in part on the first medication and the second medication being purchased in a common transaction. The eighth aspect of the example method may be implemented in combination with the first, second, third, fourth, fifth, sixth, and/or seventh aspect of the example method, though the example embodiments are not limited in this respect.

In a ninth aspect of the example method, the at least one of the one or more goods or the one or more services includes at least one of one or more healthcare-related goods or one or more healthcare-related services. In accordance with the ninth aspect, the at least some of the aggregated healthcare data includes information specifying an authorized amount that a provider is authorized to charge for the at least one of the one or more healthcare-related goods or the one or more healthcare-related services based at least in part on the purchase of the at least one of the one or more healthcare-related goods or the one or more healthcare-related services being initiated. The ninth aspect of the example method may be implemented in combination with the first, second, third, fourth, fifth, sixth, seventh, and/or eighth aspect of the example method, though the example embodiments are not limited in this respect.

In a tenth aspect of the example method, the example method further comprises generating a cryptographic record of a plurality of purchases, which are initiated via a plurality of respective communications that are established with one or more point-of-sale readers and which include the purchase that is initiated by the communication with the point-of-sale reader, using blockchain technology. In accordance with the tenth aspect, the cryptographic record of the plurality of purchases is based on an order of the plurality of purchases. The tenth aspect of the example method may be implemented in combination with the first, second, third, fourth, fifth, sixth, seventh, eighth, and/or ninth aspect of the example method, though the example embodiments are not limited in this respect.

In an eleventh aspect of the example method, the example method comprises communicating with the point-of-sale reader by providing an account identifier that identifies a line of credit associated with the healthcare data chip device to the point-of-sale reader and further by receiving a purchase request that requests execution of a purchase transaction corresponding to the purchase from the point-of-sale reader, the purchase request including a merchant category code of a merchant that is associated with the point-of-sale reader. In accordance with the eleventh aspect, the example method comprises causing the merchant category code of the merchant to be cross-referenced with a plurality of healthcare-related merchant category codes. In further accordance with the eleventh aspect, the example method comprises selectively triggering execution of the purchase transaction depending on whether the merchant category code of the merchant corresponds to at least one of the healthcare-related merchant category codes. The eleventh aspect of the example method may be implemented in combination with the first, second, third, fourth, fifth, sixth, seventh, eighth, ninth, and/or tenth aspect of the example method, though the example embodiments are not limited in this respect.

In a twelfth aspect of the example method, the healthcare data chip device is associated with a restricted-access credit device that is external to the healthcare data chip device. In accordance with the twelfth aspect, the example method further comprises causing, by the healthcare data chip device, a portion of a cost of the at least one of the one or more goods or the one or more services that is not covered by a healthcare plan to be applied against a line of credit associated with the restricted-access credit device. The twelfth aspect of the example method may be implemented in combination with the first, second, third, fourth, fifth, sixth, seventh, eighth, ninth, tenth, and/or eleventh aspect of the example method, though the example embodiments are not limited in this respect.

In a thirteenth aspect of the example method, the user is non-human animal. In accordance with the thirteenth aspect, the communication initiates the purchase of the at least one of the one or more goods or the one or more services for the non-human animal. The thirteenth aspect of the example method may be implemented in combination with the first, second, third, fourth, fifth, sixth, seventh, eighth, ninth, tenth, eleventh, and/or twelfth aspect of the example method, though the example embodiments are not limited in this respect.

In an example of the thirteenth aspect, the healthcare data chip device that performs the method is implanted under skin of the non-human animal.

In a fourteenth aspect of the example method, the user is non-human animal. In accordance with the fourteenth aspect, the example method further comprises causing a bloodline of the non-human animal to be compared to a plurality of bloodlines that are identified in a bloodline database that is included in the store to identify the bloodline of the non-human animal in the bloodline database. In further accordance with the fourteenth aspect, the example method further comprises causing at least one of one or more illnesses or one or more infectious diseases that occur with regard to the bloodline of the non-human animal to be identified based at least in part on the at least one of the one or more illnesses or the one or more infectious diseases being cross-referenced with the bloodline of the non-human animal in the bloodline database. In further accordance with the fourteenth aspect, causing the at least some of the aggregated healthcare data to be presented via the user interface comprises causing an identity of the at least one of the one or more illnesses or the one or more infectious diseases to be presented via the user interface that is included in the point-of-sale reader. The fourteenth aspect of the example method may be implemented in combination with the first, second, third, fourth, fifth, sixth, seventh, eighth, ninth, tenth, eleventh, twelfth, and/or thirteenth aspect of the example method, though the example embodiments are not limited in this respect.

A first example computer program product comprises a non-transitory computer-readable medium having computer program logic recorded thereon for enabling a processor-based system to perform operations to restrict access to a line of credit associated with a restricted-access credit device. The operations comprise communicate with a point-of-sale terminal by providing an account identifier that identifies the line of credit associated with the restricted-access credit device to the point-of-sale terminal and further by receiving a purchase request that requests execution of a purchase transaction for an item from the point-of-sale terminal. The purchase request includes a merchant category code of a merchant that is associated with the point-of-sale terminal. The item includes at least one of one or more goods or one or more services. The operations further comprise cause the merchant category code of the merchant to be cross-referenced with a plurality of healthcare-related merchant category codes. The operations further comprise selectively trigger execution of the purchase transaction depending on whether the merchant category code of the merchant corresponds to at least one of the healthcare-related merchant category codes, including: trigger execution of the purchase transaction based at least in part on the merchant category code of the merchant corresponding to at least one of the healthcare-related merchant category codes, or not trigger execution of the purchase transaction based at least in part on the merchant category code of the merchant not corresponding to at least one of the healthcare-related merchant category codes.

A second example computer program product comprises a computer-readable storage medium having instructions recorded thereon for enabling a processor-based system to perform operations. The operations comprise establishing communication with a point-of-sale reader on behalf of a user. The communication initiates a purchase of at least one of one or more goods or one or more services. The operations further comprise authenticating the user to a store that stores aggregated healthcare data associated with the user by providing credentials of the user to the store. The operations further comprise obtaining access to the aggregated healthcare data, which includes portions of the aggregated healthcare data that are aggregated from respective healthcare data storage systems that do not share the respective portions of the aggregated healthcare data with others of the healthcare data storage systems, via the point-of-sale reader, based at least in part on the credentials of the user and reference credentials being same. The operations further comprise causing at least some of the aggregated healthcare data to be presented via a user interface that is included in the point-of-sale reader based at least in part on access to the aggregated healthcare data being obtained.

### IV. Example Computer System

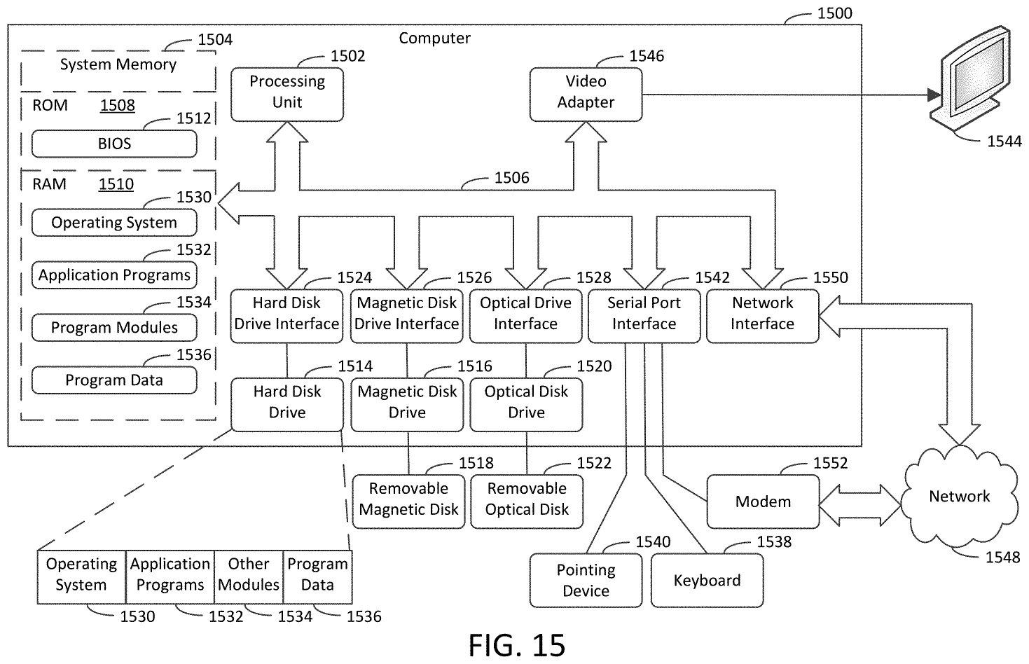

FIG. 15 depicts an example computer 1500 in which embodiments may be implemented. The restricted-access credit device 102, the POS terminal 104, the cash register 106, the item scanner 108, the server 110, the restricted-access credit device 400, the restricted-access credit device 700, the healthcare data chip device 802, the POS terminal 804, the cash register 806, the item scanner 808, the server 810, and/or the healthcare data chip device 1200 may be implemented using computer 1500, including one or more features of computer 1500 and/or alternative features. Computer 1500 may be a general-purpose computing device in the form of a conventional personal computer, a mobile computer, or a workstation, for example, or computer 1500 may be a special purpose computing device. For instance, computer 1500 may be a desktop computer, a laptop computer, a tablet computer, a wearable computer such as a smart watch or a head-mounted computer, a personal digital assistant, a cellular telephone, an Internet of things (IoT) device, or the like. The description of computer 1500 provided herein is provided for purposes of illustration, and is not intended to be limiting. Embodiments may be implemented in further types of computer systems, as would be known to persons skilled in the relevant art(s).

As shown in FIG. 15, computer 1500 includes a processing unit 1502, a system memory 1504, and a bus 1506 that couples various system components including system memory 1504 to processing unit 1502. Bus 1506 represents one or more of any of several types of bus structures, including a memory bus or memory controller, a peripheral bus, an accelerated graphics port, and a processor or local bus using any of a variety of bus architectures. System memory 1504 includes read only memory (ROM) 1508 and random access memory (RAM) 1510. A basic input/output system 1512 (BIOS) is stored in ROM 1508.

Computer 1500 also has one or more of the following drives: a hard disk drive 1514 for reading from and writing to a hard disk, a magnetic disk drive 1516 for reading from or writing to a removable magnetic disk 1518, and an optical disk drive 1520 for reading from or writing to a removable optical disk 1522 such as a CD ROM, DVD ROM, or other optical media. Hard disk drive 1514, magnetic disk drive 1516, and optical disk drive 1520 are connected to bus 1506 by a hard disk drive interface 1524, a magnetic disk drive interface 1526, and an optical drive interface 1528, respectively. The drives and their associated computer-readable storage media provide nonvolatile storage of computer-readable instructions, data structures, program modules and other data for the computer. Although a hard disk, a removable magnetic disk and a removable optical disk are described, other types of computer-readable storage media can be used to store data, such as flash memory cards, digital video disks, random access memories (RAMs), read only memories (ROM), and the like.

A number of program modules may be stored on the hard disk, magnetic disk, optical disk, ROM, or RAM. These programs include an operating system 1530, one or more application programs 1532, other program modules 1534, and program data 1536. Application programs 1532 or program modules 1534 may include, for example, computer program logic for implementing any one or more of the healthcare-related restriction logic 112, the identification logic 404, the cross-reference logic 406, the credit logic 408, the execution logic 410, the healthcare-related restriction logic 412, the rating logic 414, the information logic 416, the identification logic 704, the healthcare-related restriction logic 712, the comparison logic 762, the solicitation logic 764, the ranking logic 766, the communication logic 1266, the blockchain logic 1268, the authentication logic 1270, the presentation logic 1272, the upload logic 1274, the identification logic 1304, the cross-reference logic 1306, the credit logic 1308, the execution logic 1310, flowchart 200 (including any step of flowchart 200), flowchart 300 (including any step of flowchart 300), flowchart 500 (including any step of flowchart 500), flowchart 600 (including any step of flowchart 600), flowchart 900 (including any step of flowchart 900), flowchart 1000 (including any step of flowchart 1000), and/or flowchart 1100 (including any step of flowchart 1100), as described herein.

A user may enter commands and information into the computer 1500 through input devices such as keyboard 1538 and pointing device 1540. Other input devices (not shown) may include a microphone, joystick, game pad, satellite dish, scanner, touch screen, camera, accelerometer, gyroscope, or the like. These and other input devices are often connected to the processing unit 1502 through a serial port interface 1542 that is coupled to bus 1506, but may be connected by other interfaces, such as a parallel port, game port, or a universal serial bus (USB).

A display device 1544 (e.g., a monitor) is also connected to bus 1506 via an interface, such as a video adapter 1546. In addition to display device 1544, computer 1500 may include other peripheral output devices (not shown) such as speakers and printers.

Computer 1500 is connected to a network 1548 (e.g., the Internet) through a network interface or adapter 1550, a modem 1552, or other means for establishing communications over the network. Modem 1552, which may be internal or external, is connected to bus 1506 via serial port interface 1542.

As used herein, the terms “computer program medium” and “computer-readable storage medium” are used to generally refer to media (e.g., non-transitory media) such as the hard disk associated with hard disk drive 1514, removable magnetic disk 1518, removable optical disk 1522, as well as other media such as flash memory cards, digital video disks, random access memories (RAMs), read only memories (ROM), and the like. A computer-readable storage medium is not a signal, such as a carrier signal or a propagating signal. For instance, a computer-readable storage medium may not include a signal. Accordingly, a computer-readable storage medium does not constitute a signal per se. Computer-readable storage media are distinguished from and non-overlapping with communication media (do not include communication media). Communication media embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media. Example embodiments are also directed to such communication media.

As noted above, computer programs and modules (including application programs 1532 and other program modules 1534) may be stored on the hard disk, magnetic disk, optical disk, ROM, or RAM. Such computer programs may also be received via network interface 1550 or serial port interface 1542. Such computer programs, when executed or loaded by an application, enable computer 1500 to implement features of embodiments discussed herein. Accordingly, such computer programs represent controllers of the computer 1500.

Example embodiments are also directed to computer program products comprising software (e.g., computer-readable instructions) stored on any computer-useable medium. Such software, when executed in one or more data processing devices, causes data processing device(s) to operate as described herein. Embodiments may employ any computer-useable or computer-readable medium, known now or in the future. Examples of computer-readable mediums include, but are not limited to storage devices such as RAM, hard drives, floppy disks, CD ROMs, DVD ROMs, zip disks, tapes, magnetic storage devices, optical storage devices, MEMS-based storage devices, nanotechnology-based storage devices, and the like.

It will be recognized that the disclosed technologies are not limited to any particular computer or type of hardware. Certain details of suitable computers and hardware are well known and need not be set forth in detail in this disclosure.

### V. Conclusion

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims, and other equivalent features and acts are intended to be within the scope of the claims.

What is claimed is:

1. A device comprising:
memory; and
one or more processors coupled to the memory, the one or more processors configured to:
establish communication with a point-of-sale reader on behalf of a user, the communication initiating a purchase of at least one of one or more goods or one or more services;
authenticate the user to a store that stores aggregated healthcare data associated with the user by providing credentials of the user to the store;
obtain access to the aggregated healthcare data, which includes portions of the aggregated healthcare data that are aggregated from respective healthcare data storage systems that do not share the respective portions of the aggregated healthcare data with others of the healthcare data storage systems, via the point-of-sale reader, based at least in part on the credentials of the user and reference credentials being same; and
cause at least some of the aggregated healthcare data to be presented via a user interface that is included in the point-of-sale reader based at least in part on access to the aggregated healthcare data being obtained.

2. The device of claim 1, wherein the one or more processors include a first processor and a second processor;
wherein the device comprises:
a first chip that includes the first processor, which is configured to cause the purchase of the at least one of the one or more goods or the one or more services to be initiated by establishing the communication with the point-of-sale reader; and
a second chip that includes the second processor, which is configured to obtain the access to the aggregated healthcare data associated with the user; and
wherein the second chip is different from the first chip.

3. The device of claim 1, wherein at least one processor of the one or more processors is incorporated into a single chip; and
wherein the at least one processor is configured to establish the communication with the point-of-sale reader and to obtain the access to the aggregated healthcare data associated with the user.

4. The device of claim 1, wherein the one or more processors are configured to:
obtain the access to the aggregated healthcare data associated with the user by downloading the aggregated healthcare data through the point-of-sale reader.

5. The device of claim 1, wherein the one or more processors are further configured to:
upload information regarding the at least one of the one or more goods or the one or more services to each of the healthcare data storage systems through the point-of-sale reader.

6. The device of claim 1, wherein the one or more processors are configured to:
generate a cryptographic record of a plurality of purchases, which are initiated via a plurality of respective communications that are established with one or more point-of-sale readers and which include the purchase that is initiated by the communication with the point-of-sale reader, using blockchain technology, wherein the cryptographic record of the plurality of purchases is based on an order of the plurality of purchases.

7. The device of claim 1, wherein the one or more processors are configured to:
communicate with the point-of-sale reader by providing an account identifier that identifies a line of credit associated with the device to the point-of-sale reader and further by receiving a purchase request that requests execution of a purchase transaction corresponding to the purchase from the point-of-sale reader, the purchase request including a merchant category code of a merchant that is associated with the point-of-sale reader;
cause the merchant category code of the merchant to be cross-referenced with a plurality of healthcare-related merchant category codes; and
selectively trigger execution of the purchase transaction depending on whether the merchant category code of the merchant corresponds to at least one of the healthcare-related merchant category codes.

8. The device of claim 1, wherein the device is associated with a restricted-access credit device, which is external to the device; and
wherein the one or more processors are further configured to:
cause a portion of a cost of the at least one of the one or more goods or the one or more services that is not covered by a healthcare plan to be applied against a line of credit associated with the restricted-access credit device.

9. The device of claim 1, wherein the user is non-human animal; and
wherein the communication initiates the purchase of the at least one of the one or more goods or the one or more services for the non-human animal.

10. The device of claim 9, wherein the device is implanted under skin of the non-human animal.

11. A method of accessing healthcare data using a healthcare data chip device, the method comprising:
establishing communication with a point-of-sale reader on behalf of a user, the communication initiating a purchase of at least one of one or more goods or one or more services;
authenticating the user to a store that stores aggregated healthcare data associated with the user by providing credentials of the user to the store;
obtaining access to the aggregated healthcare data, which includes portions of the aggregated healthcare data that are aggregated from respective healthcare data storage systems that do not share the respective portions of the aggregated healthcare data with others of the healthcare data storage systems, via the point-of-sale reader, based at least in part on the credentials of the user and reference credentials being same; and
causing at least some of the aggregated healthcare data to be presented via a user interface that is included in the point-of-sale reader based at least in part on access to the aggregated healthcare data being obtained.

12. The method of claim 11, further comprising:
uploading information regarding the at least one of the one or more goods or the one or more services to each of the healthcare data storage systems through the point-of-sale reader.

13. The method of claim 11, wherein obtaining the access to the aggregated healthcare data comprises:
downloading a first portion of the aggregated healthcare data from a first healthcare data storage system to the healthcare data chip device; and
wherein the method further comprises:
storing the first portion of the aggregated healthcare data in a memory of the healthcare data chip device based at least in part on the access to the aggregated healthcare data being obtained;
uploading the first portion of the aggregated healthcare data from the memory to a second healthcare data storage system that is different from the first healthcare data storage system; and
deleting the first portion of the aggregated healthcare data from the memory based at least in part on the first portion of the aggregated healthcare data being uploaded to the second healthcare data storage system.

14. The method of claim 11, wherein the at least one of the one or more goods or the one or more services includes at least one of one or more healthcare-related goods or one or more healthcare-related services;
wherein the method further comprises:
aggregating the portions of the aggregated healthcare data from the respective healthcare data storage systems by uploading the portions to the store, including uploading information relating to the purchase of the at least one of the one or more healthcare-related goods or the one or more healthcare-related services via the point-of-sale reader to the store; and
wherein causing the at least some of the aggregated healthcare data to be presented via the user interface comprises:
causing the at least some of the aggregated healthcare data that is different from the information relating to the purchase of the at least one of the one or more healthcare-related goods or the one or more healthcare-related services to be presented via the user interface that is included in the point-of-sale reader based at least in part on the access to the aggregated healthcare data being obtained.

15. The method of claim 11, wherein the at least one of the one or more goods or the one or more services includes a medication; and
wherein the at least some of the aggregated healthcare data includes information specifying a potential consequence of mixing the medication with one or more other substances.

16. The method of claim 11, wherein the at least one of the one or more goods or the one or more services includes a first medication and a second medication; and
wherein the at least some of the aggregated healthcare data includes information specifying a potential consequence of mixing the first medication and the second medication based at least in part on the first medication and the second medication being purchased in a common transaction.

17. The method of claim 11, wherein the at least one of the one or more goods or the one or more services includes at least one of one or more healthcare-related goods or one or more healthcare-related services; and
wherein the at least some of the aggregated healthcare data includes information specifying an authorized amount that a provider is authorized to charge for the at least one of the one or more healthcare-related goods or the one or more healthcare-related services based at least in part on the purchase of the at least one of the one or more healthcare-related goods or the one or more healthcare-related services being initiated.

18. The method of claim 11, comprising:
communicating with the point-of-sale reader by providing an account identifier that identifies a line of credit associated with the healthcare data chip device to the point-of-sale reader and further by receiving a purchase request that requests execution of a purchase transaction corresponding to the purchase from the point-of-sale reader, the purchase request including a merchant category code of a merchant that is associated with the point-of-sale reader;
causing the merchant category code of the merchant to be cross-referenced with a plurality of healthcare-related merchant category codes; and
selectively triggering execution of the purchase transaction depending on whether the merchant category code of the merchant corresponds to at least one of the healthcare-related merchant category codes.

19. The method of claim 11, wherein the healthcare data chip device is associated with a restricted-access credit device that is external to the healthcare data chip device; and
wherein the method further comprises:
causing, by the healthcare data chip device, a portion of a cost of the at least one of the one or more goods or the one or more services that is not covered by a healthcare plan to be applied against a line of credit associated with the restricted-access credit device.

20. The method of claim 11, wherein the user is non-human animal;
wherein the method further comprises:
causing a bloodline of the non-human animal to be compared to a plurality of bloodlines that are identified in a bloodline database that is included in the store to identify the bloodline of the non-human animal in the bloodline database; and
causing at least one of one or more illnesses or one or more infectious diseases that occur with regard to the bloodline of the non-human animal to be identified based at least in part on the at least one of the one or more illnesses or the one or more infectious diseases being cross-referenced with the bloodline of the non-human animal in the bloodline database; and
wherein causing the at least some of the aggregated healthcare data to be presented via the user interface comprises:
causing an identity of the at least one of the one or more illnesses or the one or more infectious diseases to be presented via the user interface that is included in the point-of-sale reader.

21. A computer program product comprising a computer-readable storage medium having instructions recorded thereon for enabling a processor-based system to perform operations, the operations comprising:
establishing communication with a point-of-sale reader on behalf of a user, the communication initiating a purchase of at least one of one or more goods or one or more services;
authenticating the user to a store that stores aggregated healthcare data associated with the user by providing credentials of the user to the store;
obtaining access to the aggregated healthcare data, which includes portions of the aggregated healthcare data that are aggregated from respective healthcare data storage systems that do not share the respective portions of the aggregated healthcare data with others of the healthcare data storage systems, via the point-of-sale reader, based at least in part on the credentials of the user and reference credentials being same; and
causing at least some of the aggregated healthcare data to be presented via a user interface that is included in the point-of-sale reader based at least in part on access to the aggregated healthcare data being obtained.
